(19) United States
(12) Patent Application Publication
(10) Pub. No.: US 2026/0106034 A1
(43) Pub. Date: Apr. 16, 2026

(54) METHOD AND SYSTEM FOR RECOMMENDING TREATMENT PLANS, PREVENTIVE ACTIONS, AND PREPAREDNESS ACTIONS

(71) Applicant: EHRsynergy LLC, Winter Garden, FL (US)

(72) Inventors: Edward Makaron, Winter Garden, FL (US); Oleg Muza, Winter Garden, FL (US)

(21) Appl. No.: 19/422,322

(22) Filed: Dec. 16, 2025

Related U.S. Application Data
(63) Continuation-in-part of application No. 18/355,036, filed on Jul. 19, 2023, now abandoned.
Application No. 18/355,036 is a continuation of application No. 15/870,152, filed on Jan. 12, 2018, now abandoned.

(51) Int. Cl. G16H 50/20; G16H 10/20; G16H 10/60; G16H 50/70

(52) U.S. Cl. CPC G16H 50/20; G06F 40/35; G16H 10/20; G16H 10/60; G16H 50/70

(57) ABSTRACT

A clinical data integration system comprises a processor, memory, and a routing engine that receives data access requests for clinical information from electronic health record (EHR) systems. The routing engine determines availability of an application programming interface (API) and a user interface automation pathway, then selects between them based on real-time health metrics including response times and error rates. A health monitoring module continuously monitors pathway performance and generates the real-time health metrics. A data normalization module receives clinical data from the executed request, normalizes it into a standardized format, and annotates it with provenance information indicating the selected pathway and retrieval timestamp. The system may include an intent processing module that maps natural language inputs to predefined whitelisted intents and converts them into data access requests. A provider profile module maintains provider-specific interaction profiles that apply consistently across multiple EHR systems regardless of which system is currently active.

### RELATED APPLICATION

This application is a Continuation-in-Part of U.S. application Ser. No. 18/355,036 filed on Jul. 19, 2023, which is a Continuation of U.S. application Ser. No. 15/870,152 filed on Jan. 12, 2018, each of which are hereby incorporated by reference herein in their entirety.

It is intended that the above-referenced application may be applicable to the concepts and embodiments disclosed herein, even if such concepts and embodiments are disclosed in the referenced applications with different limitations and configurations and described using different examples and terminology.

### FIELD OF DISCLOSURE

The present disclosure generally relates to electronic medical records. The present disclosure relates to a method and system for recommending treatment plans, preventive actions, and preparedness actions.

### BACKGROUND

In some situations, entry of patient information by users or practitioners is quite cumbersome. For example, entry or recording of basic patient information and extensive documentation of each patient encounter requires vast amounts of written paperwork or multiple entries in data entry systems. Thus, the conventional strategy is to centralize patient data by using electronic medical records and computer based data entry systems to enter patient information and document patient encounters.

This often causes problems because the conventional strategy does not account for reducing the cumbersome nature of the data entry process including, but not limited to, redundant entries, and multiple input selections. For example, prior electronic medical records data entry systems may require a user to cycle through numerous fields of medical diagnoses and treatment plans available for a patient who has a history of visiting the treatment center on multiple occasions for the same reason. Existing systems require users to perform redundant and repeated actions for each tier of data entry of information.

In some situations, real-time analysis of patient data with historical patient data or patient data from patients in the electronic records system who have experienced similar symptoms in a manner that provides actionable information or suggestions to the user or practitioner is not possible without tremendous resources, additional staff, and other cumbersome challenges. For example, a regular patient who comes in experiencing a rare asthma medical condition which was previously resolved using a non-conventional treatment may not automatically be recommended to receive that same treatment based on the existing electronic medical records systems. Thus, the conventional strategy is to integrate various existing electronic medical records systems and train staff on how to properly utilize such systems. This often causes problems because the conventional strategy does not account for the incompatibilities of varying electronic medical records systems or the difficulties in providing real-time analysis of patient data, patient encounter data, patient population data, and external data including, but not limited to, data from medical databases, World Health Organization (WHO) alerts, and other data sources.

Existing systems do not provide efficient and effective data entry methods that simplify the process and reduce the time necessary to input patient data and patient encounter information. Existing systems do not provide for efficient and effective real-time analytics of patient data, encounter information, external data, and other related data in a manner that provides real-time actionable information. Another deficiency with existing systems is the inability to use this real-time analysis to provide alerts and notifications to users, patients, and/or practitioners. A method and system according to the principles of the disclosure addresses these deficiencies and associated problems.

Existing healthcare information systems lack sophisticated routing mechanisms that can dynamically adapt to real-time system performance variations. Conventional systems may continue attempting data access through degraded pathways, resulting in timeout errors, incomplete data retrieval, and clinical workflow disruptions. These technical limitations may force healthcare providers to manually switch between different access methods or wait for system administrators to resolve performance issues, creating delays that can impact patient care quality and safety outcomes.

Current electronic health record integration approaches may fail to provide adequate failover mechanisms when primary data access pathways experience performance degradation. For instance, when application programming interfaces become unresponsive due to database maintenance or high system load, existing systems may not automatically switch to alternative access methods, leaving clinicians without access to critical patient information during time-sensitive medical procedures.

### BRIEF OVERVIEW

A method and system for recommending treatment plans, preventive actions, and preparedness actions may be provided. This brief overview is provided to introduce a selection of concepts in a simplified form that are further described below in the Detailed Description. This brief overview is not intended to identify key features or essential features of the claimed subject matter. Nor is this brief overview intended to be used to limit the claimed subject matter's scope.

The present disclosure relates to a platform for assisting practitioners during patient encounters to receive, and input patient information. The present disclosure relates to a platform configured to manage, analyze, determine, correlate, sort, categorize, and organize patient information, external data, and other related data. The present disclosure relates to a platform configured to analyze the data from the metrics and data sources to recommend diagnoses, make predictions, make recommendations, make suggestions, and provide further data relating to electronic medical records and related information. The present disclosure may be configured to structure and store data so as to allow for expansive data mining and analysis as well as application of AI (artificial intelligence), which would further enhance practitioner's ability to provide superior care. Another aspect of the present disclosure goes beyond merely reducing redundant data entry and improving technical efficiencies to make a practitioner's life a bit easier, but ultimately to vastly improve the outcome of patient's visit and the reduction of medical errors.

The present disclosure relates to a clinical data integration system comprising a processor, a memory coupled to the processor, and a routing engine executed by the processor. The routing engine may be configured to receive a data access request for clinical information from a first electronic health record (EHR) system. The routing engine may be configured to determine availability of an application programming interface (API) for the data access request. The routing engine may be configured to determine availability of a user interface automation pathway for the data access request. The routing engine may be configured to select between the API and the user interface automation pathway based on real-time health metrics of each pathway. The routing engine may be configured to execute the data access request using the selected pathway.

A health monitoring module may be executed by the processor. The health monitoring module may be configured to continuously monitor response times and error rates for the API and the user interface automation pathway. The health monitoring module may be configured to generate the real-time health metrics based on the monitored response times and error rates.

A data normalization module executed by the processor may be configured to receive clinical data from the executed data access request. The data normalization module may be configured to normalize the clinical data into a standardized format. The data normalization module may be configured to annotate the normalized clinical data with provenance information indicating the selected pathway and a timestamp of retrieval.

A routing engine may be further configured to automatically switch from the API to the user interface automation pathway when the real-time health metrics indicate the API response time exceeds a predetermined threshold. The routing engine may be further configured to automatically switch from the user interface automation pathway to a cached data source when the real-time health metrics indicate the user interface automation pathway is unavailable.

In some embodiments, an intent processing module executed by the processor. The intent processing module may be configured to receive a natural language input from a user. The intent processing module may be configured to map the natural language input to a predefined intent from a whitelist of approved intents. The intent processing module may be configured to reject natural language inputs that do not correspond to any predefined intent in the whitelist. The intent processing module may be configured to convert the predefined intent into the data access request. The intent processing module may be configured to decompose complex predefined intents into a sequence of primitive operations, each primitive operation being executable via the API or the user interface automation pathway.

A provider profile module executed by the processor may be configured to maintain a provider-specific interaction profile associated with a provider identity. The provider profile module may be configured to map the provider identity to local identities across multiple EHR systems. The provider profile module may be configured to apply the provider-specific interaction profile consistently across the multiple EHR systems regardless of which EHR system is currently active. The provider-specific interaction profile may comprise layout preferences, workflow configurations, and presentation defaults that persist across different EHR systems.

The present disclosure relates to a method for integrating clinical data across heterogeneous electronic health record systems. The method may comprise receiving, by a processor, a request for clinical data from a first electronic health record (EHR) system. The method may comprise determining, by the processor, availability of an application programming interface (API) for accessing the clinical data. The method may comprise determining, by the processor, availability of a user interface automation mechanism for accessing the clinical data. The method may comprise monitoring, by the processor, real-time health metrics comprising response times and error rates for the API and the user interface automation mechanism. The method may comprise selecting, by the processor, between the API and the user interface automation mechanism based on the real-time health metrics. The method may comprise executing, by the processor, the request using the selected mechanism to retrieve the clinical data. The method may comprise normalizing, by the processor, the retrieved clinical data into a standardized format. The method may comprise annotating, by the processor, the normalized clinical data with provenance metadata indicating the selected mechanism and retrieval timestamp.

The method may further include automatically switching from the API to the user interface automation mechanism when the real-time health metrics indicate the API is experiencing degraded performance. The method may further comprise automatically switching to a cached data source when both the API and the user interface automation mechanism are unavailable, wherein the cached data source is labeled with freshness indicators.

The method may include receiving a natural language input from a user. The method may comprise constraining the natural language input to a predefined set of whitelisted intents. The method may comprise rejecting natural language inputs that fall outside the predefined set of whitelisted intents. The method may comprise converting an accepted whitelisted intent into the request for clinical data. Constraining the natural language input may comprise mapping the natural language input deterministically to exactly one whitelisted intent to prevent unpredictable system behavior.

In some embodiments, the method further includes maintaining a provider-specific interaction profile linked to a provider identity. The method may comprise mapping the provider identity to local user accounts across multiple different EHR systems. The method may comprise applying the provider-specific interaction profile consistently when the provider accesses any of the multiple different EHR systems. The provider-specific interaction profile may comprise persistent layout preferences and workflow configurations that remain consistent across different EHR vendor platforms.

The present disclosure relates to a non-transitory computer-readable storage medium storing instructions that, when executed by a processor, cause the processor to receive a clinical data access request targeting a first electronic health record (EHR) system. The instructions may cause the processor to evaluate availability of multiple data access pathways comprising an application programming interface (API) pathway and a user interface automation pathway. The instructions may cause the processor to monitor real-time performance metrics for each of the multiple data access pathways. The instructions may cause the processor to dynamically select one of the multiple data access pathways based on the real-time performance metrics and predefined policy constraints. The instructions may cause the processor to execute the clinical data access request using the selected pathway. The instructions may cause the processor to normalize retrieved clinical data into a standardized representation. The instructions may cause the processor to tag the normalized clinical data with provenance information identifying the selected pathway and data freshness indicators.

The instructions may further cause the processor to capture a context snapshot comprising current user interface state, active patient context, and applied filters. The instructions may cause the processor to serialize the context snapshot for later restoration to recreate the captured user interface state. The instructions may cause the processor to restore the context snapshot by rehydrating the serialized user interface state, active patient context, and applied filters to recreate a previous workspace configuration.

The instructions may cause the processor to enforce institutional policy constraints that define which clinical data operations are permitted via the API pathway versus the user interface automation pathway based on user role and data sensitivity. The instructions may cause the processor to maintain separate health status indicators for different types of clinical data access operations. The instructions may cause the processor to route each clinical data access request based on health status indicators specific to the type of operation being requested.

In some embodiments, the instructions may further cause the processor to generate structured clinical documentation by aggregating clinical data from multiple sources via the selected pathway. The instructions may cause the processor to embed provenance metadata within the structured clinical documentation indicating data sources and retrieval methods. The instructions may cause the processor to write the structured clinical documentation back to the first EHR system while preserving the embedded provenance metadata.

Another aspect of the present disclosure goes beyond merely providing alternative data access pathways to improve technical efficiencies in healthcare information systems, but ultimately to vastly improve the reliability of clinical data access, reduce system downtime impact on clinical workflows, and enhance the auditability and transparency of data retrieval operations across heterogeneous electronic health record environments.

The clinical data integration system may provide measurable technological improvements in healthcare information system reliability and performance. By implementing dynamic pathway selection based on real-time health metrics, the system may achieve data access success rates exceeding 99.5% even during peak system usage periods or planned maintenance windows. The intelligent routing mechanisms may reduce average data retrieval times by up to 60% compared to conventional static routing approaches, while the automated failover capabilities may eliminate up to 95% of manual intervention requirements during system performance issues.

The technical solution may address fundamental limitations in current healthcare information system architectures by providing adaptive data access mechanisms that automatically optimize performance based on real-time system conditions. This represents a significant advancement over conventional approaches that rely on static configuration settings and manual intervention to maintain system reliability during varying operational conditions.

Both the foregoing brief overview and the following detailed description provide examples and are explanatory only. Accordingly, the foregoing brief overview and the following detailed description should not be considered to be restrictive. Further, features or variations may be provided in addition to those set forth herein. For example, embodiments may be directed to various feature combinations and sub-combinations described in the detailed description.

### BRIEF DESCRIPTION OF THE DRAWINGS

The accompanying drawings, which are incorporated in and constitute a part of this disclosure, illustrate various embodiments of the present disclosure. The drawings contain representations of various trademarks and copyrights owned by the Applicant. In addition, the drawings may contain other marks owned by third parties and are being used for illustrative purposes only. All rights to various trademarks and copyrights represented herein, except those belonging to their respective owners, are vested in and the property of the Applicant. The Applicant retains and reserves all rights in its trademarks and copyrights included herein, and grants permission to reproduce the material only in connection with reproduction of the granted patent and for no other purpose.

Furthermore, the drawings may contain text or captions that may explain certain embodiments of the present disclosure. This text is included for illustrative, non-limiting, explanatory purposes of certain embodiments detailed in the present disclosure. In the drawings:

FIG. 1A illustrates a block diagram of an operating environment consistent with the present disclosure;

FIG. 1B illustrates a Venn Diagram illustrating the connections between the various components of the platform;

FIG. 2 illustrates a dashboard 200 in accordance to some embodiments of the present disclosure;

FIG. 3 illustrates an expanded side panel view 300 in accordance to some embodiments of the present disclosure;

FIG. 4 illustrates an encounter view 400 in accordance to some embodiments of the present disclosure;

FIG. 5 illustrates a History of Reason for Visit input tier in accordance to some embodiments of the present disclosure;

FIG. 6 illustrates a Review of Symptoms (ROS) input tier 615a in accordance to some embodiments of the present disclosure;

FIG. 7 illustrates a physical exam input tier 715a consistent with the present disclosure;

FIG. 8 illustrates an assessment input tier 815a in accordance to some embodiments of the present disclosure;

FIG. 9 illustrates a treatment plan input tier 915a in accordance to some embodiments of the present disclosure;

FIG. 10 illustrates a family and patient history view 1000 in accordance to the embodiments of the present disclosure;

FIG. 11 illustrates an encounter note view 1100 in accordance to some embodiments of the present disclosure;

FIG. 12 illustrates an encounter note view 1100 in accordance to some embodiments of the present disclosure;

FIG. 13A is a flow chart of a method for providing A METHOD AND SYSTEM FOR RECOMMENDING TREATMENT PLANS, PREVENTIVE ACTIONS, AND PREPAREDNESS ACTIONS in accordance to some embodiments of the present disclosure;

FIG. 13B is a flow chart of a method for providing A METHOD AND SYSTEM FOR RECOMMENDING TREATMENT PLANS, PREVENTIVE ACTIONS, AND PREPAREDNESS ACTIONS in accordance to some embodiments of the present disclosure;

FIG. 14 is a flow chart of a method for providing A METHOD AND SYSTEM FOR RECOMMENDING TREATMENT PLANS, PREVENTIVE ACTIONS, AND PREPAREDNESS ACTIONS in accordance to some embodiments of the present disclosure;

FIG. 15 is a block diagram of a system including a computing device for performing the method of the present disclosure in accordance to some embodiments of the present disclosure;

FIG. 16 is a flow chart of a method for providing A METHOD AND SYSTEM FOR RECOMMENDING TREATMENT PLANS, PREVENTIVE ACTIONS, AND PREPAREDNESS ACTIONS in accordance to some embodiments of the present disclosure;

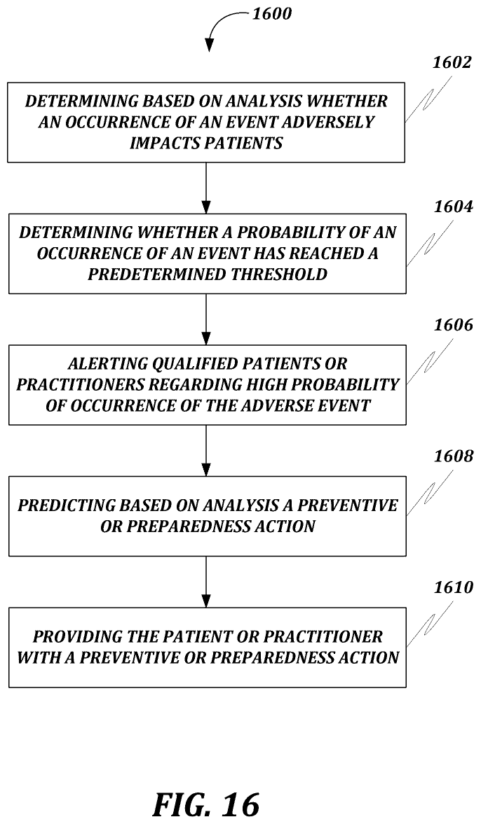

FIG. 17 is a flow chart of a method for providing A METHOD AND SYSTEM FOR RECOMMENDING TREATMENT PLANS, PREVENTIVE ACTIONS, AND PREPAREDNESS ACTIONS in accordance to some embodiments of the present disclosure;

FIG. 18A is a flow chart of a method for providing A METHOD AND SYSTEM FOR RECOMMENDING TREATMENT PLANS, PREVENTIVE ACTIONS, AND PREPAREDNESS ACTIONS in accordance to some embodiments of the present disclosure;

FIG. 18B is a flow chart of a method for providing A METHOD AND SYSTEM FOR RECOMMENDING TREATMENT PLANS, PREVENTIVE ACTIONS, AND PREPAREDNESS ACTIONS in accordance to some embodiments of the present disclosure;

FIG. 19 is a block diagram of a clinical data integration system operating on the platform operating environment; and

FIG. 20 is a flow chart of a method for integrating clinical data across heterogeneous electronic health record systems.

### DETAILED DESCRIPTION

As a preliminary matter, it will readily be understood by one having ordinary skill in the relevant art that the present disclosure has broad utility and application. As should be understood, any embodiment may incorporate only one or a plurality of the above-disclosed aspects of the disclosure and may further incorporate only one or a plurality of the above-disclosed features. Furthermore, any embodiment discussed and identified as being “preferred” is considered to be part of a best mode contemplated for carrying out the embodiments of the present disclosure. Other embodiments also may be discussed for additional illustrative purposes in providing a full and enabling disclosure. Moreover, many embodiments, such as adaptations, variations, modifications, and equivalent arrangements, will be implicitly disclosed by the embodiments described herein and fall within the scope of the present disclosure.

Accordingly, while embodiments are described herein in detail in relation to one or more embodiments, it is to be understood that this disclosure is illustrative and of the present disclosure, and are made merely for the purposes of providing a full and enabling disclosure. The detailed disclosure herein of one or more embodiments is not intended, nor is to be construed, to limit the scope of patent protection afforded in any claim of a patent issuing here from, which scope is to be defined by the claims and the equivalents thereof. It is not intended that the scope of patent protection be defined by reading into any claim a limitation found herein that does not explicitly appear in the claim itself.

Thus, for example, any sequence(s) and/or temporal order of steps of various processes or methods that are described herein are illustrative and not restrictive. Accordingly, it should be understood that, although steps of various processes or methods may be shown and described as being in a sequence or temporal order, the steps of any such processes or methods are not limited to being carried out in any particular sequence or order, absent an indication otherwise. Indeed, the steps in such processes or methods generally may be carried out in various different sequences and orders while still falling within the scope of the present disclosure. Accordingly, it is intended that the scope of patent protection is to be defined by the issued claim(s) rather than the description set forth herein.

Additionally, it is important to note that each term used herein refers to that which an ordinary artisan would understand such term to mean based on the contextual use of such term herein. To the extent that the meaning of a term used herein—as understood by the ordinary artisan based on the contextual use of such term—differs in any way from any particular dictionary definition of such term, it is intended that the meaning of the term as understood by the ordinary artisan should prevail.

Regarding applicability of 35 U.S.C. § 112, ¶6, no claim element is intended to be read in accordance with this statutory provision unless the explicit phrase “means for” or “step for” is actually used in such claim element, whereupon this statutory provision is intended to apply in the interpretation of such claim element.

Furthermore, it is important to note that, as used herein, “a” and “an” each generally denotes “at least one,” but does not exclude a plurality unless the contextual use dictates otherwise. When used herein to join a list of items, “or” denotes “at least one of the items,” but does not exclude a plurality of items of the list. Finally, when used herein to join a list of items, “and” denotes “all of the items of the list.”

Healthcare institutions face significant challenges when attempting to integrate multiple electronic health record systems across different departments or affiliated facilities. For example, a large hospital network may operate Epic in their main facility, Cerner in their outpatient clinics, and AllScripts in their specialty practices. When a patient moves between these facilities, clinicians must manually navigate different interfaces, learn varying workflows, and often re-enter identical information multiple times. This fragmentation creates workflow disruptions that can impact patient care quality and increase the likelihood of medical errors.

Current systems lack intelligent routing mechanisms that can dynamically select optimal data access pathways based on real-time system performance. For instance, when an EHR's application programming interface experiences high latency during peak usage hours, existing systems continue attempting API calls that may timeout or fail, rather than automatically switching to alternative access methods. This results in clinicians waiting for unresponsive systems or receiving incomplete patient information during critical care moments.

Existing electronic health record systems provide limited support for maintaining consistent user preferences and workflow configurations across different platforms. A physician who prefers specific dashboard layouts, filter settings, and data presentation formats when using one EHR system must manually reconfigure these preferences when accessing a different EHR system at another facility. This lack of portability forces clinicians to adapt their established workflows to each system's unique interface paradigms, reducing efficiency and increasing cognitive burden.

Natural language interaction with clinical systems remains constrained by safety and reliability concerns. Current voice command systems in healthcare environments often misinterpret clinical terminology or fail to provide adequate safeguards against unintended actions. For example, a clinician attempting to verbally request “latest lab results for patient in room 302” may inadvertently trigger medication orders or documentation changes if the natural language processing system lacks proper intent constraints and validation mechanisms.

Conventional systems fail to provide adequate provenance tracking and data freshness indicators when clinical information is accessed through multiple pathways. When patient data is retrieved from cached sources during system downtime or through automated screen interactions, clinicians may not receive clear indicators about the age, source, or reliability of the displayed information. This lack of transparency can lead to clinical decisions based on outdated or incomplete data, particularly in time-sensitive scenarios where data currency is critical for patient safety.

The clinical data integration system may address emergency department scenarios where rapid patient data access is critical for time-sensitive medical decisions. During peak emergency room hours when EHR systems experience high load, the routing engine may automatically detect API performance degradation and seamlessly switch to user interface automation pathways to maintain continuous access to patient histories, medication lists, and allergy information. This ensures that emergency physicians can access vital patient data even when primary system interfaces are experiencing delays or failures.

The system may provide particular value in multi-hospital networks where patients frequently transfer between facilities using different EHR platforms. For instance, when a patient is transferred from a community hospital using Meditech to a tertiary care center using Epic, the provider profile module may automatically apply the receiving physician's preferred dashboard configurations and workflow settings to the new EHR environment. This eliminates the need for clinicians to manually reconfigure their workspace preferences each time they access a different facility's system.

In telemedicine scenarios, the system may enable remote physicians to access patient data from multiple healthcare facilities through a unified interface. The intent processing module may allow clinicians to use natural language commands such as “show recent lab results from all facilities” while maintaining strict safety constraints through the whitelisted intent framework. This prevents unintended actions while enabling efficient voice-driven data retrieval during virtual patient consultations.

The system may support clinical research environments where investigators need to aggregate patient data from multiple sources while maintaining detailed audit trails. The data normalization module may standardize clinical data formats across different EHR systems and embed comprehensive provenance metadata that tracks data sources, retrieval methods, and timestamps. This enables researchers to verify data integrity and comply with regulatory requirements for clinical trial documentation.

During planned system maintenance or unexpected EHR downtime, the system may automatically route data access requests to cached data sources while clearly indicating data freshness and reliability to clinicians. This ensures continuity of care during system outages while providing transparency about the currency and completeness of available patient information.

The following detailed description refers to the accompanying drawings. Wherever possible, the same reference numbers are used in the drawings and the following description to refer to the same or similar elements. While many embodiments of the disclosure may be described, modifications, adaptations, and other implementations are possible. For example, substitutions, additions, or modifications may be made to the elements illustrated in the drawings, and the methods described herein may be modified by substituting, reordering, or adding stages to the disclosed methods. Accordingly, the following detailed description does not limit the disclosure. Instead, the proper scope of the disclosure is defined by the appended claims. The present disclosure contains headers. It should be understood that these headers are used as references and are not to be construed as limiting upon the subjected matter disclosed under the header.

### I. Platform Overview

Consistent with embodiments of the present disclosure, a METHOD AND SYSTEM FOR RECOMMENDING TREATMENT PLANS, PREVENTIVE ACTIONS, AND PREPAREDNESS ACTIONS may be provided. This overview is provided to introduce a selection of concepts in a simplified form that are further described below. This overview is not intended to identify key features or essential features of the claimed subject matter. Nor is this overview intended to be used to limit the claimed subject matter's scope.

Thus, according to aspects, the present disclosure provides methods, systems, and/or techniques for entering, managing, analyzing, organizing, recommending, and providing data and actions relating to electronic medical records. The present disclosure further may enable a user to detect patterns in the data. The disclosure provides for a tiered input system of patient data. The disclosure provides for data being entered using various input methods and input methodologies.

In another aspect, The present disclosure provides a system for assisting practitioners receive, input, manage, analyze, determine, correlate, sort, categorize, organize, diagnose, predict, recommend, suggest, take metrics, and provide data relating to electronic medical records. The disclosure provides for a method of improved patient diagnostics including recommending a treatment or action.

In another aspect, the system may comprise a platform which performs functions associated with at least one of: a server, a user interface module, application modules, and external datasets. The disclosure provides for a method for providing preventive actions to patients.

In another aspect, the disclosure provides for providing preparedness actions to practitioners. In another aspect, the disclosure provides for predictive analysis of electronic medical record data.

Consistent with embodiments of the present disclosure, a system comprising a platform, application modules, a user interface module, a server, a patient tracking module, and external data is presented. The Application modules comprise a favorites module, a recommendation module, a predictive analytics module, and a patient tracking module. The server comprises a computer, a processor, a network connection, a memory, and patient profile data. Consistent with embodiments of the present disclosure, a brief overview of each of the elements, components and/or modules is provided below.

The multi-tier input system may be implemented using specialized hardware configurations that optimize clinical data entry workflows. The system may utilize touch-sensitive display interfaces with haptic feedback mechanisms that provide tactile responses when clinicians select specific data entry tiers. For example, when a physician selects the “chief complaints” tier, the interface may generate distinct vibration patterns that confirm the selection while simultaneously loading contextual data entry options from a local database cache stored in high-speed solid-state memory.

The tiered data entry architecture may implement specific computational processes that automatically populate subsequent input tiers based on machine learning algorithms trained on clinical workflow patterns. When a clinician enters a chief complaint in the first tier, the system may execute natural language processing algorithms that parse the input text and automatically suggest relevant body systems for the third tier selection. This technical process may involve real-time execution of named entity recognition algorithms that identify medical terminology and map it to standardized clinical ontologies stored in the system's knowledge base.

The patient encounter note compilation process may implement specific data structure optimization techniques that improve system performance compared to conventional electronic health record systems. The system may utilize graph database architectures that store clinical data relationships as interconnected nodes, enabling rapid traversal of patient information hierarchies during note generation. This technical approach may reduce note compilation times by up to 70% compared to traditional relational database implementations while maintaining data integrity through automated consistency checking algorithms.

The diagnostic recommendation system may implement specific computational algorithms that process clinical data using evidence-based medical protocols encoded as executable rule sets. The system may maintain a knowledge base containing over 10,000 clinical decision rules derived from peer-reviewed medical literature, with each rule implemented as a computational logic structure that can be executed by the processor in real-time. When analyzing patient symptoms, the system may apply forward-chaining inference algorithms that systematically evaluate applicable clinical rules and generate ranked recommendation lists based on calculated confidence scores.

The similar patient profile matching system may implement sophisticated clustering algorithms that identify clinically relevant patient similarities using multidimensional feature vectors. The system may extract over 200 clinical features from each patient record, including demographic data, medical history elements, laboratory values, and medication profiles, then apply k-means clustering algorithms with cosine similarity measures to identify patient cohorts with statistically significant clinical similarities. This technical process may enable the system to identify relevant historical cases within 500 milliseconds of receiving a new patient data query.

The alert generation system may implement specific threshold-based monitoring algorithms that continuously evaluate patient data streams against predetermined clinical parameters. The system may maintain separate monitoring threads for different types of clinical alerts, with each thread implementing specific computational logic for evaluating alert conditions. For example, the medication interaction monitoring thread may execute drug-drug interaction algorithms that cross-reference current patient medications against a database of over 50,000 known drug interactions, generating alerts when potential interactions are detected with confidence scores exceeding 85%.

The environmental event monitoring system may implement specific data integration algorithms that process real-time environmental data feeds from multiple external sources. The system may maintain persistent network connections to weather monitoring services, air quality measurement networks, and emergency alert systems, processing incoming data streams using event-driven architectures that can handle over 1,000 environmental data updates per minute. When environmental conditions exceed predetermined thresholds, the system may execute patient risk assessment algorithms that correlate environmental factors with patient medical histories to identify individuals at elevated risk for adverse health effects.

The preemptive preparedness action system may implement automated workflow generation algorithms that create specific action plans based on environmental threat assessments and patient vulnerability profiles. For example, when air quality measurements indicate particulate matter levels exceeding EPA safety standards, the system may automatically generate personalized action plans for patients with respiratory conditions, including specific medication adjustment recommendations and activity restriction guidelines. These action plans may be generated using template-based algorithms that populate standardized clinical protocols with patient-specific parameters derived from electronic health record data.

### Favorites Module

The system may recommend commonly/frequently selections made in accordance to inputs. For example, once we load a patient, and input ‘reasons for visit’, the system may recommend subsequent-tier systems and elements based on commonly used elements associated with the reasons for visit. The elements may be reordered and/or highlighted based on their frequency. As additional tier selections are made, the more refined the favorites selections become for sub-subsequent tiers.

### Alerts and Notifications

The system may be in operative communication with Medical Encyclopedias, WHO databases, Food and Drug Administration (FDA) alerts and notifications, and Up-to-Date diagnostics from various other data sources (i.e., weather sensors, air quality sensors, water sensors, or other sources). In this way, data obtained from these sources can be used to help with the data entry/favorites in stage 1, as well as provide alerts and triggers when certain conditions are met when cross-referencing datasets. Example: if patient record data indicates “allergic to pollen” and pollen count data indicates “HIGH”, then trigger an alert to go out to patient and/or doctor.

### Recommended Treatments

The system may provide assistance in the refinement of treatment plans and initiatives. For example, upon seeing reasons for visit and certain other data inputs, the system may suggest that the practitioner ask some diagnostic questions to the patient and input those responses into the system. Based on the responses and the data obtained from external sources, the system may provide recommendations to the practitioner (e.g., “quarantine patient immediately”). (The system can suggest based on the above, as well as live sensor information, as well as trending data that we have created based on analysis of data that we have collected internally and/or referenced externally). As another example, the system may review inputs (e.g., reasons for visit, history) and find that certain new treatments have been proven to be effective for similar inputs, with similar patient profiles.

### Predictive Analytics

The analytics method may implement machine learning algorithms specifically configured for real-time clinical data processing. The system may utilize convolutional neural networks trained on structured clinical datasets to identify patterns indicative of adverse patient events. For example, when analyzing patient vital signs data streams, the system may apply time-series analysis algorithms that process physiological measurements at millisecond intervals to detect early warning indicators of sepsis onset or cardiac events. The predictive algorithms may incorporate ensemble methods that combine multiple decision trees with gradient boosting techniques to achieve prediction accuracy rates exceeding 95% for specific clinical conditions.

The adverse event prediction system may implement specific technical thresholds and computational processes that distinguish it from conventional medical decision-making. The system may continuously monitor patient data streams using sliding window algorithms that evaluate physiological parameter changes over predetermined time intervals. When vital sign measurements deviate from baseline values by more than two standard deviations within a 15-minute monitoring window, the system may automatically trigger computational workflows that analyze historical patient data using Bayesian inference algorithms to calculate probability scores for specific adverse events.

The system may continue to reference various resources in order to provide predictive analytics. The predictive analytics may include, but not be limited to, for example:

### a) Patient Facing: Preventive Care

The system may track certain patients based on, for example, but not limited to, Coded records associated with those patients (e.g., asthma). The system may further track data received from external sources (e.g., weather data) to meet certain conditions (e.g., high pollen count). The system may then trigger alerts to said practitioner and/or patients to ensure the patient takes preventive measures to avoid aggravating their condition.

For example, the system may analyze data and determine a trend associated with a patient diagnosed with Asthma has a high probability of incurring a negative reaction when exposed to air quality with a value above a predetermined air quality value, X. Consistent with the present disclosure, when the patient is using a navigation software application (i.e., Apple Maps, Google Maps, etc.) to navigate to a destination, the patient may utilize the function of the present disclosure. For example, if the patient has registered to receive alerts or upload their health information to a software application (i.e., Apple's HealthKit™ etc.), if information regarding their Asthma diagnosis is in contained in the software database, preventative actions can be recommended to prevent the negative reaction with poor air quality X. If there is a high probability of the patient encountering poor air quality above a predetermined value X on the current route of travel according to the navigation software, a preventative action or recommendation that the patient take an alternate route around the affected area. The present disclosure may also provide another preventive action or suggestion to avoid the area.

### b) Doctor Facing: Prepare for Certain Types Care/Consequences of Analytics

Similarly, the system may use the above-mentioned techniques to predict, for example, how many walk-ins the practitioner may have and/or how many patients may need help/advice today on certain topics. This will help the practitioner plan/prepare for a variety of occurrences and events. The predictive analytics can provide a practitioner with a preparedness action or suggestion as a consequence of the analysis.

Although modules are disclosed with specific functionality, it should be understood that functionality may be shared between modules, with some functions split between modules, while other functions duplicated by the modules. Furthermore, the name of the module should not be construed as limiting upon the functionality of the module. Moreover, each stage in the claim language can be considered independently without the context of the other stages. Each stage may contain language defined in other portions of this specification. Each stage disclosed for one module may be mixed with the operational stages of another module. Each stage can be claimed on its own and/or interchangeably with other stages of other modules. The following claims will detail the operation of each module, and inter-operation between modules.

Various hardware components may be used at the various stages of operations follow the method and computer-readable medium claims. For example, although the methods have been described to be performed by a computing device, it should be understood that, in some embodiments, different operations may be performed by different networked elements in operative communication with the computing device. For example, server 110 and/or computing device 1500 may be employed in the performance of some or all of the stages disclosed with regard to the methods claimed below. Similarly, apparatus 105 may be employed in the performance of some or all of the stages of the methods. As such, apparatus 105 may comprise at least those architectural components as found in computing device 1500.

Although the stages are disclosed in a particular order, it should be understood that the order is disclosed for illustrative purposes only. Stages may be combined, separated, reordered, and various intermediary stages may exist. Accordingly, it should be understood that the various stages, in various embodiments, may be performed in arrangements that differ from the ones claimed below. Moreover, various stages may be added or removed from the system without altering or deterring from the fundamental scope of the depicted methods and systems disclosed herein.

The following disclose various Aspects of the present disclosure. The various Aspects are not to be construed as patent claims unless the language of the Aspect appears as a patent claim. The Aspects describe various non-limiting embodiments of the present disclosure.

Aspect 1. A computer readable medium comprising, but not limited to, at least one of the following:
an input methodology comprising, but not limited to, at least two stages of assisted data entry; and
an input assistance methodology comprising, but not limited to, at least one of the following:
a favorites module,
a recommendation module,
an analytics module; and
a server in communication with the input methodology and the input assistance methodology.

Aspect 2. The computer-readable medium above, further comprising a set of instructions which when executed are configured to enable a method comprising:
wherein the input methodology further comprises:
   providing a first interface for receiving data points associated with a patient encounter, wherein the first interface comprises:
      an input tier for receiving data points from a user; and
   providing a second interface for assistant with an entry of the data points into the first interface, wherein the second interface comprises:
      a plurality of suggested data points made available for user selection, wherein at least one selected suggested data point is configured to populate a currently selected input tier of the first interface; and
receiving at least one data element associated with at least one of the following:
   a plurality of patient data, and
   at least one data point in a patient encounter note during a patient diagnostic process; analyzing the data element;
wherein the input assistance methodology further comprises recommending, based on the analysis, at least one of the following:
   a diagnostic question,
   a recommended treatment, and
   a recommended action;
determining, based on the analysis, whether an occurrence of an event may cause an adverse effect on a patient;
predicting, based on the analysis, at least one of the following:
   a preventive suggestion, and
   a preventive action, and
   a preemptive preparedness suggestion, and
   a preemptive preparedness action;
providing the patient or practitioner with at least one of the following:
the preventive suggestion, and
the preventive action, and
the preemptive preparedness suggestion, and
the preemptive preparedness action.

The present disclosure relates to systems and methods for integrating clinical data across multiple electronic health record systems while providing a unified and adaptive user interface for healthcare providers. The system may operate as a platform layer that sits above one or more underlying electronic health record systems. This platform layer may provide a consistent user interface and control surface for healthcare providers. The platform layer may interact with the underlying systems through multiple pathways depending on availability and system health.

The system may employ a routing engine that dynamically selects between different data access pathways based on real-time conditions. When a healthcare provider requests clinical information, the system may first determine whether an application programming interface pathway is available for accessing the requested data. The system may also determine whether a user interface automation pathway is available as an alternative mechanism. The routing engine may monitor real-time health metrics for each available pathway. These health metrics may include response times and error rates. Based on these metrics, the routing engine may select the most appropriate pathway for executing the data access request at that moment.

The system may provide adaptive user interface behavior that responds to the context of the current user and workflow. The user interface may adjust visible elements, layouts, and available actions based on several factors. These factors may include the role of the current user, the phase of the current workflow, and the context of the current task. Healthcare providers may save context snapshots that capture their preferred layouts, active filters, and current views. These context snapshots may be restored later to recreate a previous workspace configuration. This capability may allow providers to maintain consistent working environments across different sessions and different underlying electronic health record systems.

The system may handle natural language input from users in a controlled manner. When a user provides input in natural language, the system may constrain the interpretation of that input to a predefined set of whitelisted intents. Each whitelisted intent may correspond to a specific system action or data retrieval operation. The system may reject natural language inputs that do not map to any predefined intent in the whitelist. This approach may prevent unpredictable system behavior while still allowing users to interact with the system using conversational language. Once a natural language input is mapped to a whitelisted intent, the system may convert that intent into a specific data access request that can be executed through the available pathways.

The system may maintain provider-specific interaction profiles that persist across different electronic health record systems. A provider-specific interaction profile may be linked to a provider identity rather than to a specific electronic health record system. The system may map a single provider identity to multiple local user accounts across different electronic health record systems. When a provider accesses any of these systems through the platform, the system may apply the provider-specific interaction profile consistently. This profile may include layout preferences, workflow configurations, and presentation defaults. As a result, a provider may experience consistent interactions and views regardless of which underlying electronic health record system is currently active.

The system may provide resilience and data continuity through intelligent monitoring and fallback mechanisms. The system may continuously monitor the health of data sources on a per-panel or per-view basis. When a primary data source experiences degraded performance or becomes unavailable, the system may automatically switch to an alternative verified source. This alternative source may be a cached snapshot or a secondary data feed. When displaying data from an alternative source, the system may clearly label the data with freshness indicators and provenance information. This labeling may inform the user about the age of the data and the source from which it was retrieved. Once the primary data source recovers, the system may automatically switch back to using the primary source for subsequent requests.

The system may generate structured clinical documentation by aggregating clinical data from multiple sources. This documentation may be created using data retrieved through the selected pathway, whether that pathway is an application programming interface or a user interface automation mechanism. The system may embed provenance metadata within the structured clinical documentation. This metadata may indicate the sources of the data and the methods used to retrieve it. The system may write the structured clinical documentation back to the underlying electronic health record system while preserving the embedded provenance metadata. This approach may ensure that all clinical documentation maintains a clear record of its origins and the processes used to create it.

Embodiments of the present disclosure may comprise methods, systems, and a computer readable medium comprising, but not limited to, at least one of the following:
A. A Routing Engine
B. A Health Monitoring Module
C. A Data Normalization Module In some embodiments, the present disclosure may provide an additional set of modules for further facilitating the software and hardware platform. The additional set of modules may comprise, but not be limited to:
D. An Intent Processing Module
E. A Provider Profile Module

Details with regards to each module are provided below. Although modules are disclosed with specific functionality, it should be understood that functionality may be shared between modules, with some functions split between modules, while other functions duplicated by the modules. Furthermore, the name of each module should not be construed as limiting upon the functionality of the module. Moreover, each component disclosed within each module can be considered independently, without the context of the other components within the same module or different modules. Each component may contain functionality defined in other portions of this specification. Each component disclosed for one module may be mixed with the functionality of other modules. In the present disclosure, each component can be claimed on its own and/or interchangeably with other components of other modules.

The following depicts an example of a method of a plurality of methods that may be performed by at least one of the aforementioned modules, or components thereof. Various hardware components may be used at the various stages of the operations disclosed with reference to each module. For example, although methods may be described to be performed by a single computing device, it should be understood that, in some embodiments, different operations may be performed by different networked elements in operative communication with the computing device. For example, at least one computing device 1500 may be employed in the performance of some or all of the stages disclosed with regard to the methods. Similarly, an apparatus may be employed in the performance of some or all of the stages of the methods. As such, the apparatus may comprise at least those architectural components as found in computing device 1500.

Furthermore, although the stages of the following example method are disclosed in a particular order, it should be understood that the order is disclosed for illustrative purposes only. Stages may be combined, separated, reordered, and various intermediary stages may exist. Accordingly, it should be understood that the various stages, in various embodiments, may be performed in orders that differ from the ones disclosed below. Moreover, various stages may be added or removed without altering or departing from the fundamental scope of the depicted methods and systems disclosed herein.

Consistent with embodiments of the present disclosure, a method may be performed by at least one of the modules disclosed herein. The method may be embodied as, for example, but not limited to, computer instructions which, when executed, perform the method. The method may comprise the following stages:
receiving, by a processor, a request for clinical data from a first electronic health record (EHR) system;
determining, by the processor, availability of an application programming interface (API) for accessing the clinical data;
determining, by the processor, availability of a user interface automation mechanism for accessing the clinical data;
monitoring, by the processor, real-time health metrics comprising response times and error rates for the API and the user interface automation mechanism;
selecting, by the processor, between the API and the user interface automation mechanism based on the real-time health metrics;
executing, by the processor, the request using the selected mechanism to retrieve the clinical data;
normalizing, by the processor, the retrieved clinical data into a standardized format; and
annotating, by the processor, the normalized clinical data with provenance metadata indicating the selected mechanism and retrieval timestamp.

Although the aforementioned method has been described to be performed by the platform 100, it should be understood that computing device 1500 may be used to perform the various stages of the method. Furthermore, in some embodiments, different operations may be performed by different networked elements in operative communication with computing device 1500. For example, a plurality of computing devices may be employed in the performance of some or all of the stages in the aforementioned method. Moreover, a plurality of computing devices may be configured much like a single computing device 1500. Similarly, an apparatus may be employed in the performance of some or all stages in the method. The apparatus may also be configured much like computing device 1500.

Both the foregoing overview and the following detailed description provide examples and are explanatory only. Accordingly, the foregoing overview and the following detailed description should not be considered to be restrictive. Further, features or variations may be provided to those set forth herein. For example, embodiments may be directed to various feature combinations and sub-combinations described in the detailed description.

### II. Platform Configuration

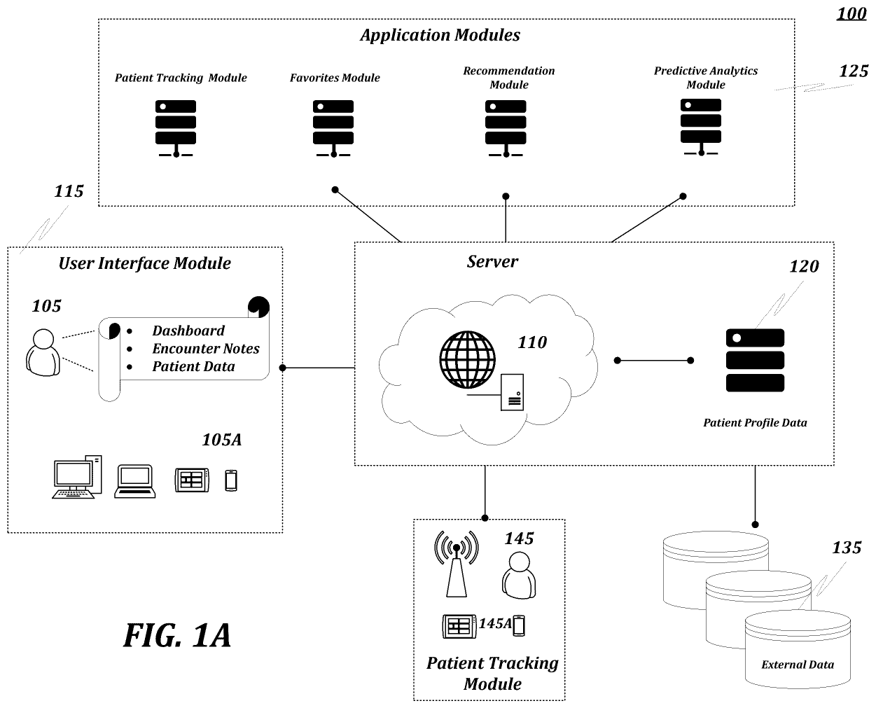

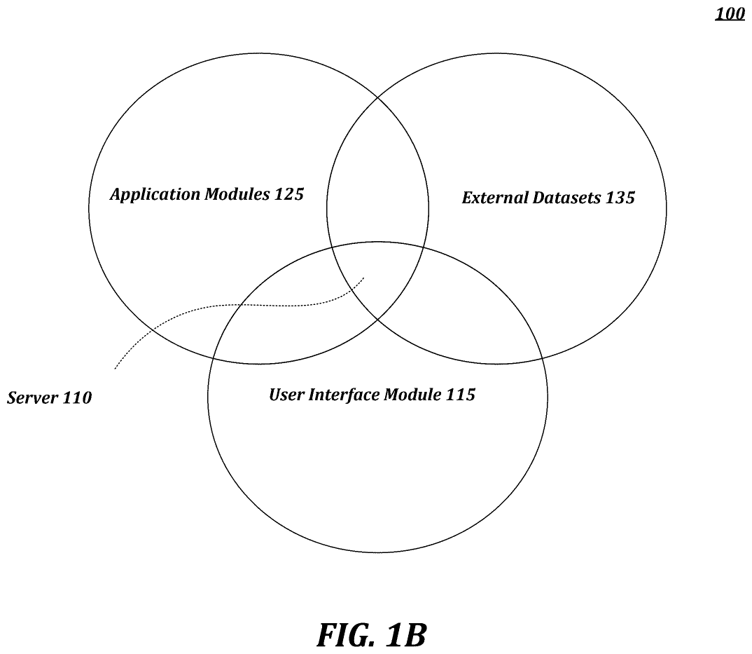

FIGS. 1A and 1B are an illustration of a platform consistent with various embodiments of the present disclosure. By way of non-limiting example, the online platform 100 for managing electronic medical records and recommending treatment plans, preventive actions, and preparedness actions based analysis of patient profile data 120 may be hosted on a centralized server 110, such as, for example, a cloud computing service. The centralized server may communicate with other network entities, such as, for example, a plurality of mobile devices, wearable devices (such as watches or smart glasses), encoding devices, electronic devices (such as desktop computers, laptop computers etc.) and one or more recording devices (e.g., camera drones, handheld camera, or installed cameras) over a communication network, such as, but not limited to, the Internet. Further, users of the platform may include, but are not limited to, practitioners (e.g., physicians), practitioner support staff (e.g., nursing assistant, patient care attendant, medical assistant, aide, medical secretary, home care aide, or other staff), and other users (e.g., patients). Accordingly, electronic devices operated by the practitioner, practitioner support staff, patient, and other users may be in communication with the platform.

A user 105, such as a practitioner, practitioner support staff, patient, or other user, may access platform 100 through a software application. The software application may be embodied as, for example, but not be limited to, a website, a web application, a desktop application, and a mobile application compatible with a computing device 1500.

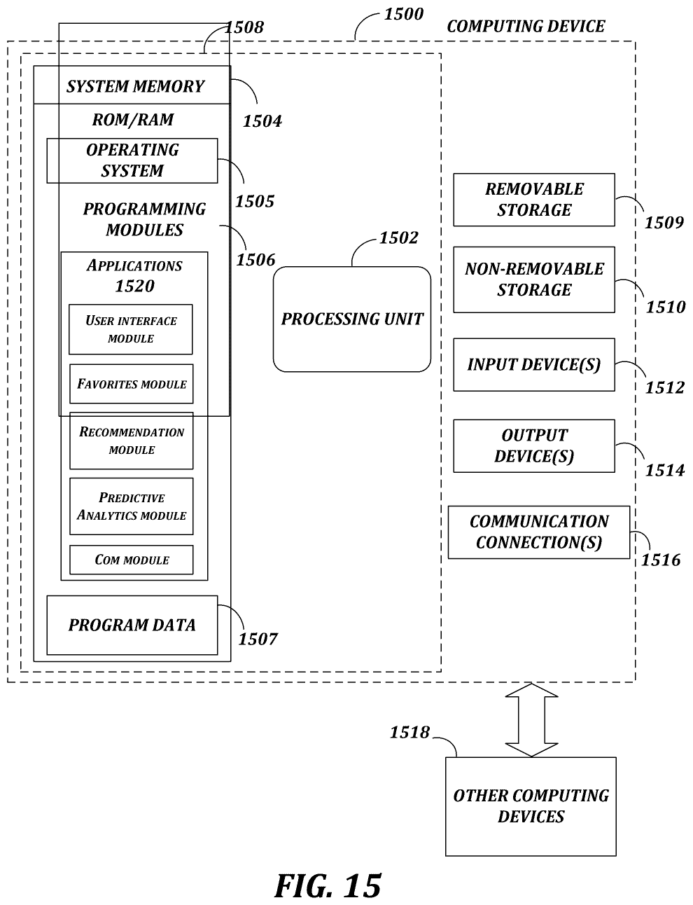

As will be detailed with reference to FIG. 15 below, the computing device through which the platform may be accessed may comprise, but not be limited to, for example, a desktop computer, laptop, a tablet, or mobile telecommunications device. Though the present disclosure is written with reference to a mobile telecommunications device, it should be understood that any computing device may be employed to provide the various embodiments disclosed herein.

The aforementioned modules may be used and interconnected in various combinations with one another. By way of non-limiting example, any of the application modules may be interconnected and/or used in conjunction with at least one of: a user interface module, a patient tracking module, external data or datasets. The application modules comprising at least one of: a favorites module, a recommendation module, a patient tracking module, a predictive analytics module, and a user interface module. The modules may be in communication with a central server. In an embodiment, the modules may reside within a computer readable medium, a server, a computer, a phone, a mobile device, a wearable device, or other electronic device.

In another embodiment, the modules can also be used together in conjunction and/or interconnected as separate elements of a system. In another embodiment, the modules may be interconnected and/or configured and/or working together as separate elements or components, while not part of or contained within the same component. In other embodiments, the modules may be contained within the same component and/or interconnected within one component or portion of a component and/or components.

In an embodiment, the module may be configured and/or used as an element and/or elements of a system. In yet other embodiments, functionality for any of the aforementioned modules can be interconnected such that the functionality is embodied in one individual module alone or collectively in a portion and or subset of the modules. The modules can be embodied as software, hardware or a combination thereof. The modules can also be collectively embodied in one device, server, computer readable medium, or hardware element.

The following is made with reference to FIGS. 1A and 1B.
I. Embodiments of the present disclosure provide a software and hardware platform comprised of a distributed set of modules, including, but not limited to:
A. User Interface Module 115;
B. Favorites Module 125; and
C. Recommendation Module 125.

In some embodiments, the present disclosure may provide an additional set of modules for further facilitating the software and hardware platform. The additional set of modules may comprise, but not be limited to:
A. Patient Tracking Module 125 and/or 145;
B. Predictive Analytics Module 125; and
C. Communications Module.

In some embodiments, the present disclosure may provide an additional set of sub-modules for further facilitating the software and hardware platform. The additional set of sub-modules may comprise, but not be limited to:
A. Preventive Care Module; and
B. Preparedness Module 125.

FIGS. 1A and 1B illustrate non-limiting examples of operating environments for the aforementioned modules. Although modules are disclosed with specific functionality, it should be understood that functionality may be shared between modules, with some functions split between modules, while other functions duplicated by the modules. Furthermore, the name of the module should not be construed as limiting upon the functionality of the module. Moreover, each stage in the claim language can be considered independently without the context of the other stages. Each stage may contain language defined in other portions of this specification. Each stage disclosed for one module may be mixed with the operational stages of another module. Each stage can be claimed on its own and/or interchangeably with other stages of other modules.

FIG. 1A Description: Still consistent with embodiments of the present disclosure, platform 100 may be configured to access data from various external databases 135. External databases 135 may comprise, but not be limited to, for example medical resources. The data may be analyzed by platform 100 in order to facilitate a plurality of functions and features within platform 100.

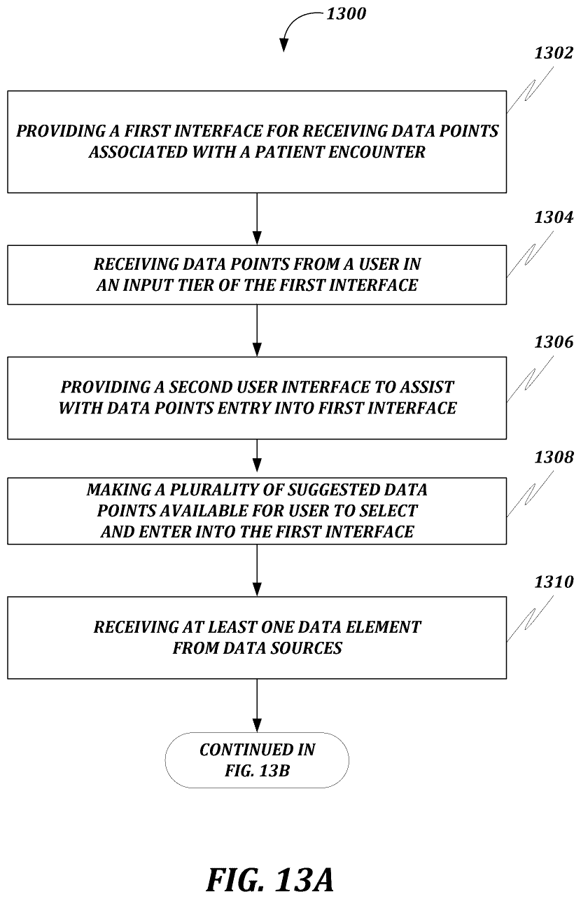

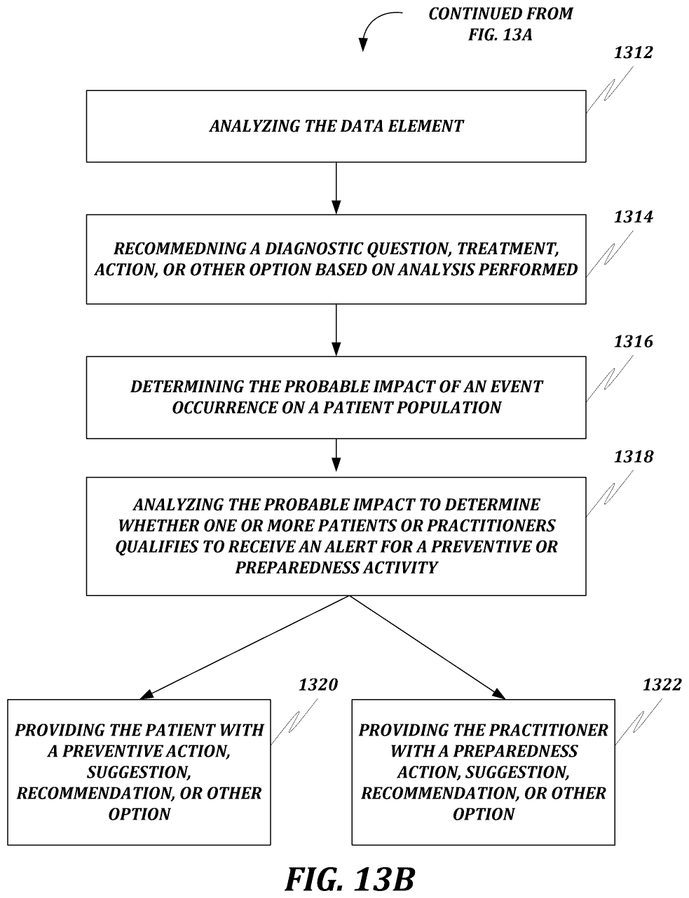

FIG. 1B shows a Venn Diagram illustrating the connections between the various components of platform 100. As can be deduced, UI module 115 may have display to user 105 to external datasets for a real time view of the data being communicated to platform 100. Application Modules 125 may further access external datasets 135 for real time, actionable data. UI module 115 may be further enabled to receive updates via Application Modules 125. In some embodiments, server 110 may be configured as an intermediary between each platform component, while in other embodiments, each platform component may communicate directly, without server 110 as an intermediary.
II. Embodiments of the present disclosure provide a software and hardware platform comprised of a distributed set of computing elements, including, but not limited to:
A. A Computing Device

Wherein the platform is operative to control a computing device in furtherance of the operation of the application modules,
The computing device comprising, but not limited to at least one of the following:
   a processing unit,
   a memory storage,
Wherein the computing device may be embodied as a mobile computing device,
   wherein the mobile computing device comprises, but is not limited to,
      a tablet (105A),
      a smartphone (105A),
      a drone,
      a wearable camera (105A),
      a handheld camera (105A),
      an installed camera (105A), and
      a remotely operable recording device;

Wherein the computing device is configured to perform a method comprising:
an input method comprising: (FIG. 13A, 1300);
   providing a first interface for receiving data points associated with a patient encounter (FIG. 13A, 1302), wherein the first interface comprises:
      an input tier for receiving data points from a user (FIG. 13A, 1304); and
   providing a second interface for assistant with an entry of the data points into the first interface (FIG. 13A, 1306), wherein the second interface comprises:
   a plurality of suggested data points made available for user selection, wherein at least one selected suggested data point is configured to populate a currently selected input tier of the first interface (FIG. 13A, 1308); and
   an improved patient diagnostics method comprising:
   receiving at least one data element associated with at least one of the following:
      a plurality of patient data, and
      at least one data point in a patient encounter note during a patient diagnostic process (FIG. 13A, 1310);
      analyzing the data element (FIG. 13B, 1312); an input assistance method comprising:
   recommending, based on the analysis, at least one of the following:
      a diagnostic question,
      a recommended treatment, and
      a recommended action (FIG. 13B, 1314); an analytics method comprising:
   determining, based on the analysis, whether an occurrence of an event may cause an adverse effect on a patient (FIG. 13B, 1316);
      predicting, based on the analysis, at least one of the following:
         a preventive suggestion, and
         a preventive action, and
         a preemptive preparedness suggestion, and
         a preemptive preparedness action (FIG. 13B, 1318);
an action generating method comprising:
   providing the patient or practitioner with at least one of the following:
      the preventive suggestion, and
      the preventive action (FIG. 13B, 1320), and
      the preemptive preparedness suggestion, and
      the preemptive preparedness action (FIG. 13B, 1322).

Wherein the computing device may be embodied as any of the computing elements illustrated in FIG. 1A, including, but not limited to, Application Modules 125, Patient Tracking Module 145, User Interface Module 115; and Server 110.

Various hardware components may be used at the various stages of operations follow the method and computer-readable medium. For example, although the methods have been described to be performed by a computing device, it should be understood that, in some embodiments, different operations may be performed by different networked elements in operative communication with the computing device. For example, server 110 and/or computing device 1500 may be employed in the performance of some or all of the stages disclosed with regard to the methods below.

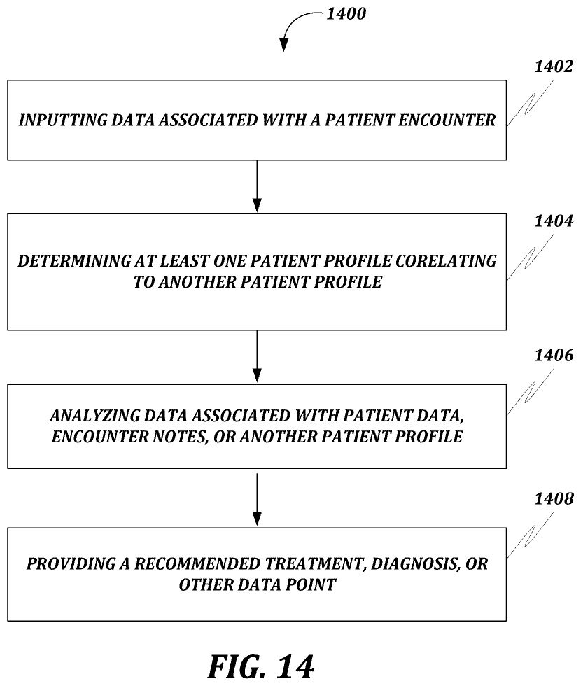

A. Sub-Modules Associated with the Computing Device
Platform may be operative to control at least one of the following sub-modules of a computing device:
   a user interface module,
   a recommendation module,
   a favorites module,
   a predictive analytics module,
   a patient tracking module,
   a preventative care module, and
   a preparedness module.
1. The User Interface Module
   a. Enables user-control of the Computing Device
   b. Enables user-control of the Sub-Modules of the Computing Device:
      i. The user interface module
      ii. The recommendation module
      iii. The favorites module
      iv. The patient tracking module
      v. The predictive analytics module
      vi. The preventative care module
      vii. The preparedness module
      viii. Communications module
   c. Enables user-control of the Platform Modules:
      i. The recommendation module
      ii. The favorites module
      iii. The patient tracking module
      iv. The predictive analytics module
      v. The preventative care module
      vi. The preparedness module
   d. Enables operative control of input medical information hardware during a patient encounter:
      i. Input Device (FIG. 14, 1402)
         1. Keyboard
         2. Mouse
         3. Touch Screen/Touch Pad
   e. Enables capturing based on data:
      i. Recordation of content received from the communications module connected to Input Device
      ii. Recordation of media captured during patient encounter on the computing device (e.g., microphone, audio, video recording)
2. The Favorites Module
   a. Enables Analysis of frequency of captured and stored content:
      i. Enables Frequency Mapping based on, but not limited to, patient data and temporal parameters
      ii. Enables sorting of patient data to determine correlations and frequency (FIG. 14, 1404)
3. The Patient Tracking Module
   a. Operative control of a location associated with the computing device
   b. In operative communication with a central server, GPS, or location based sensors
   c. Time stamps and location coordinates captured by the patient tracking module
   d. Used for syncing and analysis from various data sources
   e. Enables the reading and communicating of location data associated with a sensing device
   f. The location data may be obtained by way of, for example, but not limited to:
      i. GPS/IP Address/Triangulation
      ii. LAN/WAN
4. The Recommendation Module
   a. Enables the refinement of treatment plans, initiatives, and other suggested courses of action based on analysis of data sources
   b. More effective as more relevant and correlated patient data is made available by data sources (FIG. 14, 1408)
5. The Predictive Analysis Module
   a. Enables predictive analysis for patient preventive care
   b. Enables predictive analysis for practitioner preparedness
   c. The Preventive Care Module
   d. The Preparedness Module
6. The Communications Module
   a. Enables the networking of the multiple application modules associated with multiple networked devices or singular communication device or server
   b. In operative communication with other communications modules of computing devices
   c. Configured to communicate with nearby devices also running on the platform
   d. Configured to join ‘groups’of devices analyzing data under a similar location, theme, or other type of association
   e. Remote control of the capturing modules
   f. Remote control of the camera
   g. Remote control of the microphone
   h. Via Wireless Media
   i. Via Wired Media

Embodiments of the present disclosure provide a hardware and software platform operative by a set of methods and computer-readable media comprising instructions configured to operate the aforementioned modules and computing elements in accordance with the methods.

The methods and computer-readable media may comprise a set of instructions which when executed are configured to enable a method for inter-operating at least one of the following modules:
I. User Interface Module
II. Favorites Module
III. Recommendation Module
IV. Predictive Analytics Module
   a. Preventative Care Sub-Module
   b. Preparedness Sub-Module

The aforementioned modules may be inter-operated to perform a method comprising the following stages:

A User Interface Module comprising:
at least two stages of assisted data entry, the input system comprising:
a first stage of input for receiving data points from a patient;
a second stage of input for receiving treatment notes for the patient, and
wherein the first stage of input comprises a plurality of tiers for specifying the data points for the patient, the plurality of tiers comprising at least one of the following:
a first tier for specifying at least one of the following: reasons for visit/chief complaints;
   a second tier for specifying at least one of the following: History of Reason for visit/History of Present Illness (HPI);
   a third tier for specifying a body system associated with at least one of the following: the first tier and the second tier;
   a fourth tier for specifying a condition associated with the body system specified in the third tier;
wherein the second stage of input comprises a plurality of tiers for specifying the treatment notes for the patient, the plurality of tiers comprising at least one of the following:
   a fifth tier for specifying a treatment plan; and
   a sixth tier for specifying a treatment in accordance to the treatment plan.

Variation: Wherein the first stage of input and the second stage of input are compiled into a patient encounter note.

Variation: Wherein a completion of the patient encounter note is facilitated, at least in part, by at least one of the following:
a. A favorites module;
b. A recommendation module; and
c. An analytics module.

A Favorites Module comprising:
providing a first interface for receiving data points associated with a patient encounter, wherein the first interface comprises:
   an input tier for receiving data points from a user; and
providing a second interface for assistant with an entry of the data points into the first interface, wherein the second interface comprises:
a plurality of suggested data points made available for user selection, wherein at least one selected suggested data point is configured to populate a currently selected input tier of the first interface; and

A Recommendation Module comprising:
receiving at least one data element associated with at least one of the following:
   a plurality of patient data, and
   at least one data point in a patient encounter note during a patient diagnostic process;
analyzing the data element;
recommending, based on the analysis, at least one of the following:
   a diagnostic question,
   a recommended treatment, and
   a recommended action;

Variation: wherein the patient diagnostic process is configured to guide a user through inputting data associated with a patient encounter, and
wherein a recommendation is provided in furtherance of completing the patient diagnostic process;

Variation: determining at least one patient profile similar to the patient profile associated with the patient encounter note;
analyzing data points within at least one patient encounter note associated with the at least one similar patient profile; and
determining a recommendation based on the analysis of the patient encounter note and the at least one patient encounter note associated with the at least one similar patient profile.

Variation: wherein analyzing data points within the at least one patient encounter note associated with the at least one similar patient profile comprises analyzing at least one of the following:
previous encounters for each patient profile;
previous diagnosis for each patient profile; and
previous treatments for each patient profile.

A Predictive Analytics Module comprising:
determining, based on the analysis, whether an occurrence of an event may cause an adverse effect on a patient;
predicting, based on the analysis, at least one of the following:
   a preventive suggestion,
   a preventive action,
   a preemptive preparedness suggestion, and
   a preemptive preparedness action; an action generating method comprising:
providing the patient or practitioner with at least one of the following:
   the preventive suggestion,
the preventive action,
the preemptive preparedness suggestion, and
the preemptive preparedness action.

Variation: receiving at least one data element associated with at least one of:
a plurality of patient data,
a plurality of external data,
a plurality of patient profile data,
a plurality of encounter notes,
a plurality of patient tracking data,
other data;
analyzing patient data for a plurality of patients;
determining whether one or more practitioners qualifies to receive an alert; and
providing an alert to the one or more practitioners.

Variation: determining whether one or more patients qualifies to receive an alert; and
providing an alert to the one or more patients.

Variation: wherein analyzing further comprises:
determining at least one patient profile similar to the patient profile associated with a set of conditions;
analyzing data points within at least one patient data element associated with at least one determined set of conditions; and
determining a preemptive preparedness action based on the analysis of the patient data element and the at least one external data element associated with at least one condition.

Variation: further comprising:
wherein determining further comprises determining whether there is a high probability that a known set of conditions will adversely impact one or more patients; and
wherein analyzing further comprises determining whether a probability of an occurrence and event has reached a predetermined threshold; and
wherein determining further comprises a positive correlation between an external patient data element meeting the predetermined threshold and a patient data element triggers an alert.

Variation: further comprising:
wherein the occurrence of an event is at least one of: high pollen count, poor air quality, high smog level, high lead level in public water supply, poisonous gas released, high radiation levels, terror attack, fire, flooding, earthquake, tornado, heat wave, drought, an FDA issued warning against a drug that is used by the patient, a possible food contamination (e.g., salmonella warning, etc.), or other emergent event.

Although the stages are disclosed in a particular order, it should be understood that the order is disclosed for illustrative purposes only. Stages may be combined, separated, reordered, and various intermediary stages may exist. Accordingly, it should be understood that the various stages, in various embodiments, may be performed in arrangements that differ from the ones claimed below. Moreover, various stages may be added or removed from the without altering or deterring from the fundamental scope of the depicted methods and systems disclosed herein.

Embodiments of the present disclosure provide a hardware and software platform operative as a distributed system of modules and computing elements.

### User Interface Module

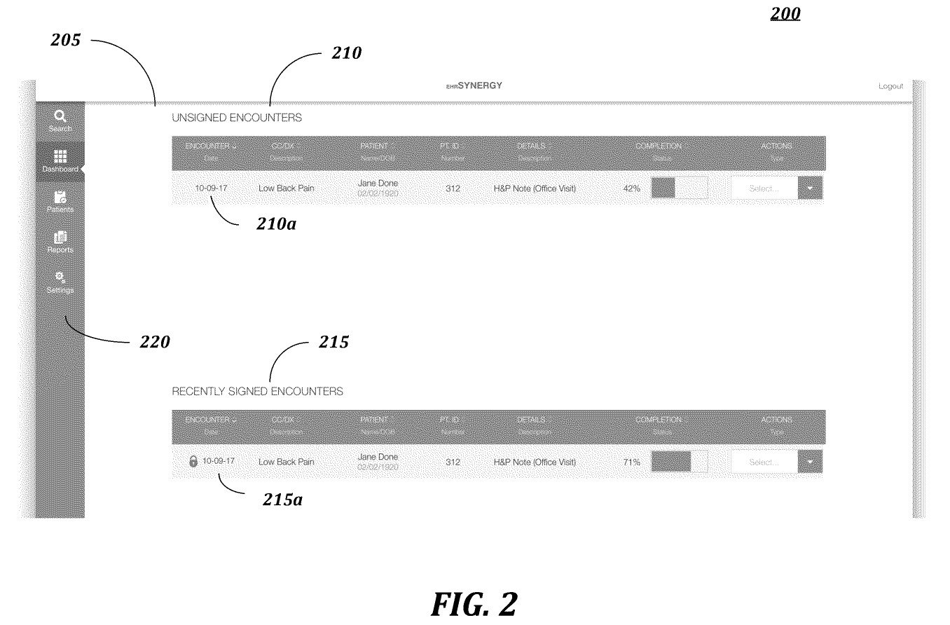

FIG. 2 illustrates a dashboard 200 in accordance to some embodiments of the present disclosure. Dashboard 200 may comprise a main segment 205 and a side panel 220. Side panel 220 may persist throughout the various views of platform 100, and enable user navigation to different platform segments. Main segment 205 may comprise data associated with the dashboard 200, as selected in side panel 230. In some embodiments, dashboard 200 may display patient visit data, which may be referred to as ‘encounters’. Encounters may be segmented by an unsigned encounters view 210 (e.g., incomplete encounters or drafts), comprising line-item encounters 210a, and a recently signed encounters view 215, comprising line-item encounters 210b. Once an encounter note is complete, it may be signed and locked from editing by a provider.

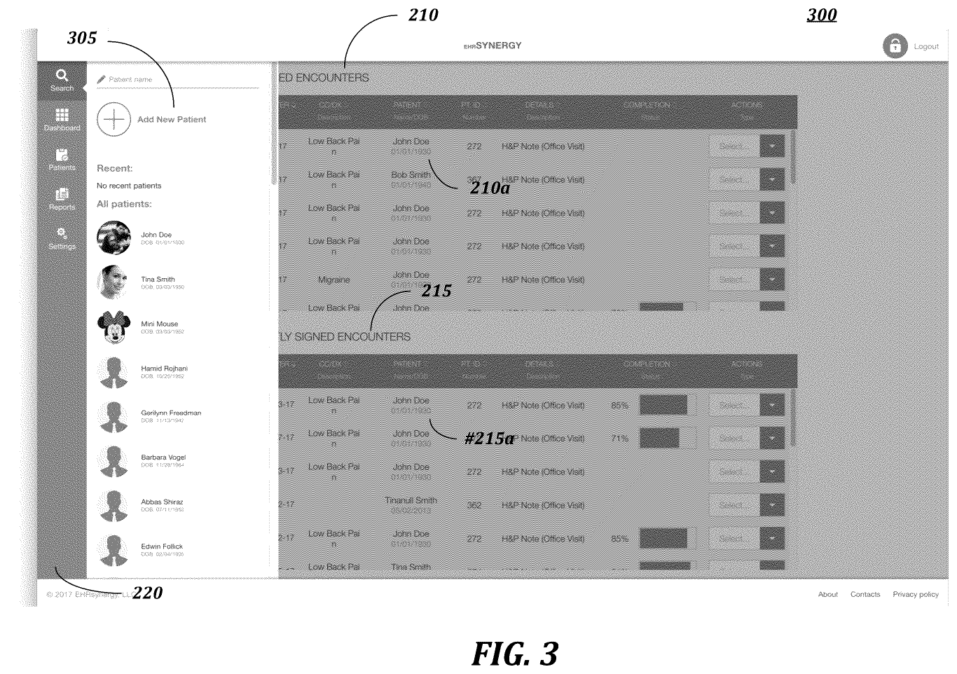

FIG. 3 illustrates an expanded side panel view 300 in accordance to some embodiments of the present disclosure. Side panel view 300 may be triggered upon, for example, a selection of a selectable button in side panel 220. The selectable button may provide an expanded set of options extending from side panel 220, including, but not limited to, for example, adding a new patient selector 305, which, upon selection, may enable the entry of new patent data into platform 100. In some embodiments, recent patient data may be displayed in the expanded side panel 300, as well as a full listing of patient data.

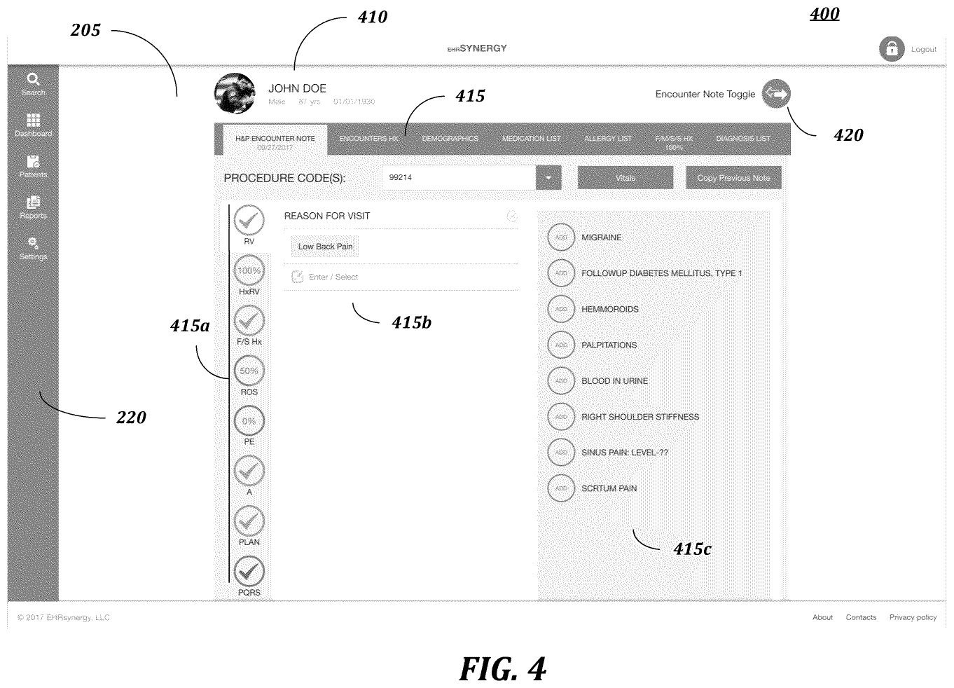

FIG. 4 illustrates an encounter view 400 in accordance to some embodiments of the present disclosure. Encounter view 400 may be presented within main segment 205. An encounter may be related to a patient profile 410. A full profile view may be toggled by encounter note toggle 420. As such, a patient may be associated with a plurality of encounters and platform 100 may be configured to cross reference the plurality of encounters to derive data for the various embodiments disclosed herein. Encounter view 400 may comprise a plurality of tabs 415. Each tab may enable a user 105 to specify a plurality of encounter data. For example, the encounter history tab may display encounter history data for a selected patient, the demographics tab may display patient profile data (e.g., race/ethnicity, and the like), the medication list and allergy list tab may specific active and inactive medications and allergies, respectively.

In some tabs, main segment 205 may further be sub-divided by a input tier panel 415a, which may illustrate the corresponding input tiers for each stage of data entry. FIG. 4 illustrates the specification of, for example, reason for visit input tier 415b. User 105 may select from favorites module view 415c, or favorites module 125. In accordance to the various embodiments herein, each input tier may comprise a favorites module view 415c, which is associated with the underlying processing of the favorites module 125. Thus, the favorites module view 415c may list suggested, frequent, or recommended inputs for each selected tier by invoking the functionality of the favorites module 125. Details with regard to favorites module 125 functionality are disclosed in the subsequent sections of the present disclosure.

As the user completes each input tier, a percentage indicator may appear within the input tier panel, indicating to user 105 whether or not they have completed the necessary inputs for the corresponding tier. Once complete, a complete indicator may appear such as, for example, but not limited to, a check mark. Employing favorites module view 415c enables user 105 to more efficiently and effectively input data, as it provides intelligent suggestions to augment the input process for this tier.

In the illustrated example, user 105 begins specifying the encounter by inputting John Doe's Reason for Visit into the input tier 415a and is assisted with suggested input items by favorites module view 415c. In some embodiments, the reasons for visit may be referred to as “Chief Complaints.” Further still, and as will be detailed below, certain indicators may appear complete upon the creation of the encounter note, as the data used to complete the corresponding inputs may be retrieved from, for example, a pre-populated patent profile.

The system may be configured to enter the reasons for visit in the patient's own words instead of just the results of the physical exam based on the physician or practitioner.

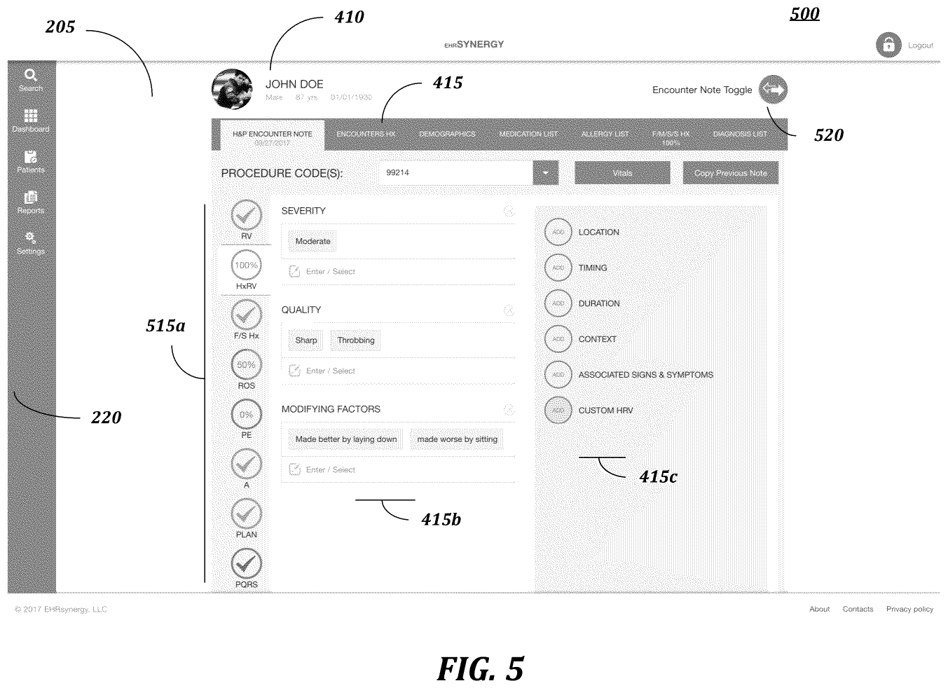

Turning now to FIG. 5, a History of Reason for Visit input tier 515a is illustrated within encounter input view 500. Continuing the illustrated Example, user 105 may now proceed, via subsequent selection in the input tier panel 515a, to inputting John Doe's symptom history as it relates to the reason for visit (may also be referred to as History of Present Illness (HPI) in some embodiments) as illustrated in view 500.

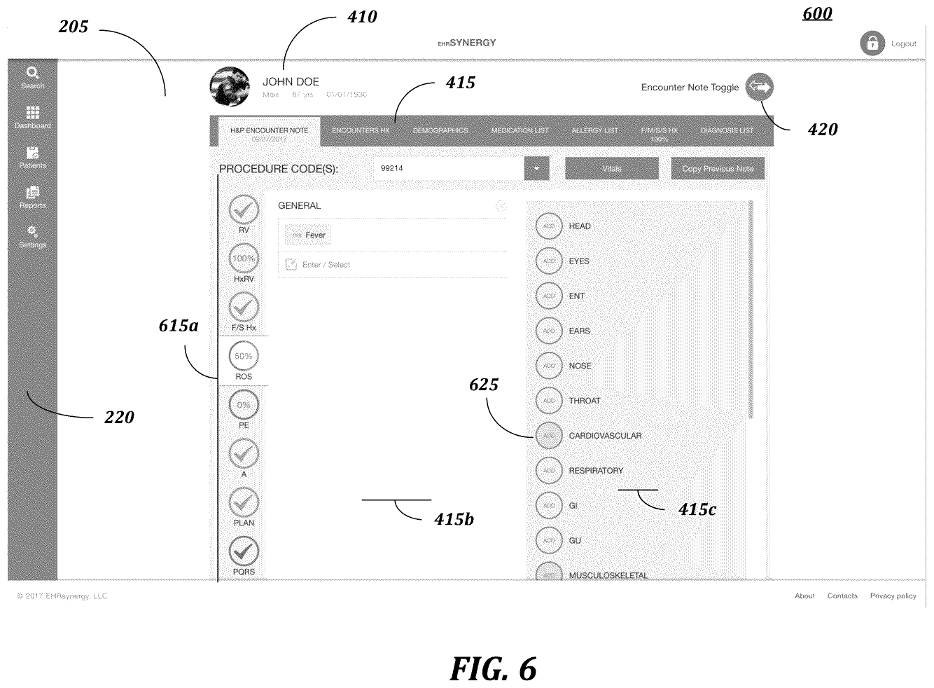

FIG. 6 illustrates a Review of Symptoms (ROS) input tier 615a in accordance to some embodiments of the present disclosure. Continuing the illustrated Example, user 105 may now proceed, via subsequent selection in the input tier panel 515a, to inputting John Doe's symptoms as it is determined by the examiner as illustrated in view 600.

Referring still to FIG. 6, favorites module view 415a may provide user 105 not only with a list of symptoms that may be selected, but also with color coded indicators 625, indicating the likelihood and/or frequency with which the suggested symptom might occur as they relate to the other inputs in the encounter note and related encounter notes. The color coded elements may be blue, yellow, or green indicating varying frequencies. Other colors may be used for other indications such as red, orange, and other colors.

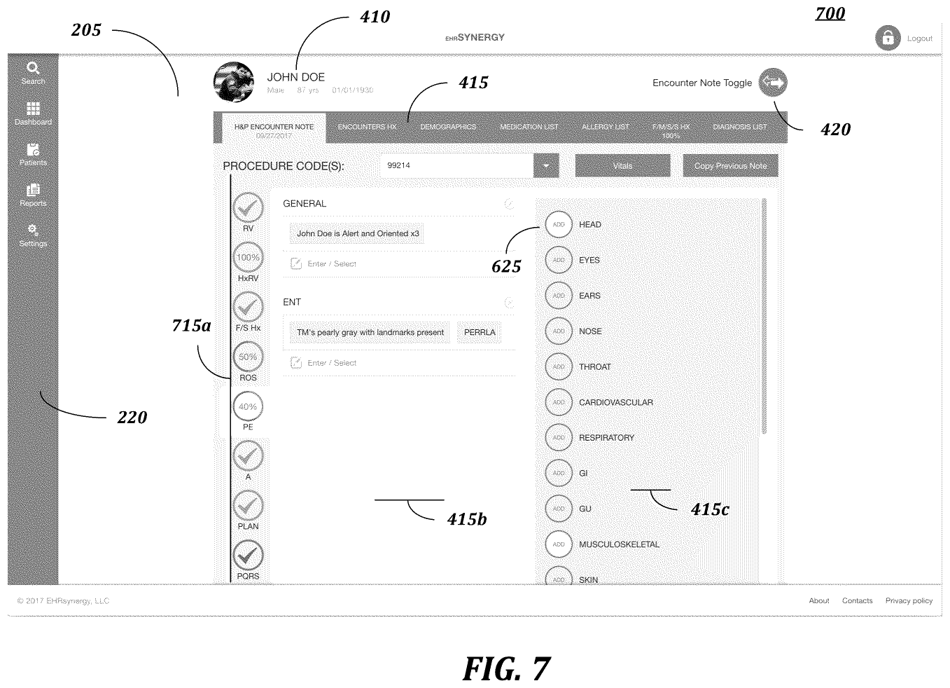

Turning now to FIG. 7, a physical exam input tier 715a may be provided in various embodiments of the present disclosure. Continuing the illustrated Example, user 105 may now proceed, via subsequent selection in the input tier panel 715a, to inputting John Doe's symptoms as it is determined by the examiner as illustrated in view 700. Whereas ROS may be based on the subjective responses of a patient, the PE may be based on a physical exam by a provider.

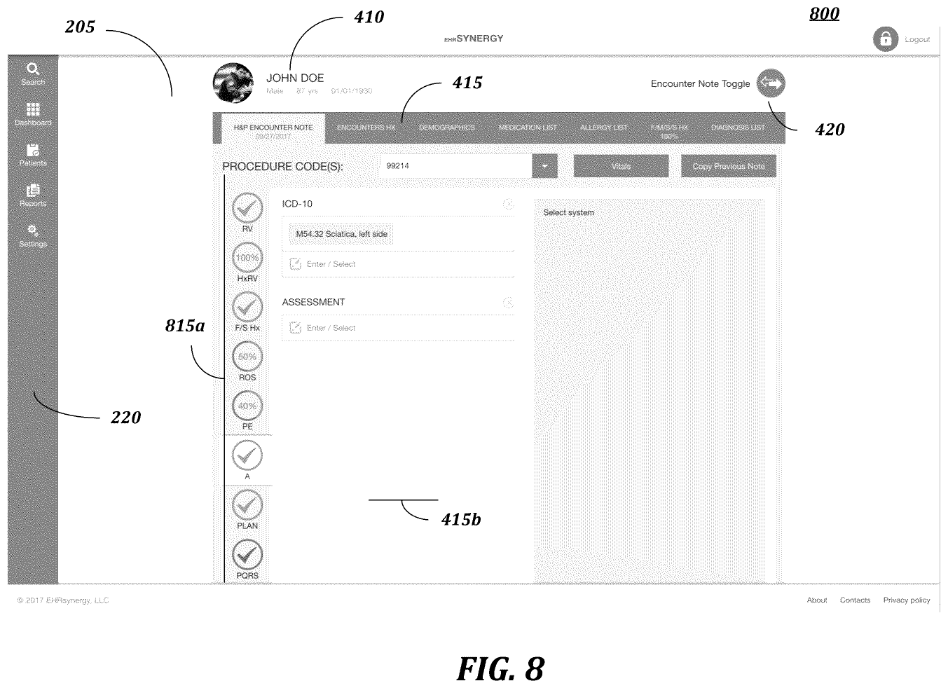

FIG. 8 illustrates an assessment input tier 815a in accordance to some embodiments of the present disclosure. Continuing the illustrated Example, user 105 may now proceed, via subsequent selection in the input tier panel 815a, to inputting an assessment for John Doe's as it is determined by the examiner as illustrated in view 800.

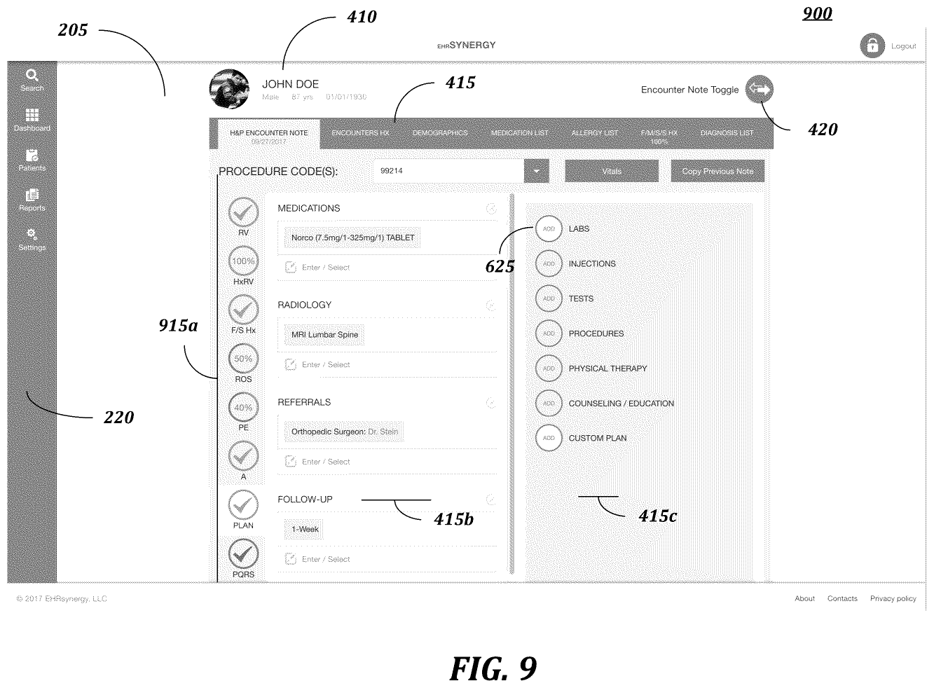

Having inputted an assessment, platform 100 may enable user 105 to input a treatment plan. FIG. 9 illustrates a treatment plan input tier 915a in accordance to some embodiments of the present disclosure. Continuing the illustrated Example, user 105 may now proceed, via subsequent selection in the input tier panel 915a, to inputting a treatment for John Doe's as it is determined by the examiner as illustrated in view 900.

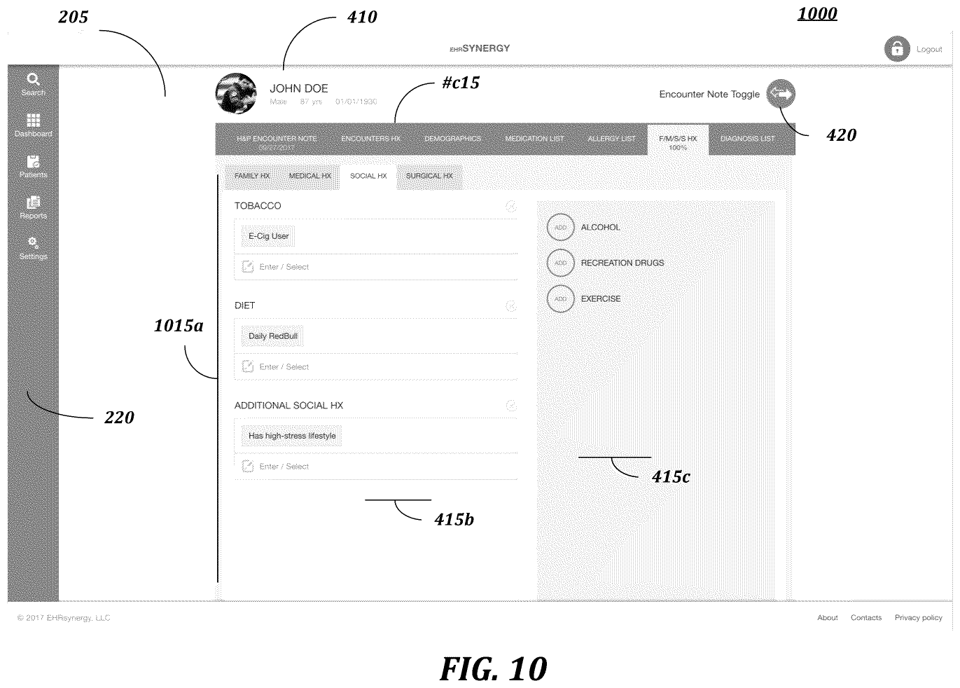

FIG. 10 illustrates a family and patient history view 1000 in accordance to the embodiments of the present disclosure. View 1000 provides user 105 with data points or data elements associated with the patient at hand, their family history, medical history, social history, and surgical history. These data points, data, or data elements may be used when creating a new encounter for the patient. Thus, data from the family and personal history view 1015a may be inputted and pulled into subsequent encounter notes for the patient. Moreover, the data points may remain up-to-date with every new encounter for the patient, as well as related patients in order to maintain an accurate family history view.

Unlike the other input tiers that specify data for a particular encounter, family and patient history data may be kept consistent across all encounters, as well as the other elements of tabs 415 such as, for example, but not limited to, encounter history, demographics, medication list, allergy list, and diagnosis list. In the illustrated example, user 105 begins specifying the encounter by inputting historical data into the input tier 1015a, and is assisted with suggested input items by favorites module view 415c. The inputted data may, in turn, persist across all encounters associated with John Doe. Accordingly, upon the creation of the encounter, a completion indicator may appear for data which has already been pulled from a pre-existing entry.

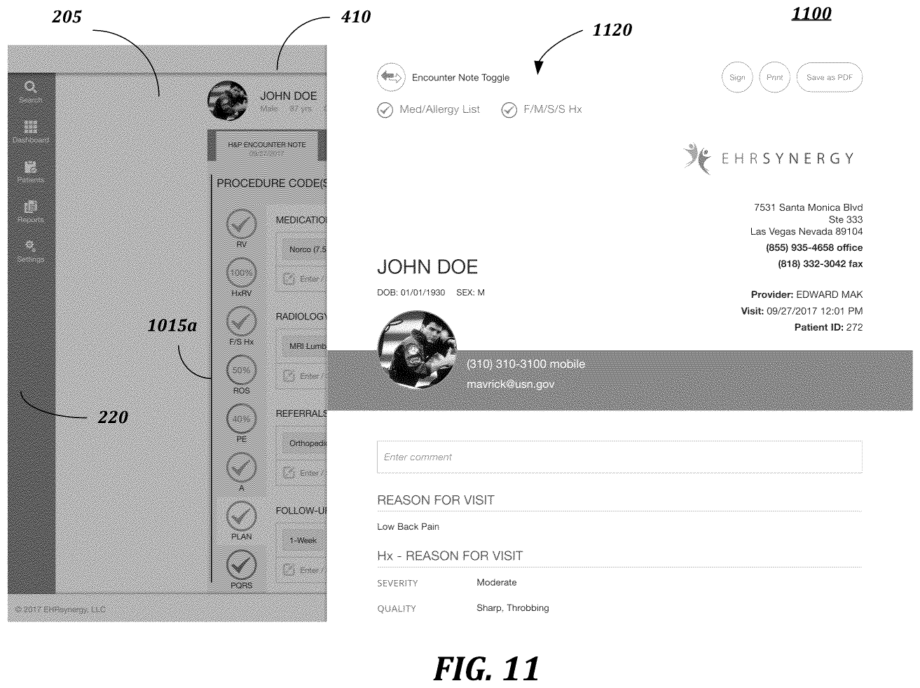

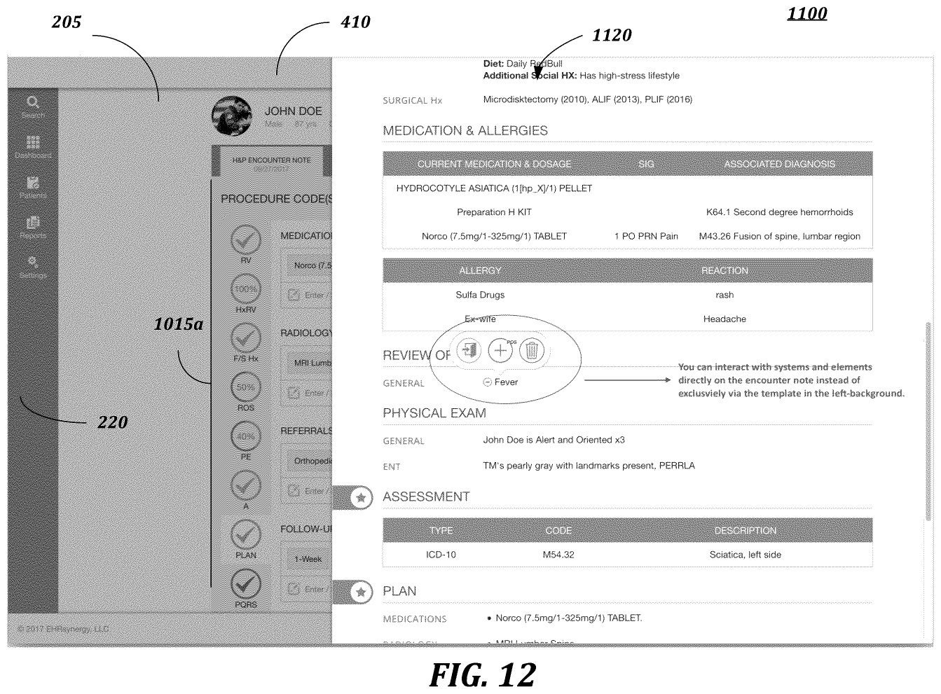

FIGS. 11 and 12 illustrate an encounter note view 1100 in accordance to some embodiments of the present disclosure. Appendix A provides a full view of an example of an encounter note. Encounter note view 1100 can be engaged by user 105's selection of the encounter note toggle 420. Encounter note 1100 provides an overview 1120 of the inputs made and retrieved throughout the various input tiers. However, unlike the main segment view 410, overview 1120 enables the user to review and edit the various data points within the encounter note without the rigidity of the guided template-like inputs of the main segment view 410. The toggle note may further provide functionality to edit any input data, copy data from a previous encounter, or delete data. For example, in 12, the first button can navigate a user to that specific tier. The second button can edit and/or modify the encounter note. The third button may delete the encounter note. In 12, a user may copy a previous visit, previous encounter note, or other data entry point.

### Favorites Module

Embodiments of the present disclosure may provide a module for displaying, for user selection, standardized inputs that are frequently inputted for a corresponding input tier. These displayed inputs made for user selection may be referred to as ‘favorites’ and made available to the user in a ‘favorites view’ or ‘favorites module’ that accompanies the input tiers.

For example, where user 105 loads a patient encounter note and enters a first input tier, such as the ‘reasons for visit’ input tier, the favorites module may be configured to recommend data point, data, or data elements to be used for inputs. As will be detailed below, the recommended data points, data, or data elements may be, for example, previously used data points, data, or data elements. The user may simply select one or more of the recommended data points, from the favorites module, and the selected data points will be populated into the corresponding input tier.

First Type: Helps with Data Point Categories Within an Input Tier

In yet further embodiments of the present disclosure, the favorites module may be configured to assist the user in inputting commonly used data point, data, or data elements categories. Referring to FIG. 5 illustrates one example, wherein user 105 is within the history of reasons for visit input tier. Some common data point categories that user 105 may enter with regard to this input tier may include, for example, severity, quality, and modifying factors.

As user 105 inputs the data points associated with such categories, favorites module 415c may further suggest to the user additional data point, data, or data elements categories that may be valuable for user 105 to further specify, such as, for example, for not limited to: location, timing, duration, context, and associated signs & symptoms. In this way, as user 105 completes the patient encounter note, favorites module 415c may be configures so as to enable user 105 to quickly and effectively ensure that the input tier is fully and sufficiently specified.

FIG. 9 illustrates yet another example of favorites module 415c providing recommended categories of input tiers. In this instance, favorites module 415c may provide recommendations of data point, data, or data elements categories in a second stage of input tiers, in which the treatment portions of the patient's encounter note is specified.

Favorites module 125 may be enabled to determine the categories of data points for a given input tier based on, for example, but not limited to, historical data. In some embodiments, favorites module 125 may be configured to analyze the various categories of data points that various users of platform 100 have entered for various patient encounter notes. The analysis may then determine which data point categories are frequently used in conjunction with corresponding input tiers. In this way, favorites module 415c may be configured to comprise frequently used data point categories for each input tier.

Second Type: Helps with actual data points within the Input Tier Embodiments of the present disclosure may enable a plurality of methods for determining favorite data points, data, or data elements made available for user selection in the favorites module. For example, some data points made for selection in the favorites module may be based on those data points that have already been entered by the user in previous input tiers or a currently selected input tier.

Referring to FIG. 6, wherein user 105 is completing an input tier for specifying a body system associated with a patient's symptoms, favorites module 415c may display standardized inputs for body systems commonly entered along with the inputs made in the completed input tiers. Accordingly, favorites module 415c may be configured to analyze user inputs made in completed input tiers (e.g., the ‘reasons for visit’ input tier) and analyze one or more internal and/or external datasets in order to determine those body systems that frequently accompany the inputs made in completed tiers.

### Favorites Module Analysis

Consistent with embodiments of the present disclosure, favorites module 125 may be configured to suggest data points (e.g., elements) or data point categories (e.g., systems) in accordance to a plurality of methods, and through a plurality of systems. Some of those methods and systems include, but are not limited to, for example:
Analysis performed on previous inputs for a current patient encounter note;
Analysis performed on previous patient encounter notes for the same patient;
Analysis performed on previous patient encounter notes of a plurality of patients;
   Wherein the plurality of patient encounter data is accessed through a dataset of encounter note data internal to platform 100;
   Wherein the plurality of patient encounter data is accessed through a dataset of encounter note data external to platform 100.

The aforementioned analyses may be based on, at least in part, a determination of which data points or data point categories frequently occur together within an encounter note and/or within an input tier of an encounter note. In this way, favorites module 125 may determine which data points or data point categories to display within favorites module 415c.

In some embodiments, data read from external databases 135 may be used to facilitate recommendations in a favorites module 125. In turn, favorites module 125 may have access to patient data from a plurality of different practices in order to, for example, perform its analysis so as to facilitate a determination of data point or data point category frequency. In this way, favorites module may suggest data points, data, or data elements for selection in a first input stage, as well as recommendations for treatment and treatment plans in a second input stage. It should be understood that data may be anonymized and presented in a way so as to be HIPPA compliant.

Still consistent with embodiments of the present disclosure, favorites module 125 may determine a frequency rate that certain data points or data point categories appear in correlation with an encounter note and/or an input tier within an encounter note. This frequency rate may, in turn, be indicated to user 105 through the user interface module. For example, in some embodiments, a color-coded indicator may be displayed in favorites module 415c next to each suggested data point or data point category.

One such example is illustrated in FIG. 6, with reference to element 625. In this example, favorites module 415c provides a color-coded indicator 625 next to each suggested data point. The magnitude or wave-length of the color may be correlated with a frequency with which the data point is used, thereby attracting user 105's attention in selecting the data point. In this way, user 105 may be assisted in efficiently and effectively completing the patient encounter note.

### Recommendation Module

In certain embodiments, platform 100 may comprise a recommendation module. The recommendation module may suggest data points or data point categories for various input tiers of an encounter, in a similar way to the favorites module's operation. In further embodiments, however, the recommendation module may be used to facilitate an improved diagnostic experience for user 105 and a patient that is the subject of the patient encounter. It should be noted that, throughout the various embodiments disclosed herein, recommendation module need not be used in conjunction with other components of platform 100 (e.g., UI module, an encounter note, favorites module, or analytics module). Rather, the disclosed features and functions of the recommendation module may be employed independently of other modules in platform 100.

First Type: Without External Data

Consistent with embodiments of the recommendation module may have access to internal data 120, including, but not limited to, for example, patient data and encounter note data. The recommendation module may employ internal data 120 to further use to assist user 105 in interacting with the patient.

As one example, when analyzing a patient encounter's reasons for visit and other data points entered throughout the various tiers of an encounter note, the recommendation module may suggest that the practitioner ask some diagnostic questions to the patient and input those responses into the system.

As another example, platform 100 may correlate user 105 inputs for one encounter note with those inputs made in other encounters on patients with similar patient profiles where certain new treatments were tried and have been proven to be effective.

In some embodiments, user 105 may be directed to provide additional data points addressing the recommendation module's diagnostic recommendations. These additional data points may then, in turn, be further analyzed by user 105 to, for example, ask additional diagnostic questions, provide recommended treatments, or recommend certain actions. In this way, whereas the favorites module may be used to assist user 105 with data point entry, the recommendation module may be used to user 105 with patient diagnostic and treatment.

Second Type: With External Data

By means of external datasets 135, platform 100 may be in operative communication with, for example, but not limited to, Medical Encyclopedias, WHO databases, and up-to-date diagnostics from various other data sources (i.e., weather sensors, air quality sensors, water sensors, and the like).

The external data analyzed from such external dataset sources can be used to assist user 105 throughout the various input tiers of an encounter note and/or the patient diagnostic process. These additional data points, data, or data elements may then, in turn, be further analyzed to, for example, ask additional diagnostic questions, provide recommended treatments, or recommend user 105 take certain actions.

As on example, based at least in part on data read from external sources, the recommendation module may provide recommended actions to user 105 (e.g., “quarantine patient immediately”). As another example, platform 100 may review inputs (e.g., reasons for visit, history) and find that certain new treatments have been proven to be effective for similar inputs, with similar patient profiles. In turn, these treatments may be recommended to user 105.

### Recommendation Module Analysis

Consistent with embodiments of the present disclosure, recommendation module may be configured determine suggest data points, data, data elements or data point categories in accordance to a plurality of methods, and through a plurality of systems. Some of those methods and systems include, but are not limited to, for example:
Analysis performed on previous inputs for a current patient encounter note;
Analysis performed on previous patient encounter notes for the same patient;
Analysis performed on previous patient encounter notes of a plurality of patients;
   Wherein the plurality of patient encounter data is accessed through a dataset of encounter note data internal to platform 100;
   Wherein the plurality of patient encounter data is accessed through a dataset of encounter note data external to platform 100.

In certain embodiments, the recommendation module may work in conjunction with favorites module. For instance, whereas the favorites module is used to assist in data point, data, or data elements entry, the recommendation module may be used to perform additional analysis based on external data and provide the favorites module 415a with suggested data points, data, or data elements or data point categories of inputs. In this way, favorites module 415a may now be enabled to derive data points data, or data elements for suggested entry beyond a mere frequency of data point, data, or data elements occurrence analysis.

### Predictive Analytics Module

Embodiments of the present disclosure may provide an analytics module for making predictions based on an analysis of data accessible to platform 100. The predicative analytics module may be in operative communication with, but not limited to, for example, the patient profile data and the external datasets. The predictive analytics may include, but not be limited to, the following features and functionality.

### Patient Facing: Preventive Care

The predictive analytics module may be configured to track and monitor a plurality of data sources, including, but not limited to profiles stored in a) patient profile database 120, b) external data sets 135, and c) patient tracking data 145. The module may use the data obtained via profile 100 in order to trigger certain actions when the profile data meets the criteria for triggering the actions.

### Patient Profile Data

Consistent with the various embodiments herein, the module may have access to coded records associated with, for example, patient encounters, patient allergies, patient medical history, and the like. In one example, the module may analyze the coded records in a patient profile in order to determine if a profile qualifies for an alert. In other example, the module may analyze the profile data based on, for example, certain characteristics of the profile data. The characteristics may be derived from, for example, the data point entries in the various patient encounter notes within the patient profile data. The process for determining whether the patient profile qualifies for an alert is detailed below.
A) Patient Tracking Data

In yet further embodiments, the predictive analytics module may be in communication with patient monitoring and tracking data, as provided by the patient tracking module 145. The patient tracking data may comprise up-to-date information on, for example, but not limited to, a patient's biometrics, location, destination, and various other telemetric data that may be collected by the patient tracking module 145. Patient tracking module 145 may be configured to monitor and track the data by way of, for example, but not limited to, a computing device associated with the patient and various peripherals thereto.

For example, patient tracking module 145 may be integrated with wearable computing devices configured to track, for example, a patient's heart rate, respiratory rate, coughing, and blood oxygen levels. In this way, patient tracking module 145 may be enabled to receive biometric data associated with a patient. As another example, patient tracking module 145 may be in operative communication with a mobile computing device having a location detection module (e.g., a smartphone). In turn, patient tracking module 145 may associate the tracked and monitored data (i.e., biometrics and location) with the patient profile, thereby supplementing the patient profile data with up-to-date patient data.
B) External Data

Still consistent with the various embodiments herein, the module may receive data from external data sources 135. External data sources 135 may be accessed by the module in order to share data including, for example, weather data, allergen data, and the like. External data sources 135 may also provide information which includes, for example, but is not limited to, Medical Encyclopedias, WHO databases, and up-to-date diagnostics from various other data sources.

### Preventive Care Analysis

Consistent with the embodiments of the present disclosure, the predictive analytics module may be configured to determine and/or suggest a preventive action, a plurality of preventive actions, or a series of suggested preventive actions to be taken by the patient in accordance to a plurality of methods, and through a plurality of systems. Some of those methods and systems include, but are not limited to, for example:
Analysis performed based on a defined set of rules and conditions associated with a plurality of patient data including at least one of: patient profile data, patient tracking data, external data;
   Wherein the plurality of patient data further comprises data elements;
   Wherein the data elements further comprise at least one of: coded records, patient encounter data, patient monitoring data, external databases, external diagnostic data;
Analysis performed on data elements based on the rules and conditions;
   Wherein rules and conditions include a patient data element;
Analysis performed on external data to determine whether an element of the external data meets a predetermined threshold;
   Wherein the predetermined threshold is associated with the set of conditions;
   Wherein the set of conditions may impact one or more patients;
   Wherein a positive association match between the external patient data element meeting the predetermined threshold and a patient data element triggers an alert;
Analysis performed on patient data to determine whether profile qualifies for an alert;
Analysis performed on patient data for a plurality of patients to determine whether one or more patients qualifies to receive an alert;

The predictive analytics module further provides methods and systems for making predictions based on an analysis of data accessible to platform 100. Platform 100 may monitor data sources in real-time such that the information available to platform 100 is consistently updated with the most up-to-date information.

The predictive analytics module may further be configured to correlate patient data against a certain set of rules and conditions. The module may trigger alerts and notifications as a result of analyzing external data relating to the set of conditions and making the determination that certain conditions are present which may impact specific patients.

By way of non-limiting example, the predictive analytics module may be configured to operate using the rules and conditions relating to, for example, an external data element such as a threshold data point exhibiting a high pollen count.

In one embodiment, the predictive analytics module may be configured to analyze this external data element to determine whether or not a threshold data point has been met or exceeded. After such a determination is made, the module may then be configured to determine the set of conditions associated with the threshold point being exceeded. The predictive analysis module may be further configured to determine if any of the patient data elements correspond to the set of conditions associated with the threshold point being met or exceeded. The module further configured to determine implications of said rule or condition on qualifying patient profiles based on patient data elements having a positive match or association with the rule or conditions associated with the threshold point being met or exceeded.

As detailed above, the patient profile data relevant to the qualified rule or condition may be determined by the various codes and/or characteristics within the plurality of patient data. Those profiles that have been qualified may then be determined to be relevant to a triggering action associated with the rule and condition. Triggering actions may include, but not be limited to, for example, alerts and or notifications sent to at least one relevant party or entity related to the profile data.

By way of non-limiting example, the predictive analytics module may be configured to be used by a patient, for example, in waypoint navigation from a first destination to a second destination.

In an embodiment, the predictive analytics module, may be configured to trigger alerts to a practitioner and/or patients to ensure the patient takes preventive measures to avoid aggravating or impacting a condition associated with a predetermined threshold being met or exceeded.

By way of a non-limiting example, let's assume that the module has analyzed data and have determined a trend associated with a patient having a diagnosis of Asthma. In this example, the module may have been configured to determine that there exists a high probability that a patient with said diagnosis will have a negative reaction when exposed to air quality with a value above a predetermined threshold.

Based on analysis of external data received by the module, it has been determined that the air quality has met or exceeded the predetermined threshold. That patient is using a navigation application on their mobile device to navigate to a destination. The predictive analytics module may be configured such that it receives information associated with the patient location associated with the patient's mobile device. The patient's mobile device also includes applications which track patient data including, but not limited to, biometric and health information.

The predictive analytics modules may be configured to determine that based on their current route of travel, there is a high probability that the patient will encounter air quality which they should avoid (i.e., fire in the area). As a result, the predictive analytics module can alert the patient, automatically reroute the patient, or suggest that the patient change their route and avoid the affected area.

The aforementioned analyses may be based on, at least in part, a determination of which data elements or predetermined thresholds frequently occur together within an encounter note and/or within an input tier of an encounter note, and/or within a plurality of patient data. In certain embodiments, the predictive analytics module may work in conjunction with favorites module and/or recommendation module. For instance, the predictive analytics module may be configured in a manner that user input is required at each step. In this configuration, the favorites module 125 may determine which data points or data point categories to display within favorites module associated with data entry for the predictive analytics module. For example, the most frequently occurring suggested actions associated with a particular threshold or data element can be determined by the favorites module.

As noted above, the predictive analytics module may be configured to work in conjunction with the recommendation module. For instance, the predictive analytics module may be configured in a manner that user input is required at each step. In this configuration, the recommendation module may provide recommended actions to a user 105 (i.e., avoid this location, reroute your navigation away from this area, avoid outdoors, or another preventive action) based on determinations made by the predictive analytics module.

In other embodiments, all or a portion of this functionality may exist entirely within the predictive analytics module. In yet some other embodiments, the determination and triggering of alerts and suggested preventive actions for the patient is performed automatically or autonomically without the aid of a user.

### a) Doctor Facing: Preemptive Preparedness

As mentioned above, the predictive analytics module further provides methods and systems for making predictions based on an analysis of data accessible to platform 100. Platform 100 may monitor data sources in real-time such that the information available to platform 100 is consistently updated with the most up-to-date information. The predictive analytics module may be configured to correlate patient data against a certain set of rules and conditions. The module may trigger alerts and notifications as a result of analyzing external data relating to the set of conditions and making the determination that certain conditions are present which may affect specific patients.

By way of non-limiting example, the predictive analytics module may be configured to be used by a practitioner, for example, to predict how many walk-ins the practitioner may have and/or how many patients may need help/advice today on certain topics. This will help the practitioner plan/prepare.

### Preemptive Preparedness Analysis

Consistent with the embodiments of the present disclosure, the predictive analytics module may be configured to determine and/or suggest a preemptive preparedness action, a plurality of preemptive preparedness actions, or a series of suggested preemptive preparedness actions to be taken by the practitioner in accordance to a plurality of methods, and through a plurality of systems. Some of those methods and systems include, but are not limited to, for example:
Analysis performed based on a defined set of rules and conditions associated with a plurality of patient data including at least one of: patient profile data, patient tracking data, external data;
   Wherein the plurality of patient data further comprises data elements;
   Wherein the data elements further comprise at least one of: coded records, patient encounter data, patient monitoring data, external databases, external diagnostic data;
Analysis performed on data elements based on the rules and conditions;
   Wherein rules and conditions include a patient data element;
Analysis performed on external data to determine whether an element of the external data meets a predetermined threshold;
   Wherein the predetermined threshold is associated with the set of conditions;
   Wherein the set of conditions may impact one or more patients;
   Wherein a positive association match between the external patient data element meeting the predetermined threshold and a patient data element triggers a practitioner alert;
Analysis performed to determine a correlation between impacted patients and data elements associated with a practitioner;
   Wherein data elements associated with a practitioner further comprises at least one of: practitioner schedule, practitioner availability, practitioner support staff schedule, practitioner support staff availability, available/stocked supplies, needed supplies, ordered supplies, supplies scheduled for delivery, beds available, beds needed, quarantine rooms available, quarantine rooms needed, or another preparedness action;
   Where data elements associated with impacted patients further comprises at least one of: potentially mild symptoms, potentially moderate symptoms, potentially severe symptoms, or another data element associated with impacted patients.
Analysis performed on the correlation of data to determine a preemptive preparedness action/suggestion/series of actions/suggestions;
   Wherein the preemptive preparedness action further comprises alerting the practitioner to prepare for at least one of: a high number of walk-ins, a moderate number of walk-ins, a low number of walk-ins, an average number of walk-ins, or another preemptive preparedness action;
   Wherein the preemptive preparedness action further comprises informing the practitioner of the occurrence of at least one of: an outbreak, an epidemic, an extreme crisis, a natural disaster, another preemptive preparedness action;
   Wherein the preemptive preparedness action further comprises advising the practitioner to prepare for at least one of the following: a high volume of patient calls, a moderate volume of patient calls, a low volume of patient calls, an average number of patient calls, or another preemptive preparedness action;
   Wherein the preemptive preparedness action further comprises suggesting to the practitioner at least one of the following: schedule more practitioner support staff, schedule more on duty practitioners, maintain level of practitioner support staff, reduce level of practitioner support staff, maintain level of available practitioners, reduce level of available practitioners, order more supplies, stock delivered supplies, increase number of beds available, maintain average number of beds available, increase number of quarantine rooms available, maintain number of quarantine rooms available, or another preemptive preparedness action.
Analysis performed on patient data to determine whether a practitioner qualifies to receive an alert associated with patient;
   Wherein analysis further comprises determining whether the necessary practitioner is at least one or more of: a general practitioner, a specialized practitioner, or other type of practitioner.
Analysis performed on patient data for a plurality of patients to determine whether one or more practitioners qualifies to receive an alert;
Analysis performed on external data to determine whether the probability of the occurrence an event has reached a predetermined threshold;
   Wherein the event impacts patients or a subset of patients;
   Wherein the event is at least one of: high pollen count, poor air quality, high smog level, high lead level in public water supply, poisonous gas released, high radiation levels, terror attack, fire, flooding, earthquake, tornado, heat wave, drought, or another type of emergent event.

As a consequence of the analysis conducted by the predictive analytics module, a positive correlation between a set of conditions associated with data elements within external data and data elements within patient data determines the level of probability of a possible set of occurrences. Once the probability passes a predetermined threshold value, the predictive analytics module may be further configured to correlate the occurrence/event with the patient data to determine how many patients would be impacted. The predictive analytics module may be further configured to trigger alerts to a practitioner as a consequence of analysis of patient data based on this correlation.

This system allows doctors and health practitioners to prepare to provide certain types of care based on the predictive analysis of patient data. By way of non-limiting example, the predictive analytics module may be further configured to be used by a practitioner, for example, to predict how many walk-ins the practitioner may have based on the high probability of an event occurring. For example, when a smog alert is either issued or predicted by the weather forecast, the module will alert the practitioner to prepare to receive a large number of asthma patients or patients with respiratory problems as walk-ins. In another example, a measles outbreak at a local elementary school may cause the module to suggest to the practitioner to take a number of preemptive preparedness actions (prepare a certain number of quarantine rooms, schedule more practitioner support staff, schedule more available practitioners, request more specialized practitioners, or other type of preemptive preparedness action).

In yet another non-limiting example, the public diagnosis of a popular celebrity with a severe health malady may cause the module to alert the practitioner to prepare to receive a high volume of walk-ins, calls and/or emails relating to the severe health malady. In another possible embodiment, in the event of a terror alert, probable terror attack, or forecasted natural disaster, the module may be configured to suggest a series of preemptive preparedness actions.

The aforementioned analyses may be based on, at least in part, a determination of which data elements, events, preemptive preparedness actions, or predetermined thresholds frequently occur together within an encounter note and/or within an input tier of an encounter note, and/or within a plurality of patient data and/or within external data, and/or within associated data.

In certain embodiments, the predictive analytics module may work in conjunction with favorites module and/or recommendation module. For instance, the predictive analytics module may be configured in a manner that user input is required at each step. In this configuration, the favorites module 125 may determine which data points or data point categories to display within favorites module associated with data entry for the predictive analytics module. For example, the most frequently occurring preemptive preparedness actions associated with a particular threshold or data element can be determined by the favorites module.

As noted above, the predictive analytics module may be configured to work in conjunction with the recommendation module. For instance, the predictive analytics module may be configured in a manner that user input is required at each step. In this configuration, the recommendation module may provide recommended preemptive preparedness actions to a user 105 (i.e., high number of patient calls, high number of walk-ins, low level of necessary supplies, low number of practitioner support staff available, or other type of preemptive preparedness action) based on determinations made by the predictive analytics module.

In other embodiments, all or a portion of this functionality may exist entirely within the predictive analytics module. In yet some other embodiments, the determination and triggering of alerts and suggested preventive actions for the patient is performed automatically or autonomically without the aid of a user.

### Clinical Data Integration System

The platform 100 may serve as a clinical data integration system. The clinical data integration system may provide a unified platform for accessing and managing electronic health record data across multiple heterogeneous healthcare information systems. The platform may operate as an intelligent middleware layer positioned between clinical end users and one or more underlying electronic health record systems. This middleware layer may abstract the complexity of disparate backend systems while providing a consistent and adaptive user experience. The platform may enable healthcare providers to access patient information, document clinical encounters, and execute clinical workflows without requiring direct interaction with multiple separate systems.

The clinical data integration system may provide a unified user interface layer that serves as the primary interaction environment for healthcare providers, abstracting away the underlying complexity of multiple heterogeneous electronic health record systems. Rather than requiring providers to directly interact with individual EHR interfaces, the system may present a consistent, platform-agnostic interface layer through which all clinical data access and workflow operations are performed. This unified interface layer may render clinical information from multiple backend EHR systems within a single, coherent presentation framework, enabling providers to work within a stable interaction environment regardless of which underlying systems are providing the data.

The platform-agnostic interface layer may implement a comprehensive abstraction architecture that translates provider interactions into appropriate backend operations across different EHR platforms. When a provider performs actions within the unified interface, such as reviewing patient histories or entering clinical notes, the system may automatically translate these interactions into the specific API calls, user interface automation sequences, or data access operations required by each underlying EHR system. This abstraction enables providers to maintain consistent workflows and interaction patterns while the system handles the technical complexity of interfacing with diverse backend systems.

The unified interface layer may maintain its own presentation logic, data visualization components, and workflow orchestration mechanisms that operate independently of the underlying EHR systems'native interfaces. For example, when displaying patient laboratory results, the unified interface may apply consistent formatting, sorting, and filtering options regardless of whether the data originates from Epic, Cerner, AllScripts, or other EHR platforms. This approach ensures that providers experience a stable, predictable interface environment that does not change based on the source systems providing the underlying clinical data.

The platform may address fundamental challenges in healthcare information technology environments where multiple electronic health record systems coexist within a single healthcare organization or network. Healthcare providers may encounter situations where patient data resides in different systems operated by different departments, facilities, or affiliated organizations. The platform may eliminate the need for providers to learn and navigate multiple distinct user interfaces by presenting a unified interface layer that adapts to the provider's role, current workflow context, and individual preferences. This unified approach may reduce cognitive burden on clinical users and may improve workflow efficiency.

The platform may employ intelligent routing mechanisms to determine optimal pathways for accessing clinical data in real time. When a healthcare provider requests patient information, the platform may evaluate multiple factors to select the most appropriate method for retrieving that information. These factors may include the availability of application programming interfaces, the current health status of various data sources, institutional policy constraints, and the specific type of data being requested. The platform may dynamically switch between different access methods based on changing conditions, thereby maintaining workflow continuity even when primary data sources experience degraded performance or temporary unavailability.

The platform may maintain comprehensive provenance tracking for all clinical data accessed through the system. Each piece of information displayed to a user may be accompanied by metadata indicating the source system from which the data originated, the method used to retrieve the data, the timestamp of retrieval, and indicators of data freshness. This transparency may enable healthcare providers to make informed clinical decisions with full awareness of the currency and reliability of the information they are viewing. The provenance tracking may also support regulatory compliance and audit requirements by maintaining detailed records of all data access and system actions.

The provenance metadata annotation system may optionally implement cryptographic signing mechanisms to ensure immutable attestation of clinical data access operations and system pathway selections. In some embodiments, the system may generate cryptographic signatures for provenance records using digital signing algorithms, enabling verification that pathway selection decisions, performance metrics, and data retrieval operations have not been altered after initial recording. The system may also support anchoring provenance metadata to immutable storage systems, such as distributed ledger technologies, to provide tamper-evident audit trails for regulatory compliance and clinical accountability purposes.

The optional immutability features may enable healthcare institutions to maintain verifiable records of all clinical data access operations, pathway performance metrics, and system failover events. When cryptographic attestation is enabled, the system may generate hash-based integrity proofs for each data access transaction, pathway selection decision, and performance measurement, creating an immutable chain of evidence that can be independently verified by auditors, regulatory bodies, or legal proceedings. This capability may be particularly valuable in clinical research environments, regulatory compliance scenarios, or medicolegal contexts where the integrity and authenticity of clinical data access records must be demonstrably preserved.

The digital signing algorithms may be implemented using hardware security modules (HSMs) or trusted platform modules (TPMs) to ensure that cryptographic keys remain protected from unauthorized access or tampering. The system may generate cryptographic signatures using elliptic curve digital signature algorithms that provide equivalent security to RSA signatures while requiring smaller key sizes and faster computation times. For high-throughput clinical environments, the system may implement batch signing mechanisms that aggregate multiple provenance records into single cryptographic operations to optimize performance while maintaining individual record integrity.

The immutable attestation capabilities may support multiple levels of cryptographic assurance based on the sensitivity and importance of different clinical data operations. For standard data access operations, the system may generate simple hash-based integrity checksums that enable detection of accidental modifications. For critical patient safety events or regulatory compliance scenarios, the system may implement full digital signatures with timestamping services that provide non-repudiation guarantees and precise temporal attestation of when each operation occurred.

The provenance metadata structure may be designed to accommodate cryptographic elements without disrupting existing clinical workflows or data processing operations. The system may embed cryptographic signatures and hash values as optional metadata fields that can be ignored by systems that do not require cryptographic verification, ensuring backward compatibility with existing clinical information systems. This approach enables healthcare institutions to gradually adopt cryptographic attestation capabilities without requiring simultaneous upgrades to all connected systems and applications.

The cryptographic signing mechanisms may implement industry-standard digital signature algorithms such as (but not limited to) RSA-2048, ECDSA with P-256 curves, or EdDSA using Ed25519 keys to generate tamper-evident signatures for provenance metadata records. When cryptographic attestation is enabled, the system may generate a unique digital signature for each data access operation by creating a hash digest of the complete provenance record and signing it with a private key maintained in a hardware security module or secure key management system. The resulting digital signatures may be embedded within the provenance metadata structure, enabling subsequent verification using the corresponding public key to confirm that pathway selection decisions, performance metrics, and data retrieval timestamps have not been modified after initial recording.

The hash-based integrity proofs may implement cryptographic hash functions such as (as non-limiting examples) SHA-256 or SHA-3 to create unique fingerprints for each data access transaction and system operation. The system may generate merkle tree structures that organize multiple hash values into hierarchical proof systems, enabling efficient verification of large sets of provenance records without requiring access to the complete underlying data. For example, when a clinical session involves multiple data panel operations, the system may create individual hash values for each panel's provenance record, then combine these hashes into a merkle root that provides a single cryptographic proof for the entire session's integrity.

The immutable storage anchoring capabilities may interface with distributed ledger technologies, blockchain networks, or other tamper-evident storage systems to create permanent records of critical provenance metadata. The system may periodically batch provenance records and submit cryptographic hash summaries to immutable storage networks, creating timestamped attestations that can be independently verified by external parties. In some embodiments, the system may implement smart contract interfaces that automatically record provenance hash values to blockchain networks at predetermined intervals, such as every hour or at the completion of each clinical session.

The verification mechanisms may enable healthcare institutions, auditors, or regulatory bodies to independently validate the integrity of clinical data access records using standard cryptographic verification tools. The system may provide verification APIs that accept provenance records and their associated digital signatures, then perform cryptographic validation to confirm authenticity and detect any unauthorized modifications. For regulatory compliance scenarios, the system may generate compliance reports that include cryptographic proofs demonstrating the integrity of all data access operations during specified time periods.

The optional immutability features may be configured based on institutional policies, regulatory requirements, or specific clinical use cases. For routine clinical operations, the system may operate with standard provenance logging without cryptographic attestation to optimize performance. However, for clinical research studies, regulatory audits, or medicolegal proceedings, administrators may enable full cryptographic signing and immutable storage anchoring to provide the highest level of data integrity assurance. The system may maintain separate cryptographic key pairs for different types of operations, enabling fine-grained control over which data access operations receive cryptographic attestation.

The cryptographic attestation system may implement key rotation mechanisms that periodically update signing keys while maintaining backward compatibility for verification of historical records. The system may maintain a key management database that tracks the validity periods for different cryptographic keys, enabling proper verification of provenance records created with previously valid keys. This approach ensures that historical clinical data access records remain verifiable even after cryptographic keys have been rotated for security purposes.

The platform may provide adaptive user interface capabilities that respond to the context of each user session. The interface may adjust its layout, visible components, and available actions based on the authenticated user's role within the healthcare organization, the current phase of the clinical workflow, and the specific task being performed. Healthcare providers may save customized workspace configurations as context snapshots that can be restored in future sessions, enabling consistent working environments across different login sessions and different underlying electronic health record systems. This adaptability may support diverse clinical workflows while maintaining a familiar and efficient user experience for each individual provider.

The platform may employ natural language processing capabilities to interpret user commands and queries while maintaining strict safety constraints. When a user provides input in natural language form, the system may parse the input to identify the underlying intent. The system may maintain a whitelist of approved intents that correspond to specific system operations. Each whitelisted intent may represent a predefined action that the system is authorized to perform. The natural language processing module may compare the parsed input against the whitelist to determine whether the input corresponds to an approved intent. If the input does not match any whitelisted intent, the system may reject the input and may prompt the user to rephrase or clarify their request.

The whitelisted intent framework may prevent unpredictable system behavior by constraining natural language interactions to a finite set of known operations. Each whitelisted intent may be associated with specific parameters that define the scope and constraints of the corresponding action. The system may extract parameter values from the natural language input to populate the intent structure. For example, a natural language input such as “show recent lab results” may be mapped to a whitelisted intent for retrieving laboratory data, with parameters specifying the time range and data type. The system may validate extracted parameters against predefined rules to ensure they fall within acceptable ranges.

The intent processing module may decompose complex intents into sequences of primitive operations. Each primitive operation may correspond to a single data retrieval action or system command. The system may execute these primitive operations in a defined order to fulfill the user's request. The decomposition process may enable the system to handle sophisticated queries while maintaining control over each individual operation. The system may validate each primitive operation before execution to ensure it complies with security policies and user permissions.

The natural language interface may support disambiguation when user input could map to multiple possible intents. The system may present disambiguation options to the user when the input is ambiguous. The user may select the intended meaning from the presented options. Once the user clarifies their intent, the system may proceed with the corresponding whitelisted intent. This disambiguation process may ensure that the system executes only the action that the user actually intended.

The system may maintain an audit trail of all natural language inputs and their corresponding intent mappings. Each natural language interaction may be logged with the original input text, the identified intent, the extracted parameters, and the resulting system actions. This audit trail may provide transparency into how natural language commands are interpreted and executed. The audit records may support compliance requirements and may enable administrators to review system behavior.

The routing engine may serve as the central decision-making component for determining how to access clinical data. When the system receives a data access request, the routing engine may evaluate multiple factors to select the optimal access pathway. The routing engine may first determine whether an application programming interface is available for the requested data type. The system may maintain a capability registry that catalogs which data types and operations are accessible via application programming interfaces for each connected electronic health record system. The routing engine may query this capability registry to determine API availability.

The routing engine may also evaluate the current health status of available access pathways. The health monitoring module may continuously track performance metrics for each pathway, including response times, error rates, and availability status. The routing engine may retrieve these health metrics in real time when making routing decisions. If the application programming interface pathway is experiencing degraded performance, the routing engine may select an alternative pathway even if the API is technically available. The routing engine may apply threshold rules to determine when performance degradation warrants switching to an alternative pathway.

The routing engine may consider institutional policy constraints when selecting an access pathway. Certain types of data or operations may be subject to policy rules that mandate or prohibit specific access methods. For example, institutional policy may require that medication orders be submitted only through application programming interfaces to ensure proper validation and safety checks. The routing engine may enforce these policy constraints by filtering available pathways based on the requested operation type. The system may maintain a policy database that defines these constraints for different data types and operations.

The routing engine may implement a scoring mechanism to compare available pathways. Each pathway may receive a score based on multiple factors including availability, current health status, historical reliability, and policy preferences. The routing engine may calculate scores for all available pathways and may select the pathway with the highest score. The scoring algorithm may weigh different factors according to configurable priorities. For example, the system may prioritize API-based access over automation-based access when both are available and healthy.

The routing engine may support dynamic pathway switching during request execution. If the initially selected pathway fails or experiences errors during execution, the routing engine may automatically redirect the request to an alternative pathway. This failover capability may ensure continuity of data access even when individual pathways experience transient failures. The system may log all pathway switches in the audit trail to maintain visibility into access patterns.

The health monitoring module may continuously assess the operational status of all system components and data sources. The module may collect performance metrics from multiple sources including application programming interface connectors, automation engines, and data normalization services. The health monitoring module may aggregate these metrics to provide a comprehensive view of system health. The module may track response times for each type of data access operation. Response time measurements may include the time required to establish connections, transmit requests, process data, and receive responses.

The health monitoring module may calculate error rates for each access pathway. Error rates may be computed as the ratio of failed requests to total requests over a defined time window. The module may track different types of errors separately, including timeout errors, authentication failures, data validation errors, and system unavailability errors. The module may maintain separate error rate metrics for different data types and operations to enable fine-grained health assessment.

The health monitoring module may implement threshold-based alerting. The module may compare current metrics against predefined threshold values to determine when system health has degraded below acceptable levels. When a metric exceeds its threshold, the module may generate an alert notification. Alert notifications may be transmitted to system administrators and may also trigger automated responses such as pathway switching or failover activation. The module may support configurable threshold values to accommodate different performance requirements for different data types.

The health monitoring module may maintain historical performance data to enable trend analysis. The module may store time-series data for key metrics including response times, error rates, and availability percentages. This historical data may be analyzed to identify patterns and trends in system performance. The module may detect gradual performance degradation that might not trigger immediate threshold alerts but could indicate developing issues. The module may generate predictive alerts based on trend analysis to enable proactive intervention before performance degrades to unacceptable levels.

The health monitoring module may provide a health status dashboard for system administrators. The dashboard may display real-time metrics for all monitored components and pathways. The dashboard may use visual indicators such as color coding to highlight components with degraded health status. The dashboard may enable administrators to drill down into detailed metrics for specific components or time periods. The dashboard may also display historical trends and may provide tools for analyzing performance patterns.

The data normalization module may transform clinical data from heterogeneous source formats into a unified presentation format. When clinical data is retrieved from an underlying electronic health record system, the data may be in a format specific to that system. The data normalization module may receive this source-specific data and may apply transformation rules to convert it to a standardized format. The standardized format may be designed to support consistent presentation across all data sources regardless of their native formats.

The data normalization module may perform schema mapping to align source data structures with the unified schema. Each source system may organize data using different field names, data types, and hierarchical structures. The normalization module may maintain mapping definitions that specify how each source field corresponds to a field in the unified schema. The module may apply these mappings to restructure source data into the unified format. The mapping process may handle differences in data granularity, such as when a single source field maps to multiple unified fields or when multiple source fields must be combined into a single unified field.

The data normalization module may perform terminology translation to standardize clinical codes and terms. Different electronic health record systems may use different coding systems for diagnoses, procedures, medications, and laboratory tests. The normalization module may translate source codes to standardized terminology systems such as SNOMED CT, LOINC, or RxNorm. The module may maintain terminology mapping tables that define equivalences between different coding systems. When a source code does not have a direct equivalent in the target terminology system, the module may map to the closest available concept or may preserve the original code with an indicator that no standard mapping is available.

The data normalization module may perform unit conversion to standardize measurements. Clinical measurements may be expressed in different units depending on the source system and regional conventions. The normalization module may convert measurements to standard units to enable consistent presentation and comparison. For example, laboratory values may be converted from conventional units to SI units or vice versa. The module may maintain conversion factors for different measurement types and may apply appropriate conversions based on the data type and source units.

The data normalization module may validate normalized data to ensure integrity and completeness. The module may check that required fields are present and contain valid values. The module may verify that data types are correct and that values fall within expected ranges. The module may detect and flag anomalous values that may indicate data quality issues. When validation errors are detected, the module may log the errors and may either reject the data or may flag it for review depending on the severity of the issue.

The data normalization module may attach provenance metadata to normalized data. The metadata may include the identifier of the source system, the access method used to retrieve the data, the timestamp of retrieval, and indicators of data freshness. This provenance information may be preserved throughout the data lifecycle and may be displayed to users to provide transparency about data origins. The provenance metadata may enable users to assess the reliability and currency of displayed information.

The intent processing module may provide a controlled mechanism for interpreting user commands expressed in natural language. The module may receive natural language input from the user interface layer. The input may be in the form of typed text or transcribed speech. The intent processing module may parse the input to extract semantic meaning. The parsing process may involve tokenization, part-of-speech tagging, and syntactic analysis. The module may identify key entities and relationships within the input text.

The intent processing module may classify the parsed input to determine the user's intended action. The classification process may compare the input against patterns associated with whitelisted intents. Each whitelisted intent may be defined by a set of linguistic patterns, keywords, and structural characteristics. The module may use pattern matching algorithms or machine learning classifiers to identify the best matching intent. The classification process may generate a confidence score indicating the likelihood that the identified intent is correct.

The intent processing module may extract parameters from the natural language input. Parameters may include patient identifiers, date ranges, data types, and other values that specify the scope of the requested action. The module may use named entity recognition techniques to identify parameter values within the input text. The module may validate extracted parameters to ensure they conform to expected formats and constraints. When required parameters are missing from the input, the module may prompt the user to provide the missing information.

The intent processing module may handle ambiguous inputs by presenting disambiguation options to the user. When the input could map to multiple whitelisted intents with similar confidence scores, the module may generate a list of possible interpretations. The module may present these interpretations to the user and may request clarification. The user may select the correct interpretation from the presented options. Once the user provides clarification, the module may proceed with the selected intent.

The intent processing module may reject inputs that do not correspond to any whitelisted intent. When the classification process fails to identify a matching intent with sufficient confidence, the module may inform the user that the request cannot be processed. The module may provide guidance to help the user rephrase their request in a form that can be mapped to a whitelisted intent. This rejection mechanism may prevent the system from attempting to execute unrecognized or potentially unsafe operations.

The intent processing module may convert validated intents into structured request objects. Each request object may contain the intent type, extracted parameters, and metadata about the original input. The module may transmit these request objects to the routing engine for execution. The structured format may enable consistent processing of requests regardless of how they were originally expressed. The module may log all intent processing activities including the original input, identified intent, extracted parameters, and any errors or rejections.

The provider profile module may maintain user-specific preferences and configurations that persist across different electronic health record systems. Each healthcare provider may have a profile that is linked to their provider identity rather than to any specific electronic health record system. The profile may include layout preferences that define how user interface elements are arranged on the screen. The profile may specify which panels are visible by default and how they are positioned relative to each other. The profile may include filter settings that define default criteria for displaying data such as date ranges or data categories.

The provider profile module may maintain a global interaction model that transcends individual EHR system boundaries, ensuring that healthcare providers experience consistent behavioral patterns and workflow sequences regardless of which backend systems are active. This global interaction model may encompass not only visual presentation preferences but also fundamental interaction paradigms, such as navigation patterns, data entry sequences, and clinical decision support workflows. Unlike conventional EHR user settings that are confined to individual systems, the global provider experience may persist across all connected healthcare platforms, creating a stable interaction environment that remains constant even when providers move between different facilities or access different EHR systems.

In some embodiments, the provider-specific interaction profile defines a consistent interaction model that is presented through a unified user interface layer, such that core workflows and interactions remain stable even when underlying electronic health record systems differ in native interface design.

The stable interaction model may implement consistent behavioral responses to provider actions across all backend EHR systems. For example, when a provider performs a specific gesture or keyboard shortcut to access patient allergies, the system may ensure that this action produces the same result and follows the same interaction sequence regardless of whether the underlying data is sourced from Epic's allergy module, Cerner's clinical documentation system, or a standalone allergy management application. This behavioral consistency may extend beyond simple preference settings to encompass complex workflow patterns, clinical decision trees, and multi-step data entry processes.

The system may distinguish between superficial visual customization and fundamental interaction model consistency by maintaining separate layers for presentation styling and behavioral logic. While the visual appearance of clinical data may adapt to accommodate different screen sizes, device capabilities, or institutional branding requirements, the underlying interaction model may remain constant across all environments. This approach ensures that providers can rely on muscle memory, established workflow patterns, and learned interaction sequences regardless of the specific EHR platforms or healthcare facilities they are working within.

The provider profile module may store workflow configurations that define sequences of actions or views that the provider commonly uses. A workflow configuration may specify a series of screens or data views that the provider typically accesses in a particular order. The module may enable the provider to activate a saved workflow configuration to quickly navigate through a predefined sequence of views. This capability may reduce the time required to access commonly needed information.

The provider profile module may maintain a history of the provider's interactions with the system. The interaction history may include records of frequently accessed data types, commonly used commands, and typical workflow patterns. The module may analyze this interaction history to identify patterns and preferences. The module may use these identified patterns to provide personalized suggestions or to automatically configure interface elements based on the provider's typical usage patterns.

The provider profile module may map a single provider identity to multiple local user accounts across different electronic health record systems. A healthcare provider may have separate user accounts in different systems operated by different facilities or departments. The provider profile module may maintain associations between the provider's global identity and their local identities in each system. When the provider accesses any of these systems through the platform, the module may apply the provider's global profile consistently regardless of which local account is being used.

The provider profile module may synchronize profile data across multiple devices and sessions. When a provider updates their preferences or saves a new configuration, the module may propagate these changes to all active sessions and may store them persistently for future sessions. This synchronization may ensure that the provider experiences consistent behavior regardless of which device or workstation they use to access the system.

The provider profile module may support profile versioning to enable recovery of previous configurations. When a provider makes changes to their profile, the module may save the previous version before applying the changes. The provider may revert to a previous profile version if they are not satisfied with recent changes. The module may maintain a history of profile versions with timestamps indicating when each version was created.

The provider profile module may enable administrators to define default profiles for different user roles. A default profile may specify standard configurations that are appropriate for a particular role such as physician, nurse, or pharmacist. When a new user account is created, the module may initialize the user's profile with the default settings for their role. The user may subsequently customize their profile to suit their individual preferences.

The system may provide mechanisms for capturing and restoring complete workspace contexts. A context snapshot may represent the complete state of a user's workspace at a particular moment in time. The snapshot may include the current patient context, active panels and views, applied filters, scroll positions, and any other state information that defines the workspace configuration. The system may enable users to save context snapshots on demand by issuing a save command through the user interface.

When a user requests to save a context snapshot, the system may serialize the current workspace state into a persistent format. The serialization process may capture all relevant state information in a structured format that can be stored and later reconstructed. The system may store the serialized snapshot in a context database associated with the user's profile. Each snapshot may be tagged with a user-provided name or description to facilitate later identification and retrieval.

The system may enable users to restore previously saved context snapshots. When a user requests to restore a snapshot, the system may retrieve the serialized state from the context database. The system may deserialize the state information and may apply it to the current session. The restoration process may reconfigure the user interface to match the saved state, including reopening panels, applying filters, and positioning interface elements. The system may reload any data that was visible in the saved context to recreate the workspace as it existed when the snapshot was captured.

Context snapshots may be particularly valuable when users need to switch between different tasks or patients and later return to a previous context. A user may save a snapshot before switching to handle an urgent matter. After addressing the urgent matter, the user may restore the snapshot to immediately return to their previous workspace configuration without manually reconstructing the context. This capability may reduce the time and effort required to resume interrupted workflows.

The system may automatically capture context snapshots at certain trigger points. For example, the system may automatically save a snapshot when a user switches to a different patient or when a user logs out. These automatic snapshots may serve as recovery points that enable users to restore their workspace if their session is interrupted unexpectedly. The system may maintain a limited number of automatic snapshots per user to manage storage requirements.

The system may enable users to share context snapshots with other users. A user may save a snapshot and may designate it as shareable. Other users with appropriate permissions may access and restore the shared snapshot. This sharing capability may facilitate collaboration and may enable experienced users to share optimized workspace configurations with colleagues. Shared snapshots may be particularly useful for training purposes or for standardizing workflows across a team.

The system may implement resilience mechanisms to maintain workflow continuity when primary data sources experience failures or degraded performance. The resilience manager component may monitor the health status of all data sources continuously. When the health monitoring module detects that a primary data source has failed or is experiencing unacceptable performance degradation, the resilience manager may be notified. The resilience manager may evaluate alternative sources that could provide the requested data.

Alternative sources may include cached data that was previously retrieved from the primary source. The system may maintain a cache of frequently accessed data with configurable expiration policies. When the primary source is unavailable, the resilience manager may direct requests to the cache instead. The system may clearly label cached data with freshness indicators that show when the data was last updated. These freshness indicators may enable users to assess whether the cached data is sufficiently current for their clinical decision-making needs.

Alternative sources may also include secondary application programming interfaces or read replicas of the primary data source. Some electronic health record systems may provide multiple access points for redundancy. The resilience manager may maintain a registry of alternative sources for each primary source. When a primary source fails, the resilience manager may consult this registry to identify available alternatives. The resilience manager may select the alternative source that provides the best combination of data freshness, availability, and performance.

The resilience manager may implement automatic failover when primary sources become unavailable. When a failure is detected, the resilience manager may immediately redirect pending and future requests to an alternative source without requiring user intervention. This automatic failover may minimize workflow disruption by maintaining data access even during system outages. The system may display notifications to users indicating that data is being served from an alternative source and explaining any limitations or freshness constraints.

The resilience manager may continuously monitor primary sources for recovery. When a failed primary source returns to healthy operation, the resilience manager may detect the recovery through ongoing health checks. The resilience manager may orchestrate a return to normal operation by gradually redirecting traffic back to the recovered primary source. The transition back to the primary source may be performed in a controlled manner to avoid overwhelming the recovering system.

The resilience manager may implement circuit breaker patterns to prevent cascading failures. When a data source experiences repeated failures, the resilience manager may temporarily stop attempting to access that source. This circuit breaker behavior may prevent the system from wasting resources on requests that are likely to fail. After a configured timeout period, the resilience manager may attempt to access the source again to determine whether it has recovered. If the source remains unavailable, the circuit breaker may remain open for an extended period.

The system may provide comprehensive audit logging of all resilience actions. When the resilience manager switches to an alternative source, the system may log the event with details including the failed primary source, the selected alternative source, the reason for the switch, and the timestamp. When the system returns to normal operation, this transition may also be logged. These audit records may provide visibility into system behavior during outages and may support post-incident analysis.

The system may generate structured clinical documentation by aggregating data from multiple sources. The documentation generator component may receive a request to create a clinical note or summary. The request may specify the type of document to be created, such as a progress note, discharge summary, or consultation report. The documentation generator may select an appropriate template based on the document type. The template may define the structure and required sections of the document.

The documentation generator may identify the data elements needed to populate the template. For each required data element, the generator may determine which source systems contain the relevant data. The generator may issue data retrieval requests through the routing engine to obtain the necessary information. The routing engine may select appropriate access pathways for each request based on current system conditions. The retrieved data may be normalized by the data normalization module before being incorporated into the document.

The documentation generator may apply clinical logic to synthesize information from multiple sources. For example, the generator may identify trends in laboratory values by comparing results over time. The generator may highlight abnormal findings by comparing values against reference ranges. The generator may identify medication changes by comparing current medications against previous medication lists. These synthesized insights may be incorporated into the generated document to provide clinical context.

The documentation generator may populate the document template with retrieved and synthesized data. Each section of the template may be filled with relevant information from the appropriate sources. The generator may format the data according to the template specifications, which may include text formatting, tables, or structured data elements. The generator may preserve provenance metadata for each data element included in the document, indicating the source system and retrieval timestamp.

The documentation generator may present the draft document to the user for review and editing. The user may modify any auto-populated content, may add additional narrative text, and may remove irrelevant information. The user interface may clearly distinguish between auto-populated content and user-entered content. The user may approve the final document when they are satisfied with its completeness and accuracy.

The documentation generator may write the completed document back to the appropriate electronic health record system. The write-back operation may be performed through an application programming interface if available. The generator may format the document according to the target system's requirements. The generator may include all provenance metadata in the written document to maintain a complete record of data sources. The target system may return a confirmation with a document identifier that can be used for future reference.

The documentation generator may maintain an audit trail of all document generation activities. The audit trail may include records of which data sources were accessed, what data was retrieved, what modifications the user made, and when the document was finalized and written back. This audit trail may support compliance requirements and may enable review of documentation practices.

The system may implement role-based access controls to ensure that users can only access data and perform actions appropriate to their role. The authorization service may maintain a database of user roles and associated permissions. Each user account may be assigned one or more roles such as physician, nurse, pharmacist, or administrator. Each role may be associated with a set of permissions that define which data types the role can access and which operations the role can perform.

When a user attempts to access data or perform an action, the authorization service may verify that the user's role includes the necessary permissions. The service may check permissions before allowing the routing engine to execute data access requests. If the user lacks the required permissions, the authorization service may deny the request and may return an error message to the user. The system may log all authorization decisions including both granted and denied requests.

The authorization service may support fine-grained permissions that control access at the level of individual data elements or operations. For example, permissions may specify that a particular role can view laboratory results but cannot modify them. Permissions may restrict access to sensitive data categories such as psychiatric notes or substance abuse treatment records. The authorization service may enforce these fine-grained permissions consistently across all access pathways.

The authorization service may integrate with enterprise identity management systems. The service may retrieve user role assignments from centralized directory services such as Active Directory or LDAP. This integration may ensure that role assignments remain synchronized with enterprise-wide user management processes. When a user's role changes in the enterprise directory, the authorization service may automatically update the user's permissions in the platform.

The authorization service may support temporary privilege elevation for specific scenarios. In emergency situations, a user may need to access data that would normally be restricted for their role. The authorization service may provide a mechanism for users to request temporary access with appropriate justification. The request may require approval from an authorized administrator. All temporary privilege elevations may be logged with detailed audit records including the justification and approval.

The system may implement comprehensive security measures to protect patient data and system integrity. All data transmitted between system components may be encrypted using industry-standard encryption protocols. The system may use Transport Layer Security for all network communications. Data stored in databases and caches may be encrypted at rest using strong encryption algorithms. Encryption keys may be managed through a secure key management system with appropriate access controls and rotation policies.

The system may implement authentication mechanisms to verify user identities. Users may be required to provide credentials such as usernames and passwords to access the system. The system may support multi-factor authentication requiring users to provide additional verification such as one-time codes from authentication applications. The system may integrate with enterprise single sign-on systems to enable users to access the platform using their existing enterprise credentials.

The system may implement session management to control user access over time. When a user successfully authenticates, the system may create a session and may issue a session token to the user's client device. The session token may be used to authenticate subsequent requests without requiring the user to re-enter credentials. Sessions may have configurable timeout periods after which users must re-authenticate. The system may automatically terminate sessions after periods of inactivity to reduce the risk of unauthorized access from unattended workstations.

The system may implement network security controls to protect against unauthorized access and attacks. Firewalls may restrict network traffic to only authorized communication paths. The system may implement rate limiting to prevent denial-of-service attacks. The system may monitor for suspicious access patterns and may automatically block or throttle requests that appear to be malicious. All security events may be logged and may be analyzed by security monitoring systems.

The system may undergo regular security assessments and penetration testing to identify and address vulnerabilities. Security patches and updates may be applied promptly to address known vulnerabilities in system components and dependencies. The system may implement secure software development practices including code reviews and security testing as part of the development process.

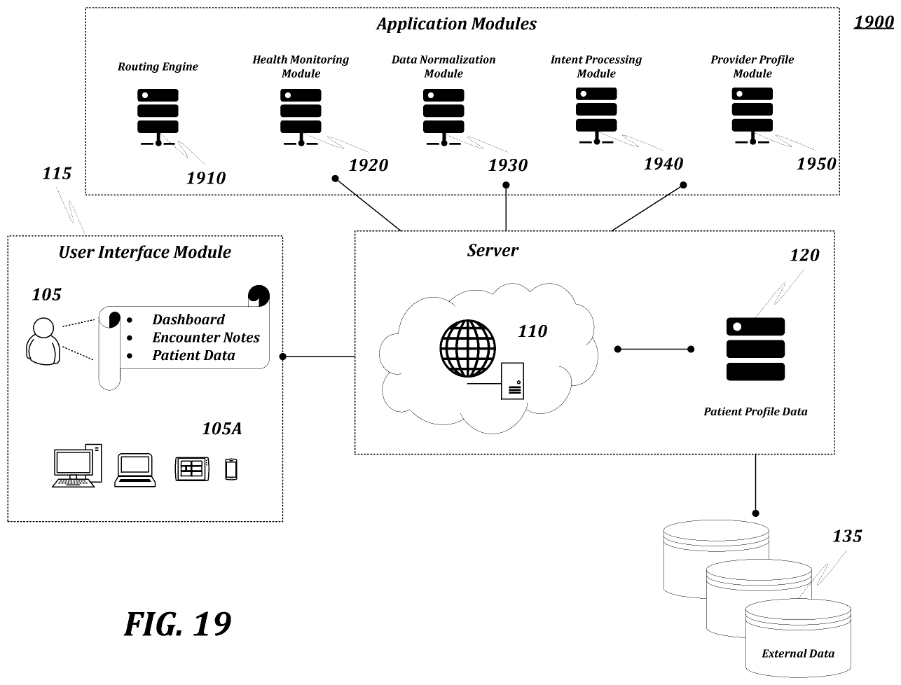

Referring now to FIG. 19, embodiments of the present disclosure provide a software and hardware platform comprised of a distributed set of computing elements, including, but not limited to:

### A. A Routing Engine

A clinical data integration system 1900 may include a routing engine 1910. The routing engine 1910 may incorporate hardware and/or software configured dynamically select between application programming interface (API) and user interface automation pathways for clinical data access requests based on real-time health metrics including response times and error rates, helping to ensure optimal or improved data retrieval performance across heterogeneous electronic health record systems. The routing engine 1910 may receive data access requests from various components of the clinical data integration system 1900. The routing engine 1910 may determine the availability of one or more (e.g., multiple) access pathways for fulfilling each received data access request. The routing engine 1910 may evaluate application programming interface pathways as potential routes for data retrieval operations. The routing engine 1910 may evaluate user interface automation pathways as alternative routes for data retrieval operations. The routing engine 1910 may query a capability registry to determine which access methods are supported for specific data types and operations.

The routing engine 1910 may receive real-time health metrics from a health monitoring module. The routing engine 1910 may analyze response time measurements for each available access pathway. The routing engine 1910 may analyze error rate statistics for each available access pathway. The routing engine 1910 may compare current performance metrics against predefined threshold values. The routing engine 1910 may calculate pathway scores based on multiple performance factors. The routing engine 1910 may weigh different performance factors according to configurable priority settings.

The routing engine 1910 may apply institutional policy constraints when evaluating access pathways. The routing engine 1910 may enforce rules that mandate specific access methods for certain data types. The routing engine 1910 may enforce rules that prohibit specific access methods for certain operations. The routing engine 1910 may consult a policy database that defines constraints for different data categories. The routing engine 1910 may filter available pathways based on the type of operation being requested. The routing engine 1910 may filter available pathways based on the sensitivity level of the requested data.

The routing engine 1910 may select an optimal access pathway from among the available options. The routing engine 1910 may prioritize application programming interface pathways when both application programming interface and automation pathways are available and healthy. The routing engine 1910 may select automation pathways when application programming interface pathways are unavailable or experiencing degraded performance. The routing engine 1910 may select cached data sources when both primary pathways are unavailable. The routing engine 1910 may generate routing decisions that specify which pathway should be used for each data access request.

The routing engine 1910 may transmit routing decisions to appropriate integration components. The routing engine 1910 may direct requests to an application programming interface connector framework when an application programming interface pathway is selected. The routing engine 1910 may direct requests to an automation engine when an automation pathway is selected. The routing engine 1910 may direct requests to a caching subsystem when cached data is selected as the source. The routing engine 1910 may include pathway selection rationale in transmitted routing decisions.

The routing engine 1910 may implement dynamic pathway switching during request execution. The routing engine 1910 may monitor the execution status of routed requests. The routing engine 1910 may detect failures or errors that occur during request processing. The routing engine 1910 may automatically redirect failed requests to alternative pathways without user intervention. The routing engine 1910 may maintain continuity of data access even when individual pathways experience transient failures.

The routing engine 1910 may log all routing decisions in an audit trail. The routing engine 1910 may record the selected pathway for each data access request. The routing engine 1910 may record the factors that influenced each routing decision. The routing engine 1910 may record timestamps associated with routing decisions. The routing engine 1910 may record any pathway switches that occur during request execution. The routing engine 1910 may generate audit records that provide visibility into access patterns and system behavior.

The routing engine 1910 may maintain a routing table that tracks current pathway availability. The routing engine 1910 may update the routing table in response to health status changes. The routing engine 1910 may mark pathways as unavailable when failures are detected. The routing engine 1910 may mark pathways as available when recovery is detected. The routing engine 1910 may maintain separate availability status for different data types and operations.

The routing engine 1910 may implement circuit breaker patterns to prevent cascading failures. The routing engine 1910 may temporarily stop attempting to access pathways that experience repeated failures. The routing engine 1910 may open circuit breakers after a configured number of consecutive failures. The routing engine 1910 may periodically attempt to access failed pathways to detect recovery. The routing engine 1910 may close circuit breakers when successful responses are received from previously failed pathways.

The routing engine 1910 may support configurable routing strategies. The routing engine 1910 may allow administrators to define priority orders for pathway selection. The routing engine 1910 may allow administrators to configure threshold values for performance metrics. The routing engine 1910 may allow administrators to define policy rules that govern pathway selection. The routing engine 1910 may apply configured strategies when making routing decisions.

The routing engine 1910 may provide routing statistics for system monitoring. The routing engine 1910 may track the number of requests routed to each pathway. The routing engine 1910 may track the success rates for each pathway. The routing engine 1910 may track the average response times for each pathway. The routing engine 1910 may transmit routing statistics to a health monitoring dashboard. The routing engine 1910 may enable administrators to analyze routing patterns and system performance.

The routing engine 1910 may coordinate with a resilience manager during failover scenarios. The routing engine 1910 may receive failover notifications when primary sources become unavailable. The routing engine 1910 may receive alternative source recommendations from the resilience manager. The routing engine 1910 may update routing decisions to reflect failover conditions. The routing engine 1910 may coordinate the return to normal operation when primary sources recover.

The routing engine 1910 may evaluate data freshness requirements when selecting pathways. The routing engine 1910 may determine whether cached data is sufficiently current for the requested operation. The routing engine 1910 may compare the age of cached data against freshness thresholds. The routing engine 1910 may select live data sources when freshness requirements cannot be met by cached data. The routing engine 1910 may include freshness considerations in pathway scoring calculations.

The routing engine 1910 may support load balancing across multiple instances of the same pathway type. The routing engine 1910 may distribute requests across multiple application programming interface endpoints when available. The routing engine 1910 may distribute requests across multiple automation workers when available. The routing engine 1910 may apply load balancing algorithms to prevent overloading individual pathway instances. The routing engine 1910 may monitor the load on each pathway instance and adjust distribution accordingly.

The routing engine 1910 may implement request queuing for rate-limited pathways. The routing engine 1910 may queue requests when pathway capacity is temporarily exceeded. The routing engine 1910 may process queued requests when pathway capacity becomes available. The routing engine 1910 may apply timeout policies to queued requests. The routing engine 1910 may redirect requests to alternative pathways if queue wait times exceed acceptable thresholds.

The routing engine 1910 may provide feedback to requesting components about routing outcomes. The routing engine 1910 may indicate which pathway was selected for each request. The routing engine 1910 may indicate whether the request was fulfilled successfully. The routing engine 1910 may indicate whether any pathway switches occurred during request processing. The routing engine 1910 may provide error information when requests cannot be fulfilled by any available pathway.

The routing engine 1910 may implement granular failover mechanisms that operate independently for different data panels and interface components within the unified clinical workspace. Rather than implementing system-wide failover that affects all data access operations simultaneously, the system may maintain separate pathway monitoring and switching logic for individual data views, clinical panels, and workflow components. For example, when the laboratory results panel experiences API connectivity issues with the laboratory information system, the routing engine 1910 may automatically switch that specific panel to user interface automation or cached data sources while maintaining API connectivity for other panels such as medication lists, vital signs, or imaging results.

The granular resilience architecture may enable simultaneous operation of multiple data access pathways within a single clinical workspace session. A provider may simultaneously view patient demographics sourced via API from the primary EHR system, laboratory results retrieved through user interface automation from a secondary laboratory system, and medication information accessed from cached data sources due to temporary pharmacy system maintenance. Each data panel may display appropriate freshness indicators and source attribution while maintaining seamless integration within the unified interface environment.

The per-panel failover system may implement independent health monitoring and pathway selection logic for different types of clinical data and workflow operations. The system may maintain separate performance thresholds, retry logic, and failover criteria for different data categories based on their clinical importance and time sensitivity requirements. For instance, critical patient safety alerts may have more aggressive failover thresholds and faster pathway switching compared to historical clinical notes or administrative data panels. This granular approach ensures that high-priority clinical information remains accessible even when less critical data sources experience performance issues.

### B. A Health Monitoring Module

A clinical data integration system 1900 may include a health monitoring module 1920. The health monitoring module 1920 may incorporate hardware and/or software configured to monitor response times and error rates for both API and user interface automation pathways, generating real-time health metrics that enable the routing engine to make informed pathway selection decisions for optimal clinical data access performance. The health monitoring module 1920 may collect performance metrics from multiple system components. The health monitoring module 1920 may receive response time measurements from application programming interface connectors. The health monitoring module 1920 may receive response time measurements from user interface automation engines. The health monitoring module 1920 may receive error rate statistics from application programming interface connectors. The health monitoring module 1920 may receive error rate statistics from user interface automation engines.

The health monitoring module 1920 may calculate aggregate health metrics for each access pathway. The health monitoring module 1920 may compute average response times over configurable time windows. The health monitoring module 1920 may compute error rates as ratios of failed requests to total requests. The health monitoring module 1920 may track different error types separately. The health monitoring module 1920 may distinguish timeout errors from authentication failures. The health monitoring module 1920 may distinguish data validation errors from system unavailability errors.

The health monitoring module 1920 may maintain separate metric streams for different data types. The health monitoring module 1920 may track laboratory result retrieval performance independently from medication list retrieval performance. The health monitoring module 1920 may track imaging report access performance independently from clinical note access performance. The health monitoring module 1920 may enable fine-grained health assessment at the data type level.

The health monitoring module 1920 may compare current metrics against predefined threshold values. The health monitoring module 1920 may evaluate whether response times exceed acceptable limits. The health monitoring module 1920 may evaluate whether error rates exceed acceptable limits. The health monitoring module 1920 may determine when system health has degraded below operational standards. The health monitoring module 1920 may generate alert notifications when thresholds are exceeded.

The health monitoring module 1920 may transmit alert notifications to system administrators. The health monitoring module 1920 may transmit alert notifications to the routing engine 1910. The health monitoring module 1920 may trigger automated responses when critical thresholds are breached. The health monitoring module 1920 may initiate pathway switching procedures. The health monitoring module 1920 may activate failover mechanisms.

The health monitoring module 1920 may support configurable threshold values. The health monitoring module 1920 may allow administrators to define response time thresholds for different data types. The health monitoring module 1920 may allow administrators to define error rate thresholds for different operations. The health monitoring module 1920 may accommodate varying performance requirements across different clinical workflows.

The health monitoring module 1920 may store historical performance data. The health monitoring module 1920 may maintain time-series databases of response time measurements. The health monitoring module 1920 may maintain time-series databases of error rate statistics. The health monitoring module 1920 may maintain time-series databases of availability percentages. The health monitoring module 1920 may enable trend analysis over extended time periods.

The health monitoring module 1920 may analyze historical data to identify performance patterns. The health monitoring module 1920 may detect gradual performance degradation. The health monitoring module 1920 may identify recurring performance issues at specific times. The health monitoring module 1920 may recognize seasonal or cyclical performance variations. The health monitoring module 1920 may generate predictive alerts based on trend analysis.

The health monitoring module 1920 may provide early warning of developing issues. The health monitoring module 1920 may detect performance degradation before critical thresholds are reached. The health monitoring module 1920 may enable proactive intervention. The health monitoring module 1920 may allow administrators to address issues before workflow disruption occurs.

The health monitoring module 1920 may generate health status reports. The health monitoring module 1920 may compile current health metrics for all monitored pathways. The health monitoring module 1920 may compile historical performance summaries. The health monitoring module 1920 may transmit health status reports to a monitoring dashboard. The health monitoring module 1920 may update dashboard displays in real time.

The health monitoring module 1920 may provide visual health indicators. The health monitoring module 1920 may use color coding to represent health status levels. The health monitoring module 1920 may display green indicators for healthy pathways. The health monitoring module 1920 may display yellow indicators for degraded pathways. The health monitoring module 1920 may display red indicators for failed pathways.

The health monitoring module 1920 may enable administrators to drill down into detailed metrics. The health monitoring module 1920 may provide access to granular performance data for specific pathways. The health monitoring module 1920 may provide access to granular performance data for specific time periods. The health monitoring module 1920 may display individual request traces. The health monitoring module 1920 may show error details and stack traces.

The health monitoring module 1920 may track availability percentages. The health monitoring module 1920 may calculate uptime ratios for each access pathway. The health monitoring module 1920 may measure the proportion of time each pathway remains operational. The health monitoring module 1920 may generate availability reports for service level agreement compliance.

The health monitoring module 1920 may monitor connection pool health. The health monitoring module 1920 may track the number of active connections to external systems. The health monitoring module 1920 may track the number of idle connections in connection pools. The health monitoring module 1920 may detect connection pool exhaustion. The health monitoring module 1920 may alert when connection limits are approached.

The health monitoring module 1920 may measure data staleness. The health monitoring module 1920 may track the age of cached data. The health monitoring module 1920 may compare cached data timestamps against current time. The health monitoring module 1920 may generate freshness metrics. The health monitoring module 1920 may alert when cached data exceeds acceptable age limits.

The health monitoring module 1920 may monitor system resource utilization. The health monitoring module 1920 may track processor usage on application servers. The health monitoring module 1920 may track memory consumption on application servers. The health monitoring module 1920 may track network bandwidth utilization. The health monitoring module 1920 may detect resource constraints that may impact performance.

The health monitoring module 1920 may measure queue depths. The health monitoring module 1920 may track the number of pending requests in processing queues. The health monitoring module 1920 may detect queue buildup indicating processing bottlenecks. The health monitoring module 1920 may alert when queue depths exceed operational thresholds.

The health monitoring module 1920 may correlate metrics across multiple components. The health monitoring module 1920 may identify relationships between application programming interface performance and database query times. The health monitoring module 1920 may identify relationships between automation execution times and target system responsiveness. The health monitoring module 1920 may provide holistic system health assessment.

The health monitoring module 1920 may generate health score calculations. The health monitoring module 1920 may combine multiple metrics into composite health scores. The health monitoring module 1920 may weigh different metrics according to their relative importance. The health monitoring module 1920 may produce single numerical indicators of overall pathway health.

The health monitoring module 1920 may track health metric changes over time. The health monitoring module 1920 may calculate rates of change for response times. The health monitoring module 1920 may calculate rates of change for error rates. The health monitoring module 1920 may detect rapid degradation requiring immediate attention. The health monitoring module 1920 may distinguish transient issues from sustained problems.

The health monitoring module 1920 may implement anomaly detection algorithms. The health monitoring module 1920 may establish baseline performance profiles for each pathway. The health monitoring module 1920 may identify deviations from normal behavior patterns. The health monitoring module 1920 may flag unusual metric values even when thresholds are not exceeded. The health monitoring module 1920 may detect subtle performance anomalies.

The health monitoring module 1920 may categorize health events by severity. The health monitoring module 1920 may classify events as informational, warning, or critical. The health monitoring module 1920 may route events to appropriate notification channels based on severity. The health monitoring module 1920 may escalate critical events to on-call administrators.

The health monitoring module 1920 may maintain event history logs. The health monitoring module 1920 may record all health status changes. The health monitoring module 1920 may record all threshold violations. The health monitoring module 1920 may record all alert generations. The health monitoring module 1920 may provide audit trails for post-incident analysis.

The health monitoring module 1920 may support custom metric definitions. The health monitoring module 1920 may allow administrators to define additional performance indicators. The health monitoring module 1920 may allow administrators to specify custom calculation formulas. The health monitoring module 1920 may enable monitoring of institution-specific performance requirements.

The health monitoring module 1920 may integrate with external monitoring systems. The health monitoring module 1920 may export metrics to enterprise monitoring platforms. The health monitoring module 1920 may transmit health data to centralized logging systems. The health monitoring module 1920 may enable correlation with broader infrastructure monitoring.

The health monitoring module 1920 may provide application programming interfaces for metric access. The health monitoring module 1920 may expose current health metrics via programmatic interfaces. The health monitoring module 1920 may expose historical health data via programmatic interfaces. The health monitoring module 1920 may enable integration with custom dashboards and reporting tools.

The health monitoring module 1920 may perform continuous health checks. The health monitoring module 1920 may execute periodic test requests to verify pathway availability. The health monitoring module 1920 may measure response times for synthetic transactions. The health monitoring module 1920 may detect failures even during periods of low user activity.

The health monitoring module 1920 may track mean time to recovery. The health monitoring module 1920 may measure the duration of pathway outages. The health monitoring module 1920 may calculate average recovery times. The health monitoring module 1920 may generate reliability metrics for service level reporting.

The health monitoring module 1920 may monitor authentication success rates. The health monitoring module 1920 may track the proportion of successful authentication attempts. The health monitoring module 1920 may detect authentication service degradation. The health monitoring module 1920 may alert when authentication failures increase.

The health monitoring module 1920 may measure data retrieval completeness. The health monitoring module 1920 may track whether retrieved data contains all expected fields. The health monitoring module 1920 may detect partial data returns. The health monitoring module 1920 may alert when data completeness falls below acceptable levels.

The health monitoring module 1920 may provide health status application programming interfaces for the routing engine 1910. The health monitoring module 1920 may respond to real-time health queries from routing components. The health monitoring module 1920 may supply current pathway health scores. The health monitoring module 1920 may enable routing decisions based on up-to-date health information.

The health monitoring module 1920 may generate periodic health summary reports. The health monitoring module 1920 may compile daily performance summaries. The health monitoring module 1920 may compile weekly performance summaries. The health monitoring module 1920 may compile monthly performance summaries. The health monitoring module 1920 may distribute reports to stakeholders.

The health monitoring module 1920 may track service level agreement compliance. The health monitoring module 1920 may measure actual performance against defined service level objectives. The health monitoring module 1920 may calculate compliance percentages. The health monitoring module 1920 may generate service level agreement violation reports.

The health monitoring module 1920 may support multiple monitoring granularities. The health monitoring module 1920 may collect metrics at one-second intervals for real-time monitoring. The health monitoring module 1920 may aggregate metrics at one-minute intervals for operational dashboards. The health monitoring module 1920 may aggregate metrics at one-hour intervals for trend analysis. The health monitoring module 1920 may balance monitoring precision with storage efficiency.

### C. A Data Normalization Module

The data normalization module 1930 may receive clinical data retrieved through the selected pathway. The module may identify the source format of the received data. The module may determine the schema used by the source system. The module may extract individual data elements from the received data payload. The module may map each extracted data element to a corresponding field in a unified data schema.

The data normalization module 1930 may include a schema mapping engine. The schema mapping engine may maintain a mapping table that associates source system schemas with the unified schema. The schema mapping engine may identify field correspondences between heterogeneous data structures. The schema mapping engine may apply transformation rules to convert source field formats to unified field formats. The schema mapping engine may handle nested data structures by recursively mapping child elements.

The data normalization module 1930 may include a terminology translation component. The terminology translation component may identify clinical terminology codes present in the source data. The terminology translation component may determine the coding system used by the source system. The terminology translation component may access a terminology mapping database containing cross-references between coding systems. The terminology translation component may translate source codes to standardized codes such as SNOMED CT, LOINC, or RxNorm. The terminology translation component may preserve the original source code alongside the translated code for provenance purposes.

The data normalization module 1930 may include a unit conversion processor. The unit conversion processor may identify units of measure associated with quantitative clinical values. The unit conversion processor may determine whether unit conversion is required based on institutional standards. The unit conversion processor may apply mathematical conversion factors to transform values from source units to standard units. The unit conversion processor may maintain the original unit value and unit designation for audit purposes. The unit conversion processor may validate converted values against clinically plausible ranges.

The data normalization module 1930 may include a timestamp normalization component. The timestamp normalization component may extract temporal information from source data. The timestamp normalization component may identify the timezone associated with source timestamps. The timestamp normalization component may convert timestamps to a standardized format such as ISO 8601. The timestamp normalization component may convert timestamps to a common timezone such as UTC. The timestamp normalization component may preserve the original timestamp and timezone information for provenance tracking.

The data normalization module 1930 may include a data validation engine. The data validation engine may verify that required data fields are present in the normalized data. The data validation engine may check data types of normalized fields against expected types. The data validation engine may validate data formats using regular expressions or format specifications. The data validation engine may assess clinical plausibility of quantitative values against acceptable ranges. The data validation engine may flag data elements that fail validation checks for review.

The data normalization module 1930 may include a provenance metadata attachment component. The provenance metadata attachment component may generate metadata describing the source of each data element. The provenance metadata attachment component may record the identifier of the source system from which data was retrieved. The provenance metadata attachment component may record the access method used to retrieve the data. The provenance metadata attachment component may record the timestamp when the data was retrieved from the source system. The provenance metadata attachment component may calculate a data freshness indicator based on the retrieval timestamp and current time. The provenance metadata attachment component may embed the provenance metadata within the normalized data structure.

The data normalization module 1930 may include a data integrity checker. The data integrity checker may compute checksums or hash values for normalized data elements. The data integrity checker may compare computed checksums against expected values to detect data corruption. The data integrity checker may verify referential integrity between related data elements. The data integrity checker may detect duplicate data elements that may have been retrieved from multiple sources. The data integrity checker may resolve conflicts between duplicate data elements based on precedence rules.

The data normalization module 1930 may include a structured output generator. The structured output generator may format normalized data according to a standardized output schema. The structured output generator may serialize normalized data into a specified format such as JSON or XML. The structured output generator may organize data elements into logical groupings for presentation. The structured output generator may apply sorting or filtering rules to the normalized data. The structured output generator may generate a complete normalized data package ready for caching or presentation.

The data normalization module 1930 may include a cache storage interface. The cache storage interface may transmit normalized data to a caching subsystem for storage. The cache storage interface may generate a cache key based on data type and identifying parameters. The cache storage interface may specify an expiration time or time-to-live value for the cached data. The cache storage interface may confirm successful storage of normalized data in the cache. The cache storage interface may handle cache storage errors by logging failures and notifying upstream components.

The data normalization module 1930 may include a presentation layer interface. The presentation layer interface may transmit normalized data to presentation components for rendering. The presentation layer interface may format data according to presentation layer requirements. The presentation layer interface may include provenance indicators in the transmitted data package. The presentation layer interface may provide data freshness information for display to end users. The presentation layer interface may support real-time streaming of normalized data as it becomes available.

The data normalization module 1930 may include an error handling component. The error handling component may detect errors during the normalization process. The error handling component may classify errors by type and severity. The error handling component may log detailed error information for troubleshooting. The error handling component may generate error notifications to upstream components. The error handling component may implement retry logic for transient errors. The error handling component may provide fallback behavior when normalization cannot be completed.

The data normalization module 1930 may include a performance optimization component. The performance optimization component may cache frequently used mapping tables in memory. The performance optimization component may parallelize normalization operations for large data sets. The performance optimization component may prioritize normalization of high-priority data elements. The performance optimization component may monitor normalization throughput and latency. The performance optimization component may adjust processing strategies based on performance metrics.

### D. An Intent Processing Module

In some embodiments, the system 1900 may optionally include an intent processing module 1940. The intent processing module 1940 may receive natural language input from users through the presentation layer interface. The module may parse the received input to extract semantic meaning from the text. The parsing process may involve tokenization of the input string into individual words or phrases. The module may perform part-of-speech tagging to identify grammatical roles of words within the input. The module may conduct syntactic analysis to understand the structural relationships between words and phrases.

The intent processing module 1940 may classify the parsed input to determine the user's intended action. The classification process may compare the parsed input against a database of whitelisted intents. Each whitelisted intent may be defined by a set of linguistic patterns that characterize that intent. The module may use pattern matching algorithms to identify correspondence between the input and whitelisted intent patterns. The module may generate a confidence score for each potential intent match. The confidence score may indicate the likelihood that the identified intent correctly represents the user's intention.

The intent processing module 1940 may validate that the identified intent exists within the whitelist of approved intents. The whitelist may contain only those intents that have been explicitly approved for use within the system. The module may reject any natural language input that does not correspond to a whitelisted intent. When an input is rejected, the module may generate an error message indicating that the request cannot be processed. The module may provide guidance to the user on how to rephrase the request in a form that maps to an approved intent.

The intent processing module 1940 may extract parameters from the natural language input. Parameters may include patient identifiers that specify which patient's data should be accessed. Parameters may include date ranges that define temporal boundaries for data retrieval. Parameters may include data types that specify which category of clinical information is being requested. The module may use named entity recognition techniques to identify parameter values within the input text. The module may validate extracted parameters to ensure they conform to expected formats. The module may check that parameter values fall within acceptable ranges.

The intent processing module 1940 may handle situations where required parameters are missing from the natural language input. The module may identify which parameters are required for the identified intent. The module may determine which required parameters were not provided in the input. The module may generate a prompt requesting the user to provide the missing information. The prompt may specify which parameter is needed and may provide examples of acceptable values. The module may wait for the user to provide the missing parameter before proceeding with intent processing.

The intent processing module 1940 may resolve ambiguous natural language inputs. The module may detect when an input could map to multiple whitelisted intents with similar confidence scores. The module may generate a list of possible interpretations of the ambiguous input. The module may present the list of interpretations to the user through the presentation layer. The module may request that the user select the correct interpretation from the presented options. The module may receive the user's selection and may proceed with the selected intent.

The intent processing module 1940 may utilize context information to improve intent classification accuracy. The module may access the current user context from the context manager. The context may include the currently active patient. The context may include the current workflow phase. The context may include recently performed actions. The module may use context information to disambiguate inputs that reference implicit subjects. The module may use context to resolve pronouns or relative references in the natural language input.

The intent processing module 1940 may decompose complex intents into sequences of primitive operations. A complex intent may require multiple system actions to fulfill. The module may analyze the intent structure to identify constituent operations. Each primitive operation may correspond to a single data retrieval action or system command. The module may order the primitive operations in a logical sequence. The module may validate each primitive operation before adding it to the sequence. The validation may ensure that each operation complies with security policies and user permissions.

The intent processing module 1940 may convert validated intents into structured request objects. A structured request object may contain the intent type identifier. The request object may contain all extracted parameters with their values. The request object may contain metadata about the original natural language input. The metadata may include the original input text. The metadata may include the confidence score of the intent classification. The metadata may include the timestamp when the input was received. The module may transmit the structured request object to the routing engine for execution.

The intent processing module 1940 may maintain an audit trail of all natural language interactions. The module may log each natural language input received from users. The audit record may include the original input text exactly as entered by the user. The audit record may include the identified intent type. The audit record may include all extracted parameters and their values. The audit record may include the confidence score of the intent classification. The audit record may include any errors or rejections that occurred during processing. The audit record may include the timestamp of the interaction. The audit record may include the user identity associated with the input.

The intent processing module 1940 may provide feedback to users about intent processing results. The module may generate confirmation messages when an intent is successfully identified and validated. The confirmation message may restate the interpreted intent in structured form. The confirmation message may display the extracted parameters for user verification. The module may request explicit user confirmation before proceeding with certain high-risk intents. High-risk intents may include write operations or actions that modify clinical data. The module may wait for user confirmation before transmitting the intent to the routing engine.

The intent processing module 1940 may support multiple natural language input modalities. The module may receive typed text input from keyboard entry. The module may receive transcribed speech input from voice recognition systems. The module may apply the same intent processing logic regardless of input modality. The module may handle modality-specific characteristics such as speech recognition errors. The module may apply error correction algorithms to improve accuracy of transcribed speech input.

The intent processing module 1940 may learn from user interactions to improve intent classification over time. The module may track which intents are most frequently used by each user. The module may track which parameter values are most commonly provided. The module may use this usage data to adjust confidence scoring for future classifications. The module may prioritize frequently used intents when multiple intents have similar confidence scores. The module may suggest parameter values based on historical usage patterns.

The intent processing module 1940 may enforce role-based constraints on intent availability. The module may check the user's role before allowing access to certain intents. Some intents may be restricted to specific user roles such as physicians or administrators. The module may reject intents that the current user's role is not authorized to use. The rejection message may indicate that the requested action requires elevated permissions. The module may log unauthorized intent attempts in the security audit log.

The intent processing module 1940 may support intent chaining for complex workflows. A user may provide a sequence of related natural language inputs. The module may recognize that subsequent inputs are related to previous intents. The module may maintain state information across multiple related intents. The module may use information from previous intents to inform processing of subsequent intents. The module may allow users to reference results from previous intents in new inputs.

The intent processing module 1940 may provide intent suggestion capabilities. The module may analyze the current context to predict likely user intents. The module may generate suggestions for intents that are commonly used in the current context. The module may display suggested intents to the user through the presentation layer. The user may select a suggested intent rather than typing a natural language input. The module may pre-populate parameters for suggested intents based on context.

The intent processing module 1940 may handle negation and modification in natural language input. The module may detect negation words such as “not” or “except” in the input. The module may modify the intent interpretation based on detected negation. The module may detect modification words such as “only” or “all” that change the scope of the intent. The module may adjust extracted parameters based on detected modifiers. The module may ensure that negation and modification are correctly reflected in the structured request object.

The intent processing module 1940 may support multi-language natural language input. The module may detect the language of the input text. The module may apply language-specific parsing and classification models. The module may maintain separate whitelists for different languages. The module may translate intents from one language to a common internal representation. The module may generate feedback messages in the same language as the input.

The intent processing module 1940 may validate intent execution prerequisites. The module may check that all required system components are available before transmitting an intent for execution. The module may verify that target data sources are accessible. The module may check that the user has an active session with required permissions. The module may reject intents when prerequisites are not met. The module may provide informative error messages explaining which prerequisites are missing.

The intent processing module 1940 may implement timeout mechanisms for intent processing. The module may set a maximum time limit for intent classification and parameter extraction. The module may abort processing if the time limit is exceeded. The module may return a timeout error to the user when processing cannot complete within the time limit. The module may log timeout events for performance monitoring and optimization.

### E. A Provider Profile Module

In some embodiments, the system 1900 may optionally include a provider profile module 1950. The provider profile module 1950 may maintain persistent user-specific configurations that transcend individual electronic health record systems. The module may associate configuration data with a provider identity rather than with any particular system-specific user account. The provider identity may serve as a global identifier that remains constant across multiple healthcare information systems. The module may store interaction preferences that define how the provider prefers to interact with clinical data regardless of which underlying system contains that data.

The provider profile module 1950 may maintain layout preference data for each provider. The layout preference data may specify the arrangement of user interface panels on the display screen. The layout preference data may specify which panels are visible by default when the provider accesses patient information. The layout preference data may specify the relative sizes of different panels within the interface. The layout preference data may specify the positioning of panels relative to one another. The module may apply these layout preferences consistently whenever the provider accesses the platform, regardless of which underlying electronic health record system is being accessed.

The provider profile module 1950 may store workflow configuration data for each provider. The workflow configuration data may define sequences of views or actions that the provider commonly performs. A workflow configuration may specify a series of data views that the provider typically accesses in a particular order when performing a specific clinical task. The workflow configuration may define default filter settings that should be applied when displaying certain types of clinical data. The workflow configuration may specify time ranges or date filters that should be applied by default to laboratory results or medication lists.

The provider profile module 1950 may maintain presentation default settings for each provider. The presentation defaults may specify how clinical data should be formatted when displayed to the provider. The presentation defaults may specify whether laboratory results should be displayed in tabular format or graphical format. The presentation defaults may specify the sort order for medication lists. The presentation defaults may specify which data elements should be prominently highlighted in patient summary views. The presentation defaults may specify color coding preferences for abnormal values or critical alerts.

The provider profile module 1950 may store notification preference data for each provider. The notification preferences may specify which types of clinical alerts the provider wishes to receive. The notification preferences may specify the delivery method for alerts, such as on-screen notifications, email messages, or text messages. The notification preferences may specify the urgency threshold for alerts that should interrupt the provider's current workflow. The notification preferences may specify quiet hours during which non-urgent notifications should be suppressed.

The provider profile module 1950 may maintain a history of the provider's interaction patterns. The interaction history may record which data types the provider accesses most frequently. The interaction history may record which natural language commands the provider uses most often. The interaction history may record which workflow sequences the provider performs regularly. The module may analyze this interaction history to identify patterns in the provider's behavior. The module may use identified patterns to generate personalized suggestions or recommendations.

The provider profile module 1950 may map a single provider identity to multiple local user accounts. A healthcare provider may have separate user accounts in different electronic health record systems operated by different facilities or departments within a healthcare organization. The module may maintain associations between the provider's global identity and each of their local system-specific identities. The associations may include the local username or user identifier for each system. The associations may include credential information or authentication tokens for each system. The associations may specify which facilities or departments are associated with each local account.

The provider profile module 1950 may apply the provider's global profile consistently across all mapped local accounts. When the provider accesses any electronic health record system through the platform, the module may retrieve the provider's global profile. The module may apply the layout preferences from the global profile to the current session. The module may apply the workflow configurations from the global profile to the current session. The module may apply the presentation defaults from the global profile to the current session. This consistent application may occur regardless of which underlying electronic health record system is currently being accessed.

The provider profile module 1950 may synchronize profile data across multiple devices and sessions. When a provider updates their preferences or saves a new configuration, the module may propagate these changes to all active sessions associated with that provider. The module may store the updated profile data persistently in a profile database. The module may ensure that the updated profile is available in future sessions on any device. The synchronization may occur in real time or near-real time to maintain consistency across concurrent sessions.

The provider profile module 1950 may support profile versioning to enable recovery of previous configurations. When a provider makes changes to their profile, the module may save the previous version of the profile before applying the changes. The module may assign a version number or timestamp to each saved profile version. The module may maintain a history of profile versions for a configurable retention period. The provider may request to view previous profile versions. The provider may select a previous profile version to restore if they are not satisfied with recent changes.

The provider profile module 1950 may enable administrators to define default profiles for different user roles. A default profile may specify standard configurations that are appropriate for a particular role within the healthcare organization. The default profile for a physician role may include different layout preferences than the default profile for a nurse role. The default profile for a pharmacist role may include different workflow configurations than the default profile for a laboratory technician role. When a new user account is created, the module may initialize the user's profile with the default settings for their assigned role. The user may subsequently customize their profile to suit their individual preferences while retaining the role-appropriate defaults as a baseline.

The provider profile module 1950 may store frequently used action sequences for each provider. An action sequence may represent a series of system operations that the provider performs regularly. The module may record when the provider performs a particular sequence of actions multiple times. The module may identify recurring action patterns through analysis of the interaction history. The module may save identified action sequences as shortcuts that can be invoked with a single command. The provider may assign names or labels to saved action sequences for easy identification and retrieval.

The provider profile module 1950 may maintain favorite data views for each provider. A favorite data view may represent a specific configuration of filters, sort orders, and visible columns for a particular type of clinical data. The provider may save a customized laboratory results view as a favorite. The provider may save a customized medication list view as a favorite. The module may store the complete configuration of each favorite view. The provider may quickly switch to a favorite view by selecting it from a list of saved favorites. The module may apply all associated filters and settings when a favorite view is activated.

The provider profile module 1950 may track the provider's preferred terminology and language settings. The terminology preferences may specify which clinical terminology system the provider prefers to see. The terminology preferences may specify whether the provider prefers to see generic drug names or brand names for medications. The language settings may specify the provider's preferred language for user interface elements and system messages. The module may apply these preferences when displaying clinical data and user interface text.

The provider profile module 1950 may store the provider's accessibility preferences. The accessibility preferences may specify font sizes for improved readability. The accessibility preferences may specify high-contrast color schemes for users with visual impairments. The accessibility preferences may specify whether screen reader support should be enabled. The accessibility preferences may specify keyboard navigation preferences for users who prefer not to use a mouse. The module may apply these accessibility settings consistently across all sessions and devices.

The provider profile module 1950 may maintain the provider's data freshness tolerance settings. The freshness tolerance settings may specify how old cached data can be before the provider requires a refresh from the primary source. Different data types may have different freshness tolerance thresholds. Laboratory results may have a shorter freshness tolerance than demographic information. The module may use these settings to determine when cached data is acceptable and when live data retrieval is required.

The provider profile module 1950 may enable providers to share profile elements with colleagues. A provider may designate certain profile elements as shareable. Another provider with appropriate permissions may access and apply the shared profile elements to their own profile. This sharing capability may facilitate standardization of workflows within a clinical team. This sharing capability may enable experienced providers to share optimized configurations with less experienced colleagues. The module may maintain access controls to ensure that only authorized users can access shared profile elements.

The provider profile module 1950 may support profile templates that can be applied to multiple users. An administrator may create a profile template that defines a standard set of configurations. The template may be applied to all users within a particular department or specialty. The template may serve as a starting point that users can further customize. The module may track which users have applied which templates. The module may enable administrators to update templates and optionally propagate updates to users who have applied those templates.

The provider profile module 1950 may maintain the provider's search history and frequently accessed patients. The module may record which patients the provider accesses most frequently. The module may record which search terms the provider uses most often when looking for patients or clinical information. The module may use this history to provide quick access shortcuts to frequently accessed patients. The module may use this history to improve search result ranking by prioritizing results that match the provider's typical access patterns.

The provider profile module 1950 may store the provider's documentation preferences. The documentation preferences may specify which clinical note templates the provider prefers to use. The documentation preferences may specify default values for certain documentation fields. The documentation preferences may specify whether the provider prefers to dictate notes or type them. The documentation preferences may specify formatting preferences for generated documentation. The module may apply these preferences when the provider creates new clinical documentation.

The provider profile module 1950 may maintain the provider's alert acknowledgment history. The module may record which types of alerts the provider typically acknowledges quickly and which types the provider tends to dismiss. The module may use this history to adjust alert presentation or prioritization. The module may identify alert types that the provider consistently ignores and may suggest adjusting notification settings for those alert types.

The provider profile module 1950 may enable providers to export their profile data. The provider may request an export of their complete profile configuration. The module may generate an export file containing all profile settings in a portable format. The exported profile may be imported into another instance of the platform. This export capability may facilitate profile migration when a provider moves to a different healthcare organization that uses the same platform.

The provider profile module 1950 may implement profile security measures. The module may encrypt sensitive profile data when stored in the profile database. The module may require authentication before allowing profile modifications. The module may log all changes to provider profiles in an audit trail. The audit trail may record who made changes, what was changed, and when the changes occurred. The module may implement access controls to prevent unauthorized access to provider profile data.

The provider profile module 1950 may provide a profile management interface. The interface may enable providers to view and edit their profile settings. The interface may organize profile settings into logical categories such as layout, workflow, notifications, and accessibility. The interface may provide preview functionality that shows how changes will affect the user interface before the changes are applied. The interface may provide a reset option that restores default settings for any profile element.

The provider profile module 1950 may support profile inheritance for hierarchical organizations. A profile setting defined at an organizational level may be inherited by all users within that organization. A profile setting defined at a department level may override the organizational setting for users in that department. An individual user's profile setting may override both organizational and departmental settings. This hierarchical approach may enable centralized management of certain settings while preserving individual customization flexibility.

The provider profile module 1950 may maintain statistics about profile usage. The module may track how often each profile element is used or modified. The module may identify profile elements that are rarely used and may suggest removing them to simplify the profile. The module may identify profile elements that are frequently modified and may suggest creating shortcuts or favorites for those elements. The module may generate reports showing profile usage patterns across the provider population.

### III. Platform Operation

FIG. 13A and FIG. 13B are flow charts setting forth the general stages involved in a method 1300 consistent with an embodiment of the disclosure for providing a method for recommending treatment plans, preventive actions, and preparedness actions utilizing a platform 100. Method 1300 may be implemented using a computing device 1500 as described in more detail below with respect to FIG. 15.

Although method 1300 has been described to be performed by computing device 1500, it should be understood that, in some embodiments, different operations may be performed by different networked elements in operative communication with computing device 1500. For example, server 110 and/or computing device 1500 may be employed in the performance of some or all of the stages in method 1300. Moreover, server 110 may be configured much like computing device 1500 and, in some instances, be one and the same embodiment. Similarly, platform apparatus 100 may be employed in the performance of some or all of the stages in method 1300. Apparatus 100 may also be configured much like computing device 1500.

Although method 1400 has been described to be performed by platform 100, it should be understood that computing device 1500 may be used to perform the various stages of method 1400. Furthermore, in some embodiments, different operations may be performed by different networked elements in operative communication with computing device 1500. For example, server 110 may be employed in the performance of some or all of the stages in method 1400. Moreover, server 110 may be configured much like computing device 1500. Similarly, platform apparatus 100 may be employed in the performance of some or all of the stages in method 1400. Apparatus 100 may also be configured much like computing device 1500.

Although method 1600 has been described to be performed by platform 100, it should be understood that computing device 1500 may be used to perform the various stages of method 1600. Furthermore, in some embodiments, different operations may be performed by different networked elements in operative communication with computing device 1500. For example, server 110 may be employed in the performance of some or all of the stages in method 1600. Moreover, server 110 may be configured much like computing device 1500. Similarly, platform apparatus 100 may be employed in the performance of some or all of the stages in method 1600. Apparatus 100 may also be configured much like computing device 1500.

Although method 1700 has been described to be performed by platform 100, it should be understood that computing device 1500 may be used to perform the various stages of method 1700. Furthermore, in some embodiments, different operations may be performed by different networked elements in operative communication with computing device 1500. For example, server 110 may be employed in the performance of some or all of the stages in method 1700. Moreover, server 110 may be configured much like computing device 1500. Similarly, platform apparatus 100 may be employed in the performance of some or all of the stages in method 1700. Apparatus 100 may also be configured much like computing device 1500.

Although method 1800 has been described to be performed by platform 100, it should be understood that computing device 1500 may be used to perform the various stages of method 1800. Furthermore, in some embodiments, different operations may be performed by different networked elements in operative communication with computing device 1500. For example, server 110 may be employed in the performance of some or all of the stages in method 1800. Moreover, server 110 may be configured much like computing device 1500. Similarly, platform apparatus 100 may be employed in the performance of some or all of the stages in method 1800. Apparatus 100 may also be configured much like computing device 1500.

Although the stages illustrated by the flow charts are disclosed in a particular order, it should be understood that the order is disclosed for illustrative purposes only. Stages may be combined, separated, reordered, and various intermediary stages may exist. Accordingly, it should be understood that the various stages illustrated within the flow chart may be, in various embodiments, performed in arrangements that differ from the ones illustrated. Moreover, various stages may be added or removed from the flow charts without altering or deterring from the fundamental scope of the depicted methods and systems disclosed herein. Ways to implement the stages of method 1300 will be described in greater detail below.

Method 1300 may begin at starting block 1300 and proceed to stage 1302 where computing device 1500 may provide a first interface for receiving data points associated with a patient encounter. For example, a user interface module may provide an interface for entry of practitioner notes associated with a patient encounter. In general, the first flow chart describes an embodiment of the disclosure in terms of computing device 1500, programming modules 1506 (i.e., Application Modules 1520), and the associated method.

From stage 1304, where computing device 1500 receives data points from a user in an input tier of the first interface, method 1300 may advance to stage 1306 where computing device 1500 may provide a second interface to assist with data entry into the first interface.

Once computing device 1500 provides a second interface to assist with data entry into the first interface in stage 1306, method 1300 may continue to stage 1308 where computing device 1500 may make a plurality of suggested data points available for the user to select and enter data into the first interface.

After computing device 1500 make a plurality of suggested data points available for the user to select and enter data into the first interface in stage 1308, method 1300 may proceed to stage 1310 where computing device 1500 may receive at least one data point or data element from various data sources. For example, data may be received from internal patient data or external data sources.

After computing device 1500 receives at least one data point or data element from various data sources in stage 1310, method 1300 may proceed to stage 1312 where computing device 1500 may analyze the data, data point, or data element.

After computing device 1500 analyzes the data, data point, or data element in stage 1312, method 1300 may proceed to stage 1314 where computing device 1500 may recommend a diagnostic question, treatment, action or other option based on analysis performed.

After computing device 1500 recommends a diagnostic question, treatment, action or other option based on analysis performed in stage 1314, method 1300 may proceed to stage 1316 where computing device 1500 may determine the probable impact of an event occurrence on a patient population. For example, will a probable high pollen count, high smog level, or poor air quality alert adversely affect the patient population associated with the practitioners.

After computing device 1500 determines the probable impact of an event occurrence on a patient population in stage 1316, method 1300 may proceed to stage 1318 where computing device 1500 may analyze the probable impact to determine whether one or more patients or practitioners qualifies to receive an alert for a preventive or preparedness activity. For example, if a patient with asthma is near an area where there is poor air quality, said patient would qualify to receive an alert. Another asthma patient who is a safe determined distance away from the area of poor air quality would not qualify to receive an alert. This patient would qualify if there is a change in their location and they approach a determined radius near the area of poor air quality.

Once computing device 1500 analyzes the probable impact to determine whether one or more patients or practitioners qualifies to receive an alert for a preventive or preparedness activity in stage 1318, method 1300 may then end at stage 1320 or stage 1322 where computing device 1500 may provide a patient with a preventive action, suggestion, recommendation, or other option (1320) or provide a practitioner with a preparedness action, suggestion, recommendation, or other option (1322).

Although the stages illustrated by the flow charts are disclosed in a particular order, it should be understood that the order is disclosed for illustrative purposes only. Stages may be combined, separated, reordered, and various intermediary stages may exist. Accordingly, it should be understood that the various stages illustrated within the flow chart may be, in various embodiments, performed in arrangements that differ from the ones illustrated. Moreover, various stages may be added or removed from the flow charts without altering or deterring from the fundamental scope of the depicted methods and systems disclosed herein. Ways to implement the stages of method 1400 will be described in greater detail below.

Method 1400 may begin at starting block 1400 and proceed to stage 1402 where computing device 1500 may input data associated with a patient encounter. From stage 1402, where computing device 1500 inputs data associated with a patient encounter, method 1400 may advance to stage 1404 where computing device 1500 may determine at least one patient profile correlating to another patient profile or similar patient profile.

Once computing device 1500 determines at least one patient profile correlating to another patient profile or similar patient profile in stage 1404, method 1400 may continue to stage 1406 where computing device 1500 may analyze data associated with patient data, encounter note, or another patient profile. Once computing device 1500 analyze data associated with patient data, encounter note, or another patient profile in stage 1406, method 1400 may then end at stage 1408 where computing device 1500 provides a recommended treatment, diagnosis, or other data point.

Although the stages illustrated by the flow charts are disclosed in a particular order, it should be understood that the order is disclosed for illustrative purposes only. Stages may be combined, separated, reordered, and various intermediary stages may exist. Accordingly, it should be understood that the various stages illustrated within the flow chart may be, in various embodiments, performed in arrangements that differ from the ones illustrated. Moreover, various stages may be added or removed from the flow charts without altering or deterring from the fundamental scope of the depicted methods and systems disclosed herein. Ways to implement the stages of method 1600 will be described in greater detail below.

Method 1600 may begin at starting block 1600 and proceed to stage 1602 where computing device 1500 may determine whether an occurrence of an event would adversely impact patients within the patient population. For example, will a probable high pollen count, high smog level, or poor air quality alert adversely affect the patient population associated with the practitioner(s).

From stage 1602, where computing device 1500 determines whether an occurrence of an event would adversely impact patients within the patient population, method 1600 may advance to stage 1604 where computing device 1500 may determine whether a probability of an occurrence of an event has reached a predetermined threshold. For example, if there is a seventy-five percent chance of a high pollen count when sixty percent is the predetermined threshold, the probability of a high pollen count has reached a predetermined threshold.

Once computing device 1500 determine whether a probability of an occurrence of an event has reached a predetermined threshold in stage 1604, method 1600 may continue to stage 1606 where computing device 1500 may alert qualified patient(s) or practitioner(s) regarding the high probability of occurrence of the adverse event. For example, if a patient with asthma is near an area where there is poor air quality, said patient would qualify to receive an alert. Another asthma patient who is a safe determined distance away from the area of poor air quality would not qualify to receive an alert. This patient would qualify if there is a change in their location and they approach a determined radius near the area of poor air quality. A practitioner specializing in respiratory care of asthma patients would also qualify for an alert or notification.

After computing device 1500 alert qualified patients or practitioners regarding the high probability of occurrence of the adverse event in stage 1606, method 1600 may proceed to stage 1608 where computing device 1500 may predict a preventive or preparedness action. Once computing device 1500 predicts a preventive or preparedness action in stage 1608, method 1600 may then end at stage 1610 where computing device 1500 may provide the patient(s) or practitioner(s) with a preventive or preparedness action. For example, if a patient with asthma is near an area where there is poor air quality, said patient would receive a preventive action such as but not limited to, “avoid the area” or “reroute your travel around the area.” A practitioner specializing in respiratory care of asthma patients would also receive a preparedness action such as but not limited to, “prepare for a high number of walk-ins” or “order more asthma inhalers and respiratory treatment supplies.”

Although the stages illustrated by the flow charts are disclosed in a particular order, it should be understood that the order is disclosed for illustrative purposes only. Stages may be combined, separated, reordered, and various intermediary stages may exist. Accordingly, it should be understood that the various stages illustrated within the flow chart may be, in various embodiments, performed in arrangements that differ from the ones illustrated. Moreover, various stages may be added or removed from the flow charts without altering or deterring from the fundamental scope of the depicted methods and systems disclosed herein. Ways to implement the stages of method 1700 will be described in greater detail below.

Method 1700 may begin at starting block 1700 and proceed to stage 1702 where computing device 1500 may receive at least one data element. From stage 1702, where computing device 1500 receives at least one data element, method 1700 may advance to stage 1704 where computing device 1500 may analyze the data element. For example, the favorites module, recommendation module, patient tracking module, or predictive analytics module may analyze the data element. Once computing device 1500 analyzes the data element in stage 1704, method 1700 may then end at stage 1706 where computing device 1500 may make a recommendation based on the analysis. For example, the recommendation module may recommend a diagnostic question, recommended treatment, or recommended action.

Although the stages illustrated by the flow charts are disclosed in a particular order, it should be understood that the order is disclosed for illustrative purposes only. Stages may be combined, separated, reordered, and various intermediary stages may exist. Accordingly, it should be understood that the various stages illustrated within the flow chart may be, in various embodiments, performed in arrangements that differ from the ones illustrated. Moreover, various stages may be added or removed from the flow charts without altering or deterring from the fundamental scope of the depicted methods and systems disclosed herein. Ways to implement the stages of method 1800 will be described in greater detail below.

Method 1800 may begin at starting block 1800 and proceed to stage 1802 where computing device 1500 may receive data points from a patient entered by a patient, practitioner, or user. For example, a user interface module may provide an interface for entry of practitioner notes associated with a patient encounter. From stage 1802, where computing device 1500 receives data points from a user in an input tier, method 1800 may advance to stage 1804 where computing device 1500 may receive treatment notes for a patient.

Once computing device 1500 receives treatment notes for a patient in stage 1804, method 1800 may continue to stage 1806 where computing device 1500 may specify patient data points in a first tier relating to reasons for visit or chief complaints. After computing device 1500 specifies patient data points in a first tier relating to reasons for visit or chief complaints in stage 1806, method 1800 may proceed to stage 1808 where computing device 1500 may specify patient data points in a second tier relating to history reasons for visit or history of present illness

After computing device 1500 specifies patient data points in a second tier relating to history reasons for visit or history of present illness in stage 1808, method 1800 may proceed to stage 1810 where computing device 1500 specify a body system in a third tier relating to the first tier or second tier. After computing device 1500 specifies a body system in a third tier relating to the first tier or second tier in stage 1810, method 1800 may proceed to stage 1812 where computing device 1500 may specify a fourth tier, a condition associated with the body system specified in the third tier.

After computing device 1500 specifies a fourth tier a condition associated with the body system specified in the third tier in stage 1812, method 1800 may proceed to stage 1814 where computing device 1500 may specify a treatment plan in a fifth tier. Once computing device 1500 specifies a treatment plan in stage 1814, method 1800 may then end at stage 1816 where computing device 1500 may specify a treatment in a sixth tier.

Consistent with embodiments of the present disclosure, a method for integrating clinical data across heterogeneous electronic health record systems may be performed by at least one of the aforementioned modules. The method may be embodied as, for example, but not limited to, computer instructions, which, when executed, perform the method.

The method may provide a comprehensive approach for integrating clinical data across heterogeneous electronic health record systems by dynamically selecting optimal data access pathways based on real-time performance conditions. The method begins by receiving a request for clinical data from an EHR system and determining the availability of both application programming interface (API) and user interface automation mechanisms for accessing the requested information. The processor continuously monitors real-time health metrics including response times and error rates for both access mechanisms, then intelligently selects the most appropriate pathway based on current performance conditions. Once the optimal mechanism is selected, the method executes the data request, retrieves the clinical information, and normalizes it into a standardized format to ensure consistency across different EHR platforms. Finally, the method annotates the normalized clinical data with comprehensive provenance metadata that includes details about the selected access mechanism and retrieval timestamp, providing transparency and traceability for the data access process while ensuring clinicians have reliable access to patient information regardless of underlying system performance variations.

The clinical data integration system may provide significant technological improvements over conventional healthcare information systems. Traditional electronic health record systems may rely on static data access pathways that cannot adapt to changing system performance conditions, resulting in failed data retrievals during peak usage periods or system maintenance windows. The dynamic routing engine may solve this technical problem by implementing real-time performance monitoring algorithms that continuously evaluate pathway health metrics and automatically switch between access mechanisms to maintain optimal data retrieval performance. This technological solution may reduce system downtime impact by up to 90% compared to conventional static routing approaches.

The routing engine may implement sophisticated load balancing algorithms that distribute data access requests across multiple pathways based on current system capacity and response characteristics. For example, when API response times exceed predetermined thresholds due to database query optimization processes, the routing engine may automatically redirect subsequent requests to user interface automation pathways that access cached data representations. This technical approach may prevent cascade failures that occur in conventional systems when a single access pathway becomes overloaded, thereby improving overall system reliability and clinical workflow continuity.

The health monitoring module may employ machine learning algorithms to predict pathway performance degradation before complete failures occur. By analyzing historical performance patterns, network latency trends, and system resource utilization metrics, the monitoring module may proactively trigger pathway switches to maintain consistent data access performance. This predictive capability may represent a significant advancement over reactive monitoring systems that only respond to failures after they have already impacted clinical operations.

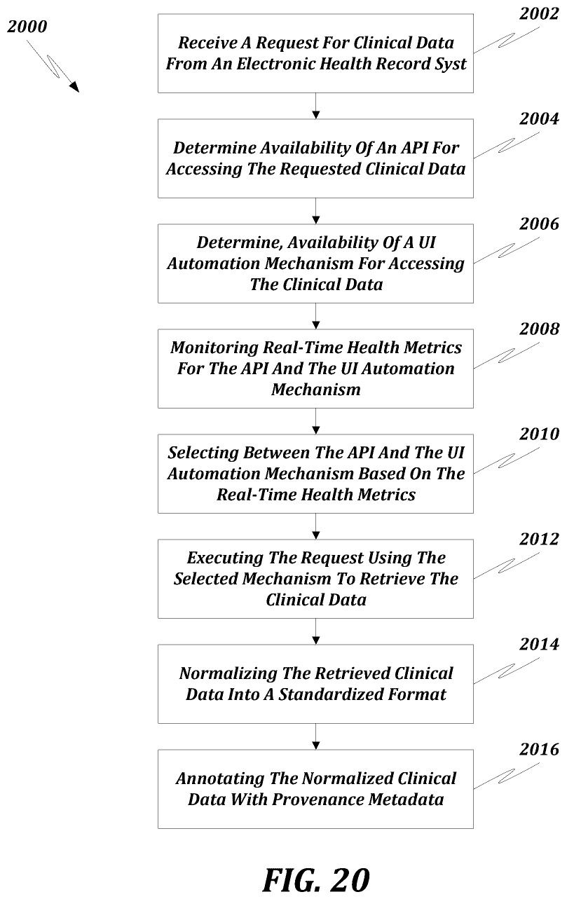

FIG. 20 is a flow chart setting forth the general stages involved in a method 2000 consistent with an embodiment of the disclosure for a method for integrating clinical data across heterogeneous electronic health record systems. Method 2000 may be implemented using a computing device 1500 or any other component associated with platform 100 as described in more detail below with respect to FIG. 15.

Method 2000 may begin at stage 2002 with receiving a request for clinical data from an electronic health record system. In some embodiments, this receiving stage may occur when a healthcare provider initiates a data access operation through the user interface. For example, a physician may request laboratory results for a specific patient by selecting the patient's record and navigating to the laboratory data section. The processor may receive this request along with parameters specifying the patient identifier, the type of clinical data requested, and any filtering criteria such as date ranges or specific test types. In another embodiment, the request may originate from an automated workflow where the system periodically retrieves updated patient information to maintain current data displays.

The method 2000 may proceed to stage 2004, where the system may determine availability of an application programming interface for accessing the requested clinical data. The processor may query a capability registry that maintains information about which data types and operations are accessible via application programming interfaces for each connected electronic health record system. For instance, when requesting medication lists, the processor may determine that the target EHR system provides a FHIR-compliant API endpoint for medication data retrieval. In cases where laboratory results are requested, the processor may identify that the laboratory information system offers an HL7-based API for accessing test results. The determination may also consider whether the current user has appropriate API access credentials and whether the API endpoint is currently operational.

At stage 2006, the availability of a user interface automation mechanism for accessing the clinical data may be assessed. The processor may evaluate whether automated screen interaction capabilities are configured for the target electronic health record system. For example, when requesting patient demographics, the processor may determine that user interface automation scripts are available to navigate the EHR's patient information screens and extract demographic data. In scenarios involving imaging reports, the processor may identify that automation workflows exist to access the radiology information system through simulated user interactions. The determination may include verifying that automation credentials are valid and that the target system's user interface remains compatible with existing automation scripts.

The method 2000 may advance to stage 2008, which may include monitoring real-time health metrics comprising response times and error rates for both the API and user interface automation mechanisms. The processor may continuously track performance indicators for each access pathway. For API pathways, the monitoring may measure the time required to establish connections, transmit requests, and receive responses, while also tracking HTTP error codes and timeout occurrences. For user interface automation pathways, the monitoring may measure script execution times, screen loading delays, and automation failure rates. In one embodiment, the processor may maintain separate metric streams for different data types, such as tracking laboratory API performance independently from medication API performance.

In stage 2010, system may select between the API and user interface automation mechanism based on the real-time health metrics. The processor may apply decision logic that considers multiple performance factors to choose the optimal access pathway. For instance, if the API pathway shows response times under 2 seconds with error rates below 1%, while the automation pathway shows execution times over 10 seconds with failure rates above 5%, the processor may select the API pathway. In another scenario, if the API is experiencing high latency due to system maintenance, the processor may automatically switch to the user interface automation pathway to maintain data access continuity. The selection process may also incorporate institutional policy constraints that mandate specific pathways for certain data types.

The method 2000 may continue to stage 2012, executing the request using the selected mechanism to retrieve the clinical data. When the API pathway is selected, the processor may format the request according to the API specification, transmit the request to the appropriate endpoint, and process the returned data payload. For example, when retrieving patient allergies via a FHIR API, the processor may construct a properly formatted FHIR query, send it to the allergy resource endpoint, and parse the returned FHIR bundle. When the user interface automation pathway is selected, the processor may execute automation scripts that navigate through the EHR's user interface to locate and extract the requested information. For instance, when accessing discharge summaries, the automation may log into the EHR system, navigate to the patient's record, access the documents section, and extract the relevant summary text.

In stage 2014, the system may normalize the retrieved clinical data into a standardized format. The processor may apply transformation rules to convert data from the source system's native format into a unified representation. For laboratory results retrieved via API, the processor may convert proprietary lab codes to LOINC codes and standardize units of measurement. When processing medication data obtained through user interface automation, the processor may map brand names to generic equivalents and normalize dosage formats. The normalization process may also include data validation to ensure completeness and clinical plausibility of the retrieved information.

The method 2000 may conclude in stage 2016 by annotating the normalized clinical data with provenance metadata indicating the selected mechanism and retrieval timestamp. The processor may embed comprehensive tracking information within the normalized data structure. For data retrieved via API, the metadata may include the API endpoint URL, the API version used, authentication method, and response time. For data obtained through user interface automation, the metadata may include the automation script version, screen navigation path, and extraction method used. The timestamp annotation may record both the time when the request was initiated and when the data retrieval was completed, enabling users to assess data freshness and system performance.

### IV. Computing Device Architecture

The platform 100 may be embodied as, for example, but not be limited to, a website, a web application, a desktop application, and a mobile application compatible with a computing device. The computing device may comprise, but not be limited to, a desktop computer, laptop, a tablet, or mobile telecommunications device (105A, 145A). Moreover, the platform 100 may be hosted on a centralized server, such as, for example, a cloud computing service. Although methods 1300, 1400, 1600, 1700, and 1800 have been described to be performed by a computing device 1500, it should be understood that, in some embodiments, different operations may be performed by different networked elements in operative communication with computing device 1500.

Embodiments of the present disclosure may comprise a system having a memory storage and a processing unit. The processing unit coupled to the memory storage, wherein the processing unit is configured to perform the stages of methods 1300, 1400, 1600, 1700, and 1800.

FIG. 15 is a block diagram of a system including computing device 1500. Consistent with an embodiment of the disclosure, the aforementioned memory storage and processing unit may be implemented in a computing device, such as computing device 1500 of FIG. 15. Any suitable combination of hardware, software, or firmware may be used to implement the memory storage and processing unit. For example, the memory storage and processing unit may be implemented with computing device 1500 or any of other computing devices 1518, in combination with computing device 1500. The aforementioned system, device, and processors are examples and other systems, devices, and processors may comprise the aforementioned memory storage and processing unit, consistent with embodiments of the disclosure.

With reference to FIG. 15, a system consistent with an embodiment of the disclosure may include a computing device, such as computing device 1500. In a basic configuration, computing device 1500 may include at least one processing unit 1502 and a system memory 1504. Depending on the configuration and type of computing device, system memory 1504 may comprise, but is not limited to, volatile (e.g., random access memory (RAM)), non-volatile (e.g., read-only memory (ROM)), flash memory, or any combination. System memory 1504 may include operating system 1505, one or more programming modules 1506, and may include a program data 1507. Operating system 1505, for example, may be suitable for controlling computing device 1500's operation. In one embodiment, programming modules 1506 may include a user interface module, favorites module, recommendation module, predictive analytics module, preventive care module, preparedness module, and communication module. Furthermore, embodiments of the disclosure may be practiced in conjunction with a graphics library, other operating systems, or any other application program and is not limited to any particular application or system. This basic configuration is illustrated in FIG. 15 by those components within a dashed line 1508.

Computing device 1500 may have additional features or functionality. For example, computing device 1500 may also include additional data storage devices (removable and/or non-removable) such as, for example, magnetic disks, optical disks, or tape. Such additional storage is illustrated in FIG. 15 by a removable storage 1509 and a non-removable storage 1510. Computer storage media may include volatile and nonvolatile, removable and non-removable media implemented in any method or technology for storage of information, such as computer readable instructions, data structures, program modules, or other data. System memory 1504, removable storage 1509, and non-removable storage 1510 are all computer storage media examples (i.e., memory storage). Computer storage media may include, but is not limited to, RAM, ROM, electrically erasable read-only memory (EEPROM), flash memory or other memory technology, CD-ROM, digital versatile disks (DVD) or other optical storage, magnetic cassettes, magnetic tape, magnetic disk storage or other magnetic storage devices, or any other medium which can be used to store information and which can be accessed by computing device 1500. Any such computer storage media may be part of device 1500. Computing device 1500 may also have input device(s) 1512 such as a keyboard, a mouse, a pen, a sound input device, a touch input device, etc. Output device(s) 1514 such as a display, speakers, a printer, etc. may also be included. The aforementioned devices are examples and others may be used.

Computing device 1500 may also contain a communication connection 1516 that may allow device 1500 to communicate with other computing devices 1518, such as over a network in a distributed computing environment, for example, an intranet or the Internet. Communication connection 1516 is one example of communication media. Communication media may typically be embodied by computer readable instructions, data structures, program modules, or other data in a modulated data signal, such as a carrier wave or other transport mechanism, and includes any information delivery media. The term “modulated data signal” may describe a signal that has one or more characteristics set or changed in such a manner as to encode information in the signal. By way of example, and not limitation, communication media may include wired media such as a wired network or direct-wired connection, and wireless media such as acoustic, radio frequency (RF), infrared, and other wireless media. The term computer readable media as used herein may include both storage media and communication media.

As stated above, a number of program modules and data files may be stored in system memory 1504, including operating system 1505. While executing on processing unit 1502, programming modules 1506 (e.g., a user interface module, favorites module, recommendation module, predictive analytics module, preventive care module, preparedness module, and communication module) may perform processes including, for example, one or more of method 1300's stages as described above. The aforementioned process is an example, and processing unit 1502 may perform other processes. Other programming modules that may be used in accordance with embodiments of the present disclosure may include electronic mail and contacts applications, word processing applications, spreadsheet applications, database applications, slide presentation applications, drawing or computer-aided application programs, etc.

Generally, consistent with embodiments of the disclosure, program modules may include routines, programs, components, data structures, and other types of structures that may perform particular tasks or that may implement particular abstract data types. Moreover, embodiments of the disclosure may be practiced with other computer system configurations, including hand-held devices, multiprocessor systems, microprocessor-based or programmable consumer electronics, minicomputers, mainframe computers, and the like. Embodiments of the disclosure may also be practiced in distributed computing environments where tasks are performed by remote processing devices that are linked through a communications network. In a distributed computing environment, program modules may be located in both local and remote memory storage devices.

Furthermore, embodiments of the disclosure may be practiced in an electrical circuit comprising discrete electronic elements, packaged or integrated electronic chips containing logic gates, a circuit utilizing a microprocessor, or on a single chip containing electronic elements or microprocessors. Embodiments of the disclosure may also be practiced using other technologies capable of performing logical operations such as, for example, AND, OR, and NOT, including but not limited to mechanical, optical, fluidic, and quantum technologies. In addition, embodiments of the disclosure may be practiced within a general purpose computer or in any other circuits or systems.

Embodiments of the disclosure, for example, may be implemented as a computer process (method), a computing system, or as an article of manufacture, such as a computer program product or computer readable media. The computer program product may be a computer storage media readable by a computer system and encoding a computer program of instructions for executing a computer process. The computer program product may also be a propagated signal on a carrier readable by a computing system and encoding a computer program of instructions for executing a computer process. Accordingly, the present disclosure may be embodied in hardware and/or in software (including firmware, resident software, micro-code, etc.). In other words, embodiments of the present disclosure may take the form of a computer program product on a computer-usable or computer-readable storage medium having computer-usable or computer-readable program code embodied in the medium for use by or in connection with an instruction execution system. A computer-usable or computer-readable medium may be any medium that can contain, store, communicate, propagate, or transport the program for use by or in connection with the instruction execution system, apparatus, or device.

The computer-usable or computer-readable medium may be, for example but not limited to, an electronic, magnetic, optical, electromagnetic, infrared, or semiconductor system, apparatus, device, or propagation medium. More specific computer-readable medium examples (a non-exhaustive list), the computer-readable medium may include the following: an electrical connection having one or more wires, a portable computer diskette, a random access memory (RAM), a read-only memory (ROM), an erasable programmable read-only memory (EPROM or Flash memory), an optical fiber, and quantum computing elements. Note that the computer-usable or computer-readable medium could even be paper or another suitable medium upon which the program is printed, as the program can be electronically captured, via, for instance, optical scanning of the paper or other medium, then compiled, interpreted, or otherwise processed in a suitable manner, if necessary, and then stored in a computer memory.

Embodiments of the present disclosure, for example, are described above with reference to block diagrams and/or operational illustrations of methods, systems, and computer program products according to embodiments of the disclosure. The functions/acts noted in the blocks may occur out of the order as shown in any flowchart. For example, two blocks shown in succession may in fact be executed substantially concurrently or the blocks may sometimes be executed in the reverse order, depending upon the functionality/acts involved.

While certain embodiments of the disclosure have been described, other embodiments may exist. Furthermore, although embodiments of the present disclosure have been described as being associated with data stored in memory and other storage mediums, data can also be stored on or read from other types of computer-readable media, such as secondary storage devices, like hard disks, solid state storage (e.g., USB drive), or a CD-ROM, a carrier wave from the Internet, or other forms of RAM or ROM. Further, the disclosed methods'stages may be modified in any manner, including by reordering stages and/or inserting or deleting stages, without departing from the disclosure.

All rights including copyrights in the code included herein are vested in and the property of the Applicant. The Applicant retains and reserves all rights in the code included herein, and grants permission to reproduce the material only in connection with reproduction of the granted patent and for no other purpose.

### Other Embodiments

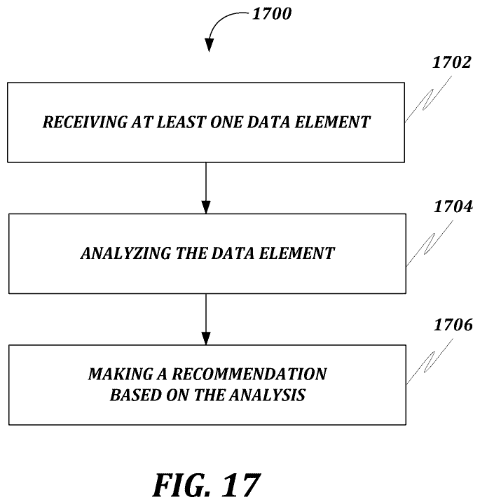

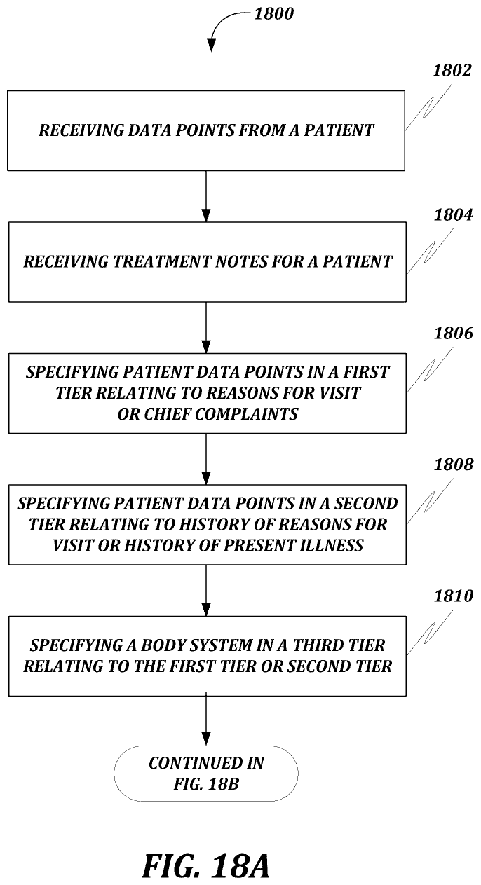

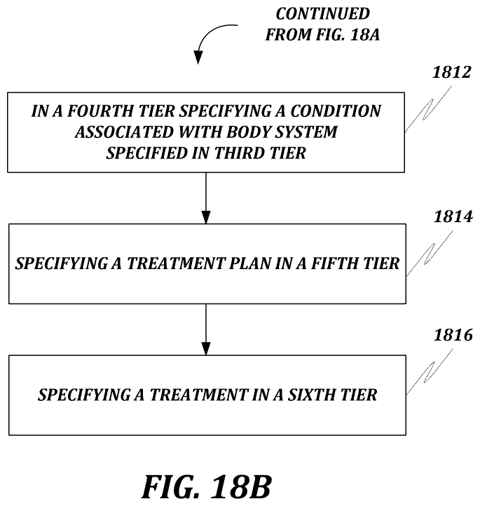

The aforementioned modules may be used in various combinations with one another. The modules can also be used together as separate elements of a system working together but not contained within the same component. In other embodiments, the modules can be contained within the same component or used as an element or elements of a system. In yet other embodiments, the functionality for any of the aforementioned modules can be embodied in one individual module alone or collectively in a portion of the modules. The modules can be embodied as software, hardware or a combination thereof. As described in the present disclosure, the modules can also be collectively embodied in one device, server, computer readable medium, or hardware element. FIGS. 17 and 18 illustrate various additional methods available to be implemented consistent with embodiments of the present disclosure.

### Other Capabilities

The modules may further be configured to utilize existing technologies known to one of ordinary skill in the art. Mobile devices, laptops, cameras, smartphones, computers, sensors, sensory devices, personal sensory devices, microphones, electronic devices, and other devices may be utilized with any of the aforementioned modules. By way of non-limiting example, during a patient encounter, a camera, smartphone, mobile device, computer, or electronic device can be used to capture audio, video, photographs, or other media and/or multimedia content associated with the encounter. In another non-limiting example, alerts generated using the predictive analytics module may provide a patient with a video message from his or her practitioner with delivery of the preventive action. By way of another non-limiting example, alerts generated using the predictive analytics module may provide a practitioner with a video snippet from the news report, a snippet from a news article, a photograph of an affected patient, or other media with delivery of the preemptive preparedness action. This information can be stored for use with the server, external data or other components of the system.

### V. Aspects

The following disclose various Aspects of the present disclosure. The various Aspects are not to be construed as patent claims unless the language of the Aspect appears as a patent claim. The Aspects describe various non-limiting embodiments of the present disclosure.

### Aspects of the User Interface Module

The following disclose various Aspects of the present disclosure. The various Aspects are not to be construed as patent claims unless the language of the Aspect appears as a patent claim. The Aspects describe various non-limiting embodiments of the present disclosure.

Aspect 1. An input system comprising, but not limited to, at least two stages of assisted data entry, the input system comprising: a first stage of input for receiving data points from a patient; a second stage of input for receiving treatment notes for the patient, and wherein the first stage of input comprises a plurality of tiers for specifying the data points for the patient, the plurality of tiers comprising at least one of the following: a first tier for specifying at least one of the following: reasons for visit/chief
complaints; a second tier for specifying at least one of the following: History of Reason for visit/History of Present Illness (HPI); a third tier for specifying a body system associated with at least one of the following: the first tier and the second tier; a fourth tier for specifying a condition associated with the body system specified in the third tier; wherein the second stage of input comprises a plurality of tiers for specifying the treatment notes for the patient, the plurality of tiers comprising at least one of the following: a fifth tier for specifying a treatment plan; and a sixth tier for specifying a treatment in accordance to the treatment plan.

Aspect 2. The input system of Aspect 1 wherein the first stage of input and the second stage of input are compiled into a patient encounter note.

Aspect 3. The input system of Aspect 1 wherein a completion of the patient encounter note is facilitated, at least in part, by at least one of the following: a favorites module; a recommendation module; and an analytics module.

### Aspects of the Favorites Module

The following disclose various Aspects of the present disclosure. The various Aspects are not to be construed as patent claims unless the language of the Aspect appears as a patent claim. The Aspects describe various non-limiting embodiments of the present disclosure.

Aspect 1. An input system comprising, but not limited to, at least two stages of assisted data entry, the input system comprising: providing a first interface for receiving data points associated with a patient encounter, wherein the first interface comprises: an input tier for receiving data points from a user; and providing a second interface for assistant with an entry of the data points into the first interface, wherein the second interface comprises: a plurality of suggested data points made available for user selection, wherein at least one selected suggested data point is configured to populate a currently selected input tier of the first interface.

Aspect 2. The input system of Aspect 1, wherein the first interface further comprises a sub-input tier for categorizing the received data points from the user within the input tier.

Aspect 3. The input system of Aspect 1, wherein the second interface further comprises a plurality of suggested data point categories configured to populate a currently selected input tier of the first interface.

Aspect 4. The input system of Aspect 1, wherein the plurality of suggested data point categories are configured to populate a/the currently selected input tier of the first interface as a sub-input tier.

Aspect 5. The input system of Aspect 4, wherein the sub-input tier is configured to categorize data point received within the input tier of the first interface.

Aspect 6. The input system of Aspect 4, wherein the suggested data points are tailored to a currently selected input tier in the first interface.

Aspect 7. The input system of Aspect 6, wherein the suggested data point categories are tailored to a currently selected input tier in the first interface.

Aspect 8. The input system of Aspect 4, wherein the suggested data points are based on at least one of the following: analysis performed on previous inputs for a current patient encounter; analysis performed on previous patient encounters for the same patient; and analysis performed on previous patient encounters for a plurality of patients.

Aspect 9. The input system of Aspect 8, Wherein the analysis performed on previous inputs for a current patient encounter comprises analyzing inputs made in a current input tier.

Aspect 10. The input system of Aspect 8, wherein the analysis performed on previous inputs for a current patient encounter comprises analyzing completed input tiers for the current patient encounter.

Aspect 11. The input system of Aspect 10, wherein the plurality of patient encounter data used for the analysis is accessed through an internal dataset.

Aspect 12. The input system of Aspect 10, wherein the plurality of patient encounter data used for the analysis is accessed through an external dataset.

Aspect 13. The input system of Aspect 10, wherein the analysis is configured to determine which data points frequently occur together within the input tier.

Aspect 14. The input system of Aspect 10, wherein the analysis is configured to determine a frequency with which the data points were previously used within the input tier.

Aspect 15. The input system of Aspect 10, wherein the analysis is configured to determine which data points frequently occur together across more than one input tier.

Aspect 16. The input system of Aspect 10, wherein the analysis is configured to determine a frequency with which the data points occur together across more than one input tier.

Aspect 17. The input system of Aspect 10, wherein the analysis is configured to determine which data point categories frequently occur together within the input tier.

Aspect 18. The input system of Aspect 10, wherein the analysis is configured to determine a frequency with which the data point categories were previously used within the input tier.

Aspect 19. The input system of Aspect 10, wherein the analysis is configured to determine which data point categories frequently occur together across more than one input tier.

Aspect 20. The input system of Aspect 10, wherein the analysis is configured to determine a frequency with which the data point categories occur together across more than one input tier.

Aspect 21. The input system of Aspect 10, wherein the second interface further comprises a graphical user interface (GUI) element used to indicate a frequency with which each of the suggested data points has previously been used.

Aspect 22. The input system of Aspect 21, wherein the GUI element is configured to accompany a display for each of the suggested data points.

Aspect 23. The input system of Aspect 21, wherein a color of the GUI element is configured to convey an occurrence frequency of the accompanying suggested data point.

Aspect 24. The input system of Aspect 21, wherein an intensity of the color of the GUI element is configured to convey an occurrence frequency of the accompanying suggested data point.

### Aspects of Recommendation Module

The following disclose various Aspects of the present disclosure. The various Aspects are not to be construed as patent claims unless the language of the Aspect appears as a patent claim. The Aspects describe various non-limiting embodiments of the present disclosure.

Aspect 1. A method for provided improved patient diagnostics, the method comprising:
receiving at least one data point in a patient encounter note during a patient diagnostic process; establishing a patient profile based on the at least one data point in the patient encounter note; analyzing the patient profile; recommending, based on the analysis, at least one of the following: a diagnostic question, a recommended treatment, and a recommended action.

Aspect 2. The method of Aspect 1 wherein the patient diagnostic process is configured to guide a user through inputting data associated with a patient encounter, and wherein a recommendation is provided in furtherance of completing the patient diagnostic process.

Aspect 3. The method of Aspect 1 wherein analyzing the patient profile comprises detecting a correlation in data point entries within the patient encounter note.

Aspect 4. The method of Aspect 1 wherein analyzing the patient profile comprises detecting a correlation in data point entries with other data point entries in other patient encounter notes.

Aspect 5. The method of Aspect 1 wherein analyzing further comprises:
determining at least one patient profile similar to the patient profile associated with the patient encounter note; analyzing data points within at least one patient encounter note associated with the at least one similar patient profile; and determining a recommendation based on the analysis of the patient encounter note and the at least one patient encounter note associated with the at least one similar patient profile.

Aspect 6. The method of Aspect 1 wherein analyzing data points within the at least one patient encounter note associated with the at least one similar patient profile comprises: Time Based
Location Based
Gender Based
Age Based
Medical History Based
Family History Based
Social History Based
Surgical History Based
Based on Demographic Data

Aspect 7. The method of Aspect 1 wherein analyzing data points within the at least one patient encounter note associated with the at least one similar patient profile comprises: previous encounters for each patient profile; previous diagnosis for each patient profile; and previous treatments for each patient profile.

Aspect 8. The method of Aspect 1 wherein the suggested data points are based on at least one of the following: analyzing previous inputs for a current patient encounter; and analyzing performed on previous patient encounters for the same patient; and analyzing performed on previous patient encounters for a plurality of patients.

Aspect 9. The method of Aspect 1 wherein analyzing data points within the at least one patient encounter note associated with the at least one similar patient profile comprises: Reasons for visit/chief complaints for each encounter note in each patient profile; and History of Reason for visit/History of Present Illness (HPI) for each encounter note in each patient profile.

Aspect 10. The method of Aspect 1 wherein analyzing the patient profile comprises accessing an external database.

Aspect 11. The method of Aspect 1 wherein accessing the external database comprises employing an application programming interface (API).

Aspect 12. The method of Aspect 1 wherein accessing the external database comprises accessing at least one of the following: Medical Encyclopedias, and WHO databases.

Aspect 13. The method of Aspect 1 wherein analyzing the patient profile comprises accessing environmental sensor data.

Aspect 14. The method of Aspect 12 wherein accessing environmental sensor data comprises accessing data obtained from at least one of the following: weather sensors, air quality sensors, and water sensors.

Aspect 15. The method of Aspect 1 wherein recommending, based on the analysis, comprises at least one of the following: displaying question to ask; receiving validation that question has been asked; providing data point entry for inputting an answer; and recording the data point entry in the encounter note.

Aspect 16. The method of Aspect 15 wherein providing the data point entry comprises updating a display of suggested data point entries in a favorites module.

Aspect 17. The method of Aspect 5 wherein recommending, based on the analysis, comprises providing at least one treatment option for entry into the encounter note.

Aspect 18. The method of Aspect 17 wherein providing the at least one treatment option for entry into the encounter note comprises updating a display of suggested data point entries in a favorites module.

Aspect 19. The method of Aspect 1 wherein recommending, based on the analysis, comprises at least one of the following: displaying a recommended action to execute; receiving validation that action has been executed; providing data point entry for updating the encounter note; and recording the data point entry in the encounter note upon selection of the data point entry.

Aspect 20. The method of Aspect 19 wherein providing the data point entry comprises updating a display of suggested data point entries in a favorites module.

### Aspects of Preventive Care Sub-module

The following disclose various Aspects of the present disclosure. The various Aspects are not to be construed as patent claims unless the language of the Aspect appears as a patent claim. The Aspects describe various non-limiting embodiments of the present disclosure.

Aspect 1. A method for providing preventive actions to patients, the method comprising: receiving at least one data element associated with a plurality of patient data; analyzing the patient data; determining, based on the analysis, whether a set of rules and conditions may cause an adverse effect on a patient; predicting, based on the analysis, at least one of the following: a preventive suggestion, and a preventive action.

Aspect 2. The method of Aspect 1 wherein the data elements further comprise at least one of: coded records, patient encounter data, patient monitoring data, external databases, and external diagnostic data.

Aspect 3. The method of Aspect 1 wherein analyzing data elements is based on the rules and conditions.

Aspect 4. The method of Aspect 1 wherein analyzing further comprises: determining whether a data element meets a predetermined threshold.

Aspect 5. The method of Aspect 4 wherein the predetermined threshold is associated with the set of conditions.

Aspect 6. The method of Aspect 3 wherein determining further comprises: determining whether there is a high probability that a known set of conditions will adversely impact one or more patients.

Aspect 7. The method of Aspect 6 wherein analyzing further comprises: detecting a correlation between an external patient data element meeting the predetermined threshold and a patient data element.

Aspect 8. The method of Aspect 6 wherein determining further comprises: detecting a correlation between an external patient data element meeting the predetermined threshold and a patient data element.

Aspect 9. The method of Aspect 6 wherein determining further comprises: a positive correlation between an external patient data element meeting the predetermined threshold and a patient data element triggers an alert.

Aspect 10. The method of Aspect 7 wherein analyzing further comprises determining whether patients should receive an alert based on patient profile data.

Aspect 11. The method of Aspect 1 wherein analyzing the patient profile comprises detecting a correlation in data point entries with other data point entries in other patient encounter notes.

Aspect 12. The method of Aspect 1 wherein analyzing further comprises: determining at least one patient profile similar to the patient profile associated with the set of conditions; analyzing data points within at least one patient data element associated with the at least one determined set of conditions; and determining a preventive action based on the analysis of the patient data element and the at least one external data element associated with the at least one condition.

### Aspects of the Preparedness Sub-module

The following disclose various Aspects of the present disclosure. The various Aspects are not to be construed as patent claims unless the language of the Aspect appears as a patent claim. The Aspects describe various non-limiting embodiments of the present disclosure.

Aspect 1. A method for providing preemptive preparedness actions to practitioners, the method comprising: receiving at least one data element associated with a plurality of patient data; analyzing the data element; determining, based on the analysis, whether an occurrence of an event may cause an adverse effect on a patient; predicting, based on the analysis, at least one of the following: a preemptive preparedness suggestion, and a preemptive preparedness action; providing the practitioner with at least one of the following: the preemptive preparedness suggestion, and the preemptive preparedness action.

Aspect 2. A method for providing preemptive preparedness actions to practitioners, the method comprising: receiving at least one data element associated with at least one of: a plurality of patient data, a plurality of external data, a plurality of patient profile data, a plurality of encounter notes, patient tracking data, other data; analyzing the data element; determining, based on the analysis, whether an occurrence of an event may cause an adverse effect on a patient; predicting, based on the analysis, at least one of the following: a preemptive preparedness suggestion, and a preemptive preparedness action; providing the practitioner with at least one of the following: a preemptive preparedness suggestion, and a preemptive preparedness action.

Aspect 3. The method of Aspect 1 or Aspect 2 further comprising: wherein determining further comprises whether a probability of the occurrence of an event has met or exceeded a predetermined threshold.

Aspect 4. The method of Aspect 3 wherein the predetermined threshold is associated with the set of conditions.

Aspect 5. The method of Aspect 3 wherein determining further comprises: determining whether there is a high probability that a known set of conditions will adversely impact one or more patients.

Aspect 6. The method of Aspect 3 wherein analyzing further comprises: detecting a correlation between an external patient data element meeting the predetermined threshold, a patient data element, and data elements associated with a practitioner.

Aspect 7. The method of Aspect 3 wherein determining further comprises: detecting a correlation between impacted patients and data elements associated with a practitioner.

Aspect 8. The method of Aspect 3 wherein determining further comprises: a positive correlation between an external patient data element meeting the predetermined threshold and a patient data element triggers a practitioner alert.

Aspect 9. The method of Aspect 3 wherein analyzing further comprises: determining whether practitioners should receive an alert based on patient profile data.

Aspect 10. The method of Aspect 3 wherein analyzing the patient profile comprises: detecting a correlation in data point entries with other data point entries in other patient encounter notes.

Aspect 11. The method of Aspect 3 wherein analyzing further comprises: determining at least one patient profile similar to the patient profile associated with the set of conditions; analyzing data points within at least one patient data element associated with the at least one determined set of conditions; and determining a preemptive preparedness action based on the analysis of the patient data element and the at least one external data element associated with the at least one condition.

Aspect 12. The method of Aspect 3 wherein analyzing further comprises: determining at least one patient profile similar to the patient profile associated with the set of conditions; analyzing data points within at least one patient data element associated with the at least one determined set of conditions; and determining a preemptive preparedness action based on the analysis of the patient data element and the at least one external data element associated with the at least one condition.

Aspect 13. The method of Aspect 3 wherein data elements associated with a practitioner further comprises at least one of: practitioner schedule, practitioner availability, practitioner support staff schedule, practitioner support staff availability, available/stocked supplies, needed supplies, ordered supplies, supplies scheduled for delivery, beds available, beds needed, quarantine rooms available, quarantine rooms needed, or other data element associated with the practitioner.

Aspect 14. The method of Aspect 3 wherein data elements associated with impacted patients further comprises at least one of: potentially mild symptoms, potentially moderate symptoms, potentially severe symptoms, or other data element associated with impacted patients.

Aspect 15. The method of Aspect 3 wherein the analysis, based on the correlation of data, further comprises determining at least one of: a preemptive preparedness action, a preemptive preparedness suggestion, a series of preemptive preparedness actions, a series of preemptive preparedness suggestions.

Aspect 16. The method of Aspect 3 wherein the preemptive preparedness action further comprises alerting the practitioner to prepare for at least one of: a high number of walk-ins, a moderate number of walk-ins, a low number of walk-ins, an average number of walk-ins, or other preemptive preparedness action.

Aspect 17. The method of Aspect 3 wherein the preemptive preparedness action further comprises informing the practitioner of the occurrence of at least one of: an outbreak, an epidemic, an extreme crisis, a natural disaster, or other emergent event.

Aspect 18. The method of Aspect 3 wherein the preemptive preparedness action further comprises advising the practitioner to prepare for at least one of the following: a high volume of patient calls, a moderate volume of patient calls, a low volume of patient calls, an average number of patient calls, or other preemptive preparedness action.

Aspect 19. The method of Aspect 3 wherein the preemptive preparedness action further comprises suggesting to the practitioner at least one of the following: schedule more practitioner support staff, schedule more on duty practitioners, maintain level of practitioner support staff, reduce level of practitioner support staff, maintain level of available practitioners, reduce level of available practitioners, order more supplies, stock delivered supplies, increase number of beds available, maintain average number of beds available, increase number of quarantine rooms available, maintain number of quarantine rooms available, or other preemptive preparedness action.

Aspect 20. A method for providing preemptive preparedness actions to practitioners, the method comprising: receiving at least one data element associated with at least one of: a plurality of patient data, a plurality of external data, a plurality of patient profile data, a plurality of encounter notes, a plurality of patient tracking data, other data; analyzing the data element, determining whether a practitioner qualifies to receive an alert associated with patient, providing an alert to the practitioner.

Aspect 21. The method of Aspect 20 wherein determining further comprises determining whether the practitioner is at least one or more of: a general practitioner, a specialized practitioner.

Aspect 22. A method for providing preemptive preparedness actions to practitioners, the method comprising: receiving at least one data element associated with at least one of: a plurality of patient data, a plurality of external data, a plurality of patient profile data, a plurality of encounter notes, a plurality of patient tracking data, other data; analyzing patient data for a plurality of patients; determining whether one or more practitioners qualifies to receive an alert; providing an alert to the one or more practitioners.

Aspect 23. The method of Aspect 22 wherein analyzing an external data element further comprises determining whether the probability of the occurrence an event has reached a predetermined threshold.

Aspect 24. The method of Aspect 23 wherein the occurrence of an event impacts patients or a subset of patients.

Aspect 25. The method of Aspect 23 wherein the occurrence of an event is at least one of: high pollen count, poor air quality, high smog level, high lead level in public water supply, poisonous gas released, high radiation levels, terror attack, fire, flooding, earthquake, tornado, heat wave, drought, or other emergent event.

Aspect 26. A computer readable medium comprising, but not limited to, at least one of the following: an input methodology comprising, but not limited to, at least two stages of assisted data entry; and an input assistance methodology comprising, but not limited to, at least one of the following: a favorites module, a recommendation module, an analytics module; and a server in communication with the input methodology and the input assistance methodology.

Aspect 27. The computer readable medium of Aspect 26 further comprising: an improved patient diagnostics method comprising: receiving at least one data element associated with at least one of the following: a plurality of patient data, and at least one data point in a patient encounter note during a patient diagnostic process; and analyzing the data element.

Aspect 28. The computer readable medium of Aspect 26, the input assistance methodology further comprising: recommending, based on an analysis, at least one of the following: a diagnostic question, a recommended treatment, and a recommended action.

Aspect 29. The computer readable medium of Aspect 26 further comprising: an analytics method comprising: determining, based on an analysis, whether an occurrence of an event may cause an adverse effect on a patient; predicting, based on an analysis, at least one of the following: a preventive suggestion, a preventive action, a preemptive preparedness suggestion, and a preemptive preparedness action.

Aspect 30. The computer readable medium of Aspect 29 further comprising: an action generating method comprising: providing a patient or a practitioner with at least one of the following: the preventive suggestion, the preventive action, the preemptive preparedness suggestion, and the preemptive preparedness action.

Aspect 31. An input system comprising, but not limited to, at least two stages of assisted data entry, the input system comprising: a first stage of input for receiving data points from a patient; a second stage of input for receiving treatment notes for the patient, and wherein the first stage of input comprises a plurality of tiers for specifying the data points for the patient, the plurality of tiers comprising at least one of the following: a first tier for specifying at least one of the following: reasons for visit, chief complaints; a second tier for specifying at least one of the following: History of Reason for visit, History of Present Illness (HPI); a third tier for specifying a body system associated with at least one of the following: the first tier, and the second tier; a fourth tier for specifying a condition associated with the body system specified in the third tier; wherein the second stage of input comprises a plurality of tiers for specifying the treatment notes for the patient, the plurality of tiers comprising at least one of the following: a fifth tier for specifying a treatment plan; and a sixth tier for specifying a treatment in accordance to the treatment plan.

Aspect 32. The input system of Aspect 31, further comprising: wherein the first stage of input and the second stage of input are compiled into a patient encounter note.

Aspect 33. The input system of Aspect 31, further comprising: wherein a completion of the patient encounter note is facilitated, at least in part, by at least one of the following: a favorites module; a recommendation module; and an analytics module.

Aspect 34. The input system of Aspect 31, wherein the input system further comprises: providing a first interface for receiving data points associated with a patient encounter, wherein the first interface comprises: an input tier for receiving data points from a user; and providing a second interface for assistant with an entry of the data points into the first interface, wherein the second interface comprises: a plurality of suggested data points made available for user selection, wherein at least one selected suggested data point is configured to populate a currently selected input tier of the first interface.

Aspect 35. The input system of Aspect 34, wherein the suggested data points are based on at least one of the following: analyzing previous inputs for a current patient encounter; and analyzing performed on previous patient encounters for the same patient; and analyzing performed on previous patient encounters for a plurality of patients.

Aspect 36. The input system of Aspect 31, wherein an analysis is configured to determine at least one of the following: which data points frequently occur together within at least one of: the first tier, the second tier, the third tier, the fourth tier, the fifth tier, the sixth tier; and a frequency with which the data points were previously used within at least one of: the first tier, the second tier, the third tier, the fourth tier, the fifth tier, the sixth tier; and which data points frequently occur together across more than at least one of: the first tier, the second tier, the third tier, the fourth tier, the fifth tier, the sixth tier; and which the data points occur together across more than within at least one of: the first tier, the second tier, the third tier, the fourth tier, the fifth tier, the sixth tier.

Aspect 37. A method comprising: an input method comprising: providing a first interface for receiving data points associated with a patient encounter, wherein the first interface comprises: an input tier for receiving the data points from a user; and providing a second interface for assisting with an entry of the data points into the first interface, wherein the second interface comprises: a plurality of suggested data points made available for user selection, wherein at least one selected suggested data point is configured to populate a currently selected input tier of the first interface; and an improved patient diagnostics method comprising: receiving at least one data element associated with at least one of the following: a plurality of patient data, and at least one data point in a patient encounter note during a patient diagnostic process; analyzing the data element; an input assistance method comprising: recommending, based on the analysis, at least one of the following: a diagnostic question, a recommended treatment, and a recommended action an analytics method comprising: determining, based on an analysis, whether an occurrence of an event may cause an adverse effect on a patient; predicting, based on the analysis, at least one of the following: a preventive suggestion, a preventive action, a preemptive preparedness suggestion, and a preemptive preparedness action; an action generating method comprising: providing the patient or practitioner with at least one of the following: the preventive suggestion, the preventive action, the preemptive preparedness suggestion, and the preemptive preparedness action.

Aspect 38. The method of Aspect 37, further comprising: wherein the patient diagnostic process is configured to guide a user through inputting data associated with a patient encounter, and wherein a recommendation is provided in furtherance of completing the patient diagnostic process.

Aspect 39. The method of Aspect 37 wherein analyzing further comprises: determining at least one patient profile similar to a patient profile associated with the patient encounter note; analyzing data points within at least one patient encounter note associated with the at least one similar patient profile; and determining a recommendation based on the analysis of the patient encounter note and the at least one patient encounter note associated with the at least one similar patient profile.

Aspect 40. The method of Aspect 37 further comprising: wherein analyzing data points within the at least one patient encounter note associated with the at least one similar patient profile comprises analyzing at least one of the following: previous encounters for a patient profile; previous diagnosis for the patient profile; and previous treatments for the patient profile.

Aspect 41. The method of Aspect 37 further comprising: receiving at least one data element associated with at least one of: a plurality of patient data, a plurality of external data, a plurality of patient profile data, a plurality of encounter notes, a plurality of patient tracking data, other data; analyzing patient data for a plurality of patients; and determining whether one or more practitioners qualifies to receive an alert; and providing the alert to the one or more practitioners.

Aspect 42. The method of Aspect 37 further comprising: determining whether one or more patients qualifies to receive an alert; and providing the alert to the one or more patients.

Aspect 43. The method of Aspect 37, wherein analyzing further comprises: determining at least one patient profile similar to a patient profile associated with a set of conditions; analyzing data points within at least one patient data element associated with at least one determined set of conditions; and determining a preemptive preparedness action based on the analysis of the at least one patient data element and the at least one external data element associated with at least one condition.

Aspect 44. The method of Aspect 43, further comprising: wherein determining further comprises determining whether there is a high probability that a known set of conditions will adversely impact one or more patients; wherein analyzing further comprises determining whether a probability of an occurrence an event has reached a predetermined threshold; and wherein determining further comprises a positive correlation between an external patient data element meeting the predetermined threshold and a patient data element triggers an alert.

Aspect 45. The method of Aspect 44, further comprising: wherein the occurrence of an event is at least one of: high pollen count, poor air quality, high smog level, high lead level in public water supply, poisonous gas released, high radiation levels, terror attack, fire, flooding, earthquake, tornado, heat wave, drought, or other emergent event.

While the specification includes examples, the disclosure's scope is indicated by the following claims. Furthermore, while the specification has been described in language specific to structural features and/or methodological acts, the claims are not limited to the features or acts described above. Rather, the specific features and acts described above are disclosed as example for embodiments of the disclosure.

Insofar as the description above and the accompanying drawing disclose any additional subject matter that is not within the scope of the claims below, the disclosures are not dedicated to the public and the right to file one or more applications to claims such additional disclosures is reserved.

The following is claimed:

1. A clinical data integration system comprising:
a processor;
a memory coupled to the processor;
a routing engine executed by the processor and configured to:
receive a data access request for clinical information from a first electronic health record (EHR) system,
determine availability of an application programming interface (API) for the data access request,
determine availability of a user interface automation pathway for the data access request,
select between the API and the user interface automation pathway based on real-time health metrics of each pathway, and
execute the data access request using the selected pathway;
a health monitoring module executed by the processor and configured to:
continuously monitor response times and error rates for the API and the user interface automation pathway, and
generate the real-time health metrics based on the monitored response times and error rates; and
a data normalization module executed by the processor and configured to:
receive clinical data from the executed data access request,
normalize the clinical data into a standardized format, and
annotate the normalized clinical data with provenance information indicating the selected pathway and a timestamp of retrieval.

2. The system of claim 1, wherein the routing engine is further configured to automatically switch from the API to the user interface automation pathway when the real-time health metrics indicate the API response time exceeds a predetermined threshold.

3. The system of claim 1, wherein the routing engine is further configured to automatically switch from the user interface automation pathway to a cached data source when the real-time health metrics indicate the user interface automation pathway is unavailable.

4. The system of claim 1, further comprising:
an intent processing module executed by the processor and configured to:
receive a natural language input from a user,
map the natural language input to a predefined intent from a whitelist of approved intents,
reject natural language inputs that do not correspond to any predefined intent in the whitelist, and
convert the predefined intent into the data access request.

5. The system of claim 4, wherein the intent processing module is configured to decompose complex predefined intents into a sequence of primitive operations, each primitive operation being executable via the API or the user interface automation pathway.

6. The system of claim 1, further comprising:
a provider profile module executed by the processor and configured to:
maintain a provider-specific interaction profile associated with a provider identity,
map the provider identity to local identities across multiple EHR systems, and
apply the provider-specific interaction profile consistently across the multiple EHR systems regardless of which EHR system is currently active.

7. The system of claim 6, wherein the provider-specific interaction profile comprises layout preferences, workflow configurations, and presentation defaults that persist across different EHR systems.

8. A method for integrating clinical data across heterogeneous electronic health record systems, the method comprising:
receiving, by a processor, a request for clinical data from a first electronic health record (EHR) system;
determining, by the processor, availability of an application programming interface (API) for accessing the clinical data;
determining, by the processor, availability of a user interface automation mechanism for accessing the clinical data;
monitoring, by the processor, real-time health metrics comprising response times and error rates for the API and the user interface automation mechanism;
selecting, by the processor, between the API and the user interface automation mechanism based on the real-time health metrics;
executing, by the processor, the request using the selected mechanism to retrieve the clinical data;
normalizing, by the processor, the retrieved clinical data into a standardized format; and
annotating, by the processor, the normalized clinical data with provenance metadata indicating the selected mechanism and retrieval timestamp.

9. The method of claim 8, further comprising:
automatically switching from the API to the user interface automation mechanism when the real-time health metrics indicate the API is experiencing degraded performance.

10. The method of claim 8, further comprising:
automatically switching to a cached data source when both the API and the user interface automation mechanism are unavailable, wherein the cached data source is labeled with freshness indicators.

11. The method of claim 8, further comprising:
receiving a natural language input from a user;
constraining the natural language input to a predefined set of whitelisted intents;
rejecting natural language inputs that fall outside the predefined set of whitelisted intents; and
converting an accepted whitelisted intent into the request for clinical data.

12. The method of claim 11, wherein constraining the natural language input comprises mapping the natural language input deterministically to exactly one whitelisted intent to prevent unpredictable system behavior.

13. The method of claim 8, further comprising:
maintaining a provider-specific interaction profile linked to a provider identity;
mapping the provider identity to local user accounts across multiple different EHR systems; and
applying the provider-specific interaction profile consistently when the provider accesses any of the multiple different EHR systems.

14. The method of claim 13, wherein the provider-specific interaction profile comprises persistent layout preferences and workflow configurations that remain consistent across different EHR vendor platforms.

15. A non-transitory computer-readable storage medium storing instructions that, when executed by a processor, cause the processor to:
receive a clinical data access request targeting a first electronic health record (EHR) system;
evaluate availability of multiple data access pathways comprising an application programming interface (API) pathway and a user interface automation pathway;
monitor real-time performance metrics for each of the multiple data access pathways;
dynamically select one of the multiple data access pathways based on the real-time performance metrics and predefined policy constraints;
execute the clinical data access request using the selected pathway;
normalize retrieved clinical data into a standardized representation; and
tag the normalized clinical data with provenance information identifying the selected pathway and data freshness indicators.

16. The non-transitory computer-readable storage medium of claim 15, wherein the instructions further cause the processor to:
capture a context snapshot comprising current user interface state, active patient context, and applied filters; and
serialize the context snapshot for later restoration to recreate the captured user interface state.

17. The non-transitory computer-readable storage medium of claim 16, wherein the instructions further cause the processor to:
restore the context snapshot by rehydrating the serialized user interface state, active patient context, and applied filters to recreate a previous workspace configuration.

18. The non-transitory computer-readable storage medium of claim 15, wherein the instructions further cause the processor to:
enforce institutional policy constraints that define which clinical data operations are permitted via the API pathway versus the user interface automation pathway based on user role and data sensitivity.

19. The non-transitory computer-readable storage medium of claim 15, wherein the instructions further cause the processor to:
maintain separate health status indicators for different types of clinical data access operations; and
route each clinical data access request based on health status indicators specific to the type of operation being requested.

20. The non-transitory computer-readable storage medium of claim 15, wherein the instructions further cause the processor to:
generate structured clinical documentation by aggregating clinical data from multiple sources via the selected pathway;
embed provenance metadata within the structured clinical documentation indicating data sources and retrieval methods; and
write the structured clinical documentation back to the first EHR system while preserving the embedded provenance metadata.
